(19) United States
(12) Patent Application Publication
(10) Pub. No.: US 2025/0197917 A1
(43) Pub. Date: Jun. 19, 2025

(54) METHOD OF PROTEASE DETECTION

(71) Applicant: GLYMPSE BIO, INC., Cambridge, MA (US)

(72) Inventors: Faycal TOUTI, Cambridge, MA (US); Mehar CHEEMA, Cambridge, MA (US)

(21) Appl. No.: 18/845,308

(22) PCT Filed: Mar. 8, 2023
(86) PCT No.: PCT/US2023/014851

§ 371 Date: Sep. 9, 2024

Related U.S. Application Data
(60) Provisional application No. 63/318,220, filed on Mar. 9, 2022.

(51) Int. Cl. C12Q 1/37

(52) U.S. Cl. CPC C12Q 1/37; G01N 2333/165; G01N 2333/9513; G01N 2469/10; G01N 2800/26

(57) ABSTRACT

The present application provides a synthetic molecule and a method for producing the same, and also provides methods for determining a disease or condition in a subject. The method comprises introducing a synthetic molecule comprising a synthetic polymer and a linker, wherein the linker comprises an organic molecule, a spacer sequence, and a reporter, wherein the reporter is cleaved by an agent present in the environment. Diseases and conditions that can be determined by the method are also described in the present application.

### CROSS REFERENCE

This application claims the benefit of U.S. Provisional Application No. 63/318,220, filed Mar. 9, 2022, which is incorporated herein by reference in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### SUMMARY

Provided herein is a synthetic molecule comprising: a synthetic polymer comprising a core, a plurality of branch points, and a plurality of endpoints; a plurality of linkers, wherein a linker of the plurality of linkers comprises (i) a linker sequence, (ii) a first end, and (iii) a second end, wherein the first end is coupled to an endpoint of the plurality of endpoints; and a plurality of peptide sequences, wherein a peptide sequence of the plurality of peptide sequences is coupled to the second end of the linker, wherein the synthetic molecule is configured to react with an enzyme present in a sample obtained from a subject.

In some cases, the second end comprises a reactive handle. In some cases, each of the plurality of peptide sequences comprise a sequence that is at least 60% homologous to other sequences in the plurality of peptide sequences. In some cases, the plurality of peptide sequences comprises different peptide sequences. In some cases, the plurality of peptide sequences comprises a combination of a first set of peptide sequences and a second set of peptide sequences, wherein each peptide sequence of the first set of peptide sequences is similar, and the second set of peptide sequences comprises different peptide sequences. In some cases, each of the plurality of linkers comprises a spacer coupled to an organic molecule. In some cases, the spacer comprises a PEG sequence. In some cases, the organic molecule comprises an imide, a tetrazine, a cyclooctyne, an azide, an alkyne, a phosphine, a norbornene, a thiol, an alkene, an aldehyde, a hydroxylamine, a diene, a dienophile, a hydroxysuccinimide, or an amine. In some cases, imide comprises formula (I):

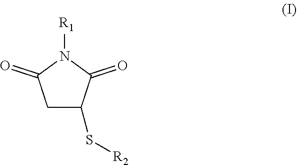

In some cases, the core comprises an amine core. In some cases, the amine core comprises an ethylenediamine core or a polyamidoamine core. In some cases, each of the plurality of branch points, the plurality of endpoints, or a combination thereof, are configured to exhibit different chemical properties from one another. In some cases, the reaction with the enzyme indicates an enzyme activity. In some cases, the enzyme activity comprises a disease-related enzyme activity, a baseline enzyme activity, or a combination thereof. In some cases, the enzyme comprises a protease, and wherein the enzyme activity comprises a protease activity. In some cases, the protease activity indicates a presence of a pathogen, and wherein the presence of the pathogen is associated with a disease. In some cases, the synthetic molecule further comprises a probe. In some cases, the probe is selected from Table 1. In some cases, the synthetic molecule comprises an IEPD dendrimer, and wherein the probe comprises a probe 9 molecule, a probe 102 molecule, a probe 379 molecule, or a combination thereof. In some cases, the synthetic molecule further comprises a plurality of probes. In some cases, the plurality of probes are selected from Table 1. In some cases, the IEPD dendrimer comprises a G4-IEPD dendrimer, a G5-IEPD dendrimer, a G6-IEPD dendrimer, a G7-IEPD dendrimer, a G8-IEPD dendrimer, a G9-IPED dendrimer, or a G10-IEPD dendrimer.

In some cases, the protease is selected from the group consisting of an A20 (TNFa-induced protein 3), an abhydrolase domain containing 4, an abhydrolase domain containing 12, an abhydrolase domain containing 12B, an abhydrolase domain containing 13, an acrosin, an acylaminoacyl-peptidase, a disintegrin and metalloproteinase (ADAM), an ADAM1a, an ADAM2 (Fertilin-b), an ADAM3B, an ADAM4, an ADAM4B, an ADAM5, an ADAM6, an ADAM7, an ADAM8, an ADAM9, an ADAM10, an ADAM11, an ADAM12 metalloprotease, an ADAM15, an ADAM17, an ADAM18, an ADAM19, an ADAM20, an ADAM21, an ADAM22, an ADAM23, an ADAM28, an ADAM29, an ADAM30, an ADAM32, an ADAM33, a disintegrin and metalloproteinase with thrombospondin motifs (ADAMTS), an ADAMTS1, an ADAMTS2, an ADAMTS3, an ADAMTS4, an ADAMTS5/11, an ADAMTS6, an ADAMTS7, an ADAMTS8, an ADAMTS9, an ADAMTS10, an ADAMTS12, an ADAMTS13, an ADAMTS14, an ADAMTS15, an ADAMTS16, an ADAMTS17, an ADAMTS18, an ADAMTS19, an ADAMTS20, an adipocyte-enhancer binding protein 1, an Afg3-like protein 1, an Afg3-like protein 2, an airway-trypsin-like protease, an aminoacylase, an aminopeptidase A, an aminopeptidase B, an aminopeptidase B-like 1, an aminopeptidase MAMS/L-RAP, an aminopeptidase N, an aminopeptidase O, an aminopeptidase P homologue, an aminopeptidase P1, an aminopeptidase PILS, an aminopeptidase Q, an aminopeptidase-like 1, an AMSH/STAMBP, an AMSH-LP/STAMBPL1, an angiotensin-converting enzyme 1 (ACE1), an angiotensin-converting enzyme 2 (ACE2), an angiotensin-converting enzyme 3 (ACE3), an anionic trypsin (II), an apolipoprotein (a), an archaemetzincin-1, an archaemetzincin-2, an aspartoacylase, an aspartoacylase-3, an aspartyl aminopeptidase, an ataxin-3, an ataxin-3 like, an ATP/GTP binding protein 1, an ATP/GTP binding protein-like 2, an ATP/GTP binding protein-like 3, an ATP/GTP binding protein-like 4, an ATP/GTP binding protein-like 5, an ATP23 peptidase, an autophagin-1, an autophagin-2, an autophagin-3, an autophagin-4, an azurocidin, a beta lactamase, a beta-secretase 1, a beta-secretase 2, a bleomycin hydrolase, a brain serine proteinase 2, a BRCC36 (BRCA2-containing complex, sub 3), a calpain, a calpain 1, a calpain 2, a calpain 3, a calpain 4, a calpain 5, a calpain 6, a calpain 7, a calpain 7-like, a calpain 8, a calpain 9, a calpain 10, a calpain 11, a calpain 12, a calpain 13, a calpain 14, a calpain 15 (Solh protein), a cysteine protease, a carboxypeptidase A1, a carboxypeptidase A2, a carboxypeptidase A3, a carboxypeptidase A4, a carboxypeptidase A5, a carboxypeptidase A6, a carboxypeptidase B, a carboxypeptidase D, a carboxypeptidase E, a carboxypeptidase M, a carboxypeptidase N, a carboxypeptidase O, a carboxypeptidase U, a carboxypeptidase X1, a carboxypeptidase X2, a carboxypeptidase Z, a carnosine dipeptidase 1, a carnosine dipeptidase 2, a caspase recruitment domain family, member 8, a caspase, a caspase-1, a caspase-2, a caspase-3, a caspase-4/11, a caspase-5, a caspase-6, a caspase-7, a caspase-8, a caspase-9, a caspase-10, a caspase-12, a caspase-14, a caspase-14-like, a casper/FLIP, a cathepsin, a cathepsin A (CTSA), a cathepsin B (CTSB), a cathepsin C (CTSC), a cathepsin D (CTSD), a cathepsin E (CTSE), a cathepsin F, a cathepsin G, a cathepsin H (CTSH), a cathepsin K (CTSK), a cathepsin L (CTSL), a cathepsin L2, a cathepsin O, a cathepsin S (CTSS), a cathepsin V (CTSV), a cathepsin W, a cathepsin Z (CTSZ), a cationic trypsin, a cezanne/OTU domain containing 7B, a cezanne-2, a CGI-58, a chymase, a chymopasin, a chymosin, a chymotrypsin B, a chymotrypsin C, a coagulation factor IXa, a coagulation factor VIIa, a coagulation factor Xa, a coagulation factor XIa, a coagulation factor XIIa, a collagenase 1, a collagenase 2, a collagenase 3, a complement protease C1r serine protease, a complement protease C1s serine protease, a complement C1r-homolog, a complement component 2, a complement component C1ra, a complement component C1sa, a complement factor B, a complement factor D, a complement factor D-like, a complement factor I, a COPS6, a corin, a CSN5 (JAB1), a cylindromatosis protein, a cytosol alanyl aminopep.-like 1, a cytosol alanyl aminopeptidase, a DDI-related protease, a DECYSIN, a Der1-like domain family, member 1, a Der1-like domain family, member 2, a Der1-like domain family, member 3, a DESC1 protease, a desert hedgehog protein, a desumoylating isopeptidase 1, a desumoylating isopeptidase 2, a dihydroorotase, a dihydropyrimidinase, a dihydropyrimidinase-related protein 1, a dihydropyrimidinase-related protein 2, a dihydropyrimidinase-related protein 3, a dihydropyrimidinase-related protein 4, a dihydropyrimidinase-related protein 5, a DINE peptidase, a dipeptidyl peptidase (DPP), a dipeptidyl peptidase (DPP1), a dipeptidyl-peptidase 4 (DPP4), a dipeptidyl-peptidase 6 (DPP6), a dipeptidyl-peptidase 8 (DPP8), a dipeptidyl-peptidase 9 (DPP9), a dipeptidyl-peptidase II, a dipeptidyl-peptidase III, a dipeptidyl-peptidase 10 (DPP10), a DJ-1, a DNA-damage inducible protein, a DNA-damage inducible protein 2, a DUB-1, a DUB-2, a DUB2a, a DUB2a-like, a DUB2a-like2, a DUB6, or a combination thereof.

In some cases, the protease is selected from the group consisting of a T cell protease, a complement protease, a fibrosis protease, and an inflammation-related protease. In some cases, the synthetic molecule further comprises a carrier. In some cases, the carrier comprises a native, labeled or synthetic protein, a synthetic chemical polymer of precisely known chemical composition or with a distribution around a mean molecular weight, an oligonucleotide, a phosphorodiamidate morpholino oligomer (PMO), a foldamer, a lipid, a lipid micelle, a nanoparticle, a solid support made of polystyrene, polypropylene or any other type of plastic, or any combination thereof. In some cases, the linker comprises a peptide, a carbohydrate, a nucleic acid, a lipid, an ester, a glycoside, a phospholipid, a phosphodiester, a nucleophile/base sensitive linker, a reduction sensitive linker, an electrophile/acid sensitive linker, a metal cleavable linker, an oxidation sensitive linker or a combination thereof. In some cases, the enzyme present in the sample is configured to bind to a binding site on the synthetic molecule, and wherein the synthetic molecule is present in the sample at a concentration of 0.01 nM-0.1M. In some cases, the enzyme is present in the sample at a concentration of between approximately 0.01 nM-1.0 nM. In some cases, the plurality of peptide sequences is configured to have an increased affinity to the enzyme in comparison to a linear peptide sequence not linked to the synthetic molecule. In some cases, the plurality of peptide sequences comprises approximately 1-50 peptides, 50-100 peptides, or 100-150 peptides. In some cases, the plurality of peptide sequences are different, similar, or a combination thereof.

In some cases, the second end comprises a tunable sequence. In some cases, the synthetic molecule is configured react with a paper strip application. In some cases, the synthetic polymer comprises a dendrimer, a multivalent synthetic macromolecule, a nanoparticle scaffold, a polymeric scaffold, a foldamer, a branched peptide, or a synthetic composite nanoparticle. In some cases, the peptide sequence comprises a reporter, a peptide sequence spacer, a reactive handle, and a binding site for the enzyme. In some cases, the reporter comprises a fluorescent molecule. In some cases, the fluorescent molecule comprises a FRET peptide.

In another aspect, provided herein is a method comprising: contacting a body fluid sample obtained from a subject with a synthetic molecule, wherein the synthetic molecule comprises: a dendrimer, a plurality of linkers coupled to the dendrimer, and at least one peptide sequence coupled to the plurality of linkers, wherein the at least one peptide sequence comprises a reporter and a binding site for an enzyme present in the body fluid sample, wherein the synthetic molecule reacts with the enzyme from the body fluid, causing the reporter to generate a detectable signal, and detecting the detectable signal.

In some cases, a linker of the plurality of linkers comprises a spacer coupled to an organic molecule. In some cases, the spacer comprises a PEG sequence. In some cases, the PEG sequence comprises a PEG2 sequence. In some cases, the organic molecule comprises an imide, a tetrazine, a cyclooctyne, an azide, an alkyne, or a phosphine. In some cases, the imide comprises formula (I):

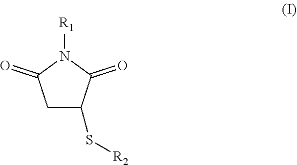

In some cases, the synthetic molecule further comprises an amine core. In some cases, the amine core comprises an ethylenediamine core or a polyamidoamine core. In some cases, the synthetic molecule is configured to detect activity of the enzyme. In some cases, the detectable signal is generated by an activity of the enzyme. In some cases, the enzyme activity comprises a disease-related enzyme activity, a baseline enzyme activity, or a combination thereof. In some cases, the synthetic molecule is configured to detect the enzyme activity in a proximal biofluid. In some cases, the detectable signal is generated by an enzyme activity in a proximal biofluid. In some cases, the enzyme activity indicates a presence of a pathogen, wherein the pathogen is associated with a disease.

In some cases, the reporter comprises a fluorescent molecule. In some cases, the fluorescent molecule comprises a FRET peptide. In some cases, the synthetic molecule further comprises a probe. In some cases, the probe is selected from Table 1. In some cases, the synthetic molecule further comprises an IEPD dendrimer, and the probe comprises a probe 9 molecule, a probe 102 molecule, a probe 379 molecule, or a combination thereof. In some cases, the synthetic molecule further comprises a plurality of probes. In some cases, the plurality of probes are selected from Table 1. In some cases, the dendrimer comprises an IPED dendrimer, and wherein the probe comprises a probe 102 molecule or a probe 379 molecule. In some cases, the IEPD dendrimer comprises a G4-IEPD dendrimer, a G5-IEPD dendrimer, a G6-IEPD dendrimer, a G7-IEPD dendrimer, a G8-IEPD dendrimer, a G9-IPED dendrimer, or a G10-IEPD dendrimer.

In some cases, the enzyme comprises a protease. In some cases, the protease is selected from the group consisting of an A20 (TNFa-induced protein 3), an abhydrolase domain containing 4, an abhydrolase domain containing 12, an abhydrolase domain containing 12B, an abhydrolase domain containing 13, an acrosin, an acylaminoacyl-peptidase, a disintegrin and metalloproteinase (ADAM), an ADAMla, an ADAM2 (Fertilin-b), an ADAM3B, an ADAM4, an ADAM4B, an ADAM5, an ADAM6, an ADAM7, an ADAM8, an ADAM9, an ADAM10, an ADAM11, an ADAM12 metalloprotease, an ADAM15, an ADAM17, an ADAM18, an ADAM19, an ADAM20, an ADAM21, an ADAM22, an ADAM23, an ADAM28, an ADAM29, an ADAM30, an ADAM32, an ADAM33, a disintegrin and metalloproteinase with thrombospondin motifs (ADAMTS), an ADAMTS1, an ADAMTS2, an ADAMTS3, an ADAMTS4, an ADAMTS5/11, an ADAMTS6, an ADAMTS7, an ADAMTS8, an ADAMTS9, an ADAMTS10, an ADAMTS12, an ADAMTS13, an ADAMTS14, an ADAMTS15, an ADAMTS16, an ADAMTS17, an ADAMTS18, an ADAMTS19, an ADAMTS20, an adipocyte-enhancer binding protein 1, an Afg3-like protein 1, an Afg3-like protein 2, an airway-trypsin-like protease, an aminoacylase, an aminopeptidase A, an aminopeptidase B, an aminopeptidase B-like 1, an aminopeptidase MAMS/L-RAP, an aminopeptidase N, an aminopeptidase O, an aminopeptidase P homologue, an aminopeptidase P1, an aminopeptidase PILS, an aminopeptidase Q, an aminopeptidase-like 1, an AMSH/STAMBP, an AMSH-LP/STAMBPL1, an angiotensin-converting enzyme 1 (ACE1), an angiotensin-converting enzyme 2 (ACE2), an angiotensin-converting enzyme 3 (ACE3), an anionic trypsin (II), an apolipoprotein (a), an archaemetzincin-1, an archaemetzincin-2, an aspartoacylase, an aspartoacylase-3, an aspartyl aminopeptidase, an ataxin-3, an ataxin-3 like, an ATP/GTP binding protein 1, an ATP/GTP binding protein-like 2, an ATP/GTP binding protein-like 3, an ATP/GTP binding protein-like 4, an ATP/GTP binding protein-like 5, an ATP23 peptidase, an autophagin-1, an autophagin-2, an autophagin-3, an autophagin-4, an azurocidin, a beta lactamase, a beta-secretase 1, a beta-secretase 2, a bleomycin hydrolase, a brain serine proteinase 2, a BRCC36 (BRCA2-containing complex, sub 3), a calpain, a calpain 1, a calpain 2, a calpain 3, a calpain 4, a calpain 5, a calpain 6, a calpain 7, a calpain 7-like, a calpain 8, a calpain 9, a calpain 10, a calpain 11, a calpain 12, a calpain 13, a calpain 14, a calpain 15 (Solh protein), a cysteine protease, a carboxypeptidase A1, a carboxypeptidase A2, a carboxypeptidase A3, a carboxypeptidase A4, a carboxypeptidase A5, a carboxypeptidase A6, a carboxypeptidase B, a carboxypeptidase D, a carboxypeptidase E, a carboxypeptidase M, a carboxypeptidase N, a carboxypeptidase O, a carboxypeptidase U, a carboxypeptidase X1, a carboxypeptidase X2, a carboxypeptidase Z, a carnosine dipeptidase 1, a carnosine dipeptidase 2, a caspase recruitment domain family, member 8, a caspase, a caspase-1, a caspase-2, a caspase-3, a caspase-4/11, a caspase-5, a caspase-6, a caspase-7, a caspase-8, a caspase-9, a caspase-10, a caspase-12, a caspase-14, a caspase-14-like, a casper/FLIP, a cathepsin, a cathepsin A (CTSA), a cathepsin B (CTSB), a cathepsin C (CTSC), a cathepsin D (CTSD), a cathepsin E (CTSE), a cathepsin F, a cathepsin G, a cathepsin H (CTSH), a cathepsin K (CTSK), a cathepsin L (CTSL), a cathepsin L2, a cathepsin O, a cathepsin S (CTSS), a cathepsin V (CTSV), a cathepsin W, a cathepsin Z (CTSZ), a cationic trypsin, a cezanne/OTU domain containing 7B, a cezanne-2, a CGI-58, a chymase, a chymopasin, a chymosin, a chymotrypsin B, a chymotrypsin C, a coagulation factor IXa, a coagulation factor VIIa, a coagulation factor Xa, a coagulation factor XIa, a coagulation factor XIIa, a collagenase 1, a collagenase 2, a collagenase 3, a complement protease C1r serine protease, a complement protease C1s serine protease, a complement C1r-homolog, a complement component 2, a complement component C1ra, a complement component Csa, a complement factor B, a complement factor D, a complement factor D-like, a complement factor I, a COPS6, a corin, a CSN5 (JAB1), a cylindromatosis protein, a cytosol alanyl aminopep.-like 1, a cytosol alanyl aminopeptidase, a DDI-related protease, a DECYSIN, a Der1-like domain family, member 1, a Der1-like domain family, member 2, a Der1-like domain family, member 3, a DESC1 protease, a desert hedgehog protein, a desumoylating isopeptidase 1, a desumoylating isopeptidase 2, a dihydroorotase, a dihydropyrimidinase, a dihydropyrimidinase-related protein 1, a dihydropyrimidinase-related protein 2, a dihydropyrimidinase-related protein 3, a dihydropyrimidinase-related protein 4, a dihydropyrimidinase-related protein 5, a DINE peptidase, a dipeptidyl peptidase (DPP), a dipeptidyl peptidase (DPP1), a dipeptidyl-peptidase 4 (DPP4), a dipeptidyl-peptidase 6 (DPP6), a dipeptidyl-peptidase 8 (DPP8), a dipeptidyl-peptidase 9 (DPP9), a dipeptidyl-peptidase II, a dipeptidyl-peptidase III, a dipeptidyl-peptidase 10 (DPP10), a DJ-1, a DNA-damage inducible protein, a DNA-damage inducible protein 2, a DUB-1, a DUB-2, a DUB2a, a DUB2a-like, a DUB2a-like2, a DUB6, or a combination thereof.

In some cases, the protease is selected from the group consisting of a T-cell protease, a complement protease, a fibrosis protease, and an inflammation-related protease. In some cases, the synthetic molecule further comprises a carrier. In some cases, the carrier comprises a native, labeled or synthetic protein, a synthetic chemical polymer of precisely known chemical composition or with a distribution around a mean molecular weight, an oligonucleotide, a phosphorodiamidate morpholino oligomer (PMO), a foldamer, a lipid, a lipid micelle, a nanoparticle, a solid support comprising polystyrene, polypropylene or any other type of plastic compound, or any combination thereof. In some cases, the plurality of linkers comprises a peptide, a carbohydrate, a nucleic acid, a lipid, an ester, a glycoside, a phospholipid, a phosphodiester, a nucleophile/base sensitive linker, a reduction sensitive linker, an electrophile/acid sensitive linker, a metal cleavable linker, an oxidation sensitive linker, or a combination thereof. In some cases, the synthetic molecule is present in the body fluid sample at a concentration of approximately 0.01 nM-0.1M. In some cases, the enzyme is present in the body fluid sample at a concentration of approximately 0.01 nM-1.0 nM.

In some cases, the detectable signal is generated when the enzyme is present in the body fluid sample at a concentration of approximately 0.01 nM to approximately 1.0 nM. In some cases, the peptide sequence is configured to have an increased affinity to an enzyme compared to a linear peptide sequence that is not coupled to the dendrimer. In some cases, the synthetic molecule further comprising a plurality of peptide sequences. In some cases, the plurality of peptide sequences comprises approximately 1-50 peptides, 50-100 peptides, or 100-150 peptides. In some cases, plurality of peptide sequences are different than one another, similar to each other, or a combination thereof. In some cases, the at least one peptide sequence comprises a tunable sequence. In some cases, the synthetic molecule is configured to react with a paper strip application. In some cases, further comprising detecting a rate of generation or an amount of the detectable signal.

In another aspect, provided herein is a method of synthesizing a molecule, comprising providing linker components comprising an organic molecule and an inert spacer, thereby producing a linker, providing a synthetic polymer comprising a core, a plurality of branch points, a plurality of end points, and a free amino group, providing a peptide with a free thiol group, wherein the linker couples to the synthetic polymer via the plurality of endpoints, and wherein the organic molecule reacts with the free thiol group, thereby covalently binding the peptide to the linker.

In some cases, the organic molecule comprises an imide, a maleimide, a tetrazine, a cyclooctyne, an azide, an alkyne, or a phosphine. In some cases, the imide comprises formula (I):

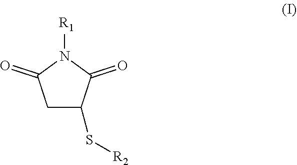

In some cases, the organic molecule comprises an N-hydroxysuccinimide (NHS), a maleimide, or a combination thereof. In some cases, the inert spacer comprises a PEG sequence. In some cases, the synthetic molecule comprises an IPED dendrimer and a probe 102 molecule or a probe 379 molecule. In some cases, the IEPD dendrimer comprises a G4-IEPD dendrimer, a G5-IEPD dendrimer, a G6-IEPD dendrimer, a G7-IEPD dendrimer, a G8-IEPD dendrimer, a G9-IPED dendrimer, or a G10-IEPD dendrimer. In some cases, the core comprises an ethylenediamine core or a polyamidoamine core. In some cases, the peptide comprises a sequence having a binding site for an enzyme.

In some cases, the enzyme comprises a protease. In some cases, the protease is selected from the group consisting of an A20 (TNFa-induced protein 3), an abhydrolase domain containing 4, an abhydrolase domain containing 12, an abhydrolase domain containing 12B, an abhydrolase domain containing 13, an acrosin, an acylaminoacyl-peptidase, a disintegrin and metalloproteinase (ADAM), an ADAM1a, an ADAM2 (Fertilin-b), an ADAM3B, an ADAM4, an ADAM4B, an ADAM5, an ADAM6, an ADAM7, an ADAM8, an ADAM9, an ADAM10, an ADAM11, an ADAM12 metalloprotease, an ADAM15, an ADAM17, an ADAM18, an ADAM19, an ADAM20, an ADAM21, an ADAM22, an ADAM23, an ADAM28, an ADAM29, an ADAM30, an ADAM32, an ADAM33, a disintegrin and metalloproteinase with thrombospondin motifs (ADAMTS), an ADAMTS1, an ADAMTS2, an ADAMTS3, an ADAMTS4, an ADAMTS5/11, an ADAMTS6, an ADAMTS7, an ADAMTS8, an ADAMTS9, an ADAMTS10, an ADAMTS12, an ADAMTS13, an ADAMTS14, an ADAMTS15, an ADAMTS16, an ADAMTS17, an ADAMTS18, an ADAMTS19, an ADAMTS20, an adipocyte-enhancer binding protein 1, an Afg3-like protein 1, an Afg3-like protein 2, an airway-trypsin-like protease, an aminoacylase, an aminopeptidase A, an aminopeptidase B, an aminopeptidase B-like 1, an aminopeptidase MAMS/L-RAP, an aminopeptidase N, an aminopeptidase O, an aminopeptidase P homologue, an aminopeptidase P1, an aminopeptidase PILS, an aminopeptidase Q, an aminopeptidase-like 1, an AMSH/STAMBP, an AMSH-LP/STAMBPL1, an angiotensin-converting enzyme 1 (ACE1), an angiotensin-converting enzyme 2 (ACE2), an angiotensin-converting enzyme 3 (ACE3), an anionic trypsin (II), an apolipoprotein (a), an archaemetzincin-1, an archaemetzincin-2, an aspartoacylase, an aspartoacylase-3, an aspartyl aminopeptidase, an ataxin-3, an ataxin-3 like, an ATP/GTP binding protein 1, an ATP/GTP binding protein-like 2, an ATP/GTP binding protein-like 3, an ATP/GTP binding protein-like 4, an ATP/GTP binding protein-like 5, an ATP23 peptidase, an autophagin-1, an autophagin-2, an autophagin-3, an autophagin-4, an azurocidin, a beta lactamase, a beta-secretase 1, a beta-secretase 2, a bleomycin hydrolase, a brain serine proteinase 2, a BRCC36 (BRCA2-containing complex, sub 3), a calpain, a calpain 1, a calpain 2, a calpain 3, a calpain 4, a calpain 5, a calpain 6, a calpain 7, a calpain 7-like, a calpain 8, a calpain 9, a calpain 10, a calpain 11, a calpain 12, a calpain 13, a calpain 14, a calpain 15 (Solh protein), a cysteine protease, a carboxypeptidase A1, a carboxypeptidase A2, a carboxypeptidase A3, a carboxypeptidase A4, a carboxypeptidase A5, a carboxypeptidase A6, a carboxypeptidase B, a carboxypeptidase D, a carboxypeptidase E, a carboxypeptidase M, a carboxypeptidase N, a carboxypeptidase O, a carboxypeptidase U, a carboxypeptidase X1, a carboxypeptidase X2, a carboxypeptidase Z, a carnosine dipeptidase 1, a carnosine dipeptidase 2, a caspase recruitment domain family, member 8, a caspase, a caspase-1, a caspase-2, a caspase-3, a caspase-4/11, a caspase-5, a caspase-6, a caspase-7, a caspase-8, a caspase-9, a caspase-10, a caspase-12, a caspase-14, a caspase-14-like, a casper/FLIP, a cathepsin, a cathepsin A (CTSA), a cathepsin B (CTSB), a cathepsin C (CTSC), a cathepsin D (CTSD), a cathepsin E (CTSE), a cathepsin F, a cathepsin G, a cathepsin H (CTSH), a cathepsin K (CTSK), a cathepsin L (CTSL), a cathepsin L2, a cathepsin O, a cathepsin S (CTSS), a cathepsin V (CTSV), a cathepsin W, a cathepsin Z (CTSZ), a cationic trypsin, a cezanne/OTU domain containing 7B, a cezanne-2, a CGI-58, a chymase, a chymopasin, a chymosin, a chymotrypsin B, a chymotrypsin C, a coagulation factor IXa, a coagulation factor VIIa, a coagulation factor Xa, a coagulation factor XIa, a coagulation factor XIIa, a collagenase 1, a collagenase 2, a collagenase 3, a complement protease C1r serine protease, a complement protease C1s serine protease, a complement C1r-homolog, a complement component 2, a complement component C1ra, a complement component C1sa, a complement factor B, a complement factor D, a complement factor D-like, a complement factor I, a COPS6, a corin, a CSN5 (JAB1), a cylindromatosis protein, a cytosol alanyl aminopep.-like 1, a cytosol alanyl aminopeptidase, a DDI-related protease, a DECYSIN, a Der1-like domain family, member 1, a Der1-like domain family, member 2, a Der1-like domain family, member 3, a DESC1 protease, a desert hedgehog protein, a desumoylating isopeptidase 1, a desumoylating isopeptidase 2, a dihydroorotase, a dihydropyrimidinase, a dihydropyrimidinase-related protein 1, a dihydropyrimidinase-related protein 2, a dihydropyrimidinase-related protein 3, a dihydropyrimidinase-related protein 4, a dihydropyrimidinase-related protein 5, a DINE peptidase, a dipeptidyl peptidase (DPP), a dipeptidyl peptidase (DPP1), a dipeptidyl-peptidase 4 (DPP4), a dipeptidyl-peptidase 6 (DPP6), a dipeptidyl-peptidase 8 (DPP8), a dipeptidyl-peptidase 9 (DPP9), a dipeptidyl-peptidase II, a dipeptidyl-peptidase III, a dipeptidyl-peptidase 10 (DPP10), a DJ-1, a DNA-damage inducible protein, a DNA-damage inducible protein 2, a DUB-1, a DUB-2, a DUB2a, a DUB2a-like, a DUB2a-like2, a DUB6, or a combination thereof.

In some cases, the protease is selected from the group consisting of a T cell protease, a complement protease, a fibrosis protease, and an inflammation-related protease. In some cases, an NHS group reacts with the free amino group, thereby covalently binding the linker to the synthetic polymer. In some cases, the synthetic polymer comprises a dendrimer, a multivalent synthetic macromolecule, a nanoparticle scaffold, a polymeric scaffold, a foldamer, a branched peptide, or a synthetic composite nanoparticle. In some cases, the synthetic polymer further comprises a probe. In some cases, the probe is selected from Table 1. In some cases, the synthetic polymer comprises an IEPD dendrimer, and wherein the probe comprises a probe 9 molecule, a probe 102 molecule, or a probe 379 molecule, or a combination thereof. In some cases, the synthetic polymer further comprises a plurality of probes. In some cases, the plurality of probes are selected from Table 1.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (“FIGURE.”, “FIG.” or “FIGURES.”, “FIGs.” herein), of which:

FIGS. 1A and 1B illustrate a method for synthesizing a synthetic molecule 100 comprising a plurality of linkers 110 coupled to a synthetic polymer 101, each of the plurality of linkers comprising a first end 105, a second end 106, and a linker spacer sequence, wherein the second end comprises a reactive handle 141 (FIG. 1A), and a method of contacting the synthetic molecule 100 to a body fluid sample, wherein the body fluid sample comprises an enzyme 150 (FIG. 1B).

FIGS. 2A-C illustrate a synthetic polymer comprising a plurality of linkers 210, each of the plurality of linkers comprising a reactive handle 241 (FIG. 2A), an exemplary synthetic polymer 201 (FIG. 2B), and a synthetic polymer 201 comprising a core 202, branch points 203, and end points 204 (FIG. 2C). In some cases, the end points 204 comprise a surface or surface groups to which an end of each of a plurality of linkers conjugates.

FIG. 3 depicts a synthetic molecule 300, comprising a synthetic polymer 301, a linker 310 comprising a linker spacer sequence 307 and a reactive handle 341, wherein the linker 310 is coupled to a peptide sequence 315 via the reactive handle 341.

FIG. 4 depicts an exemplary method for synthesizing a synthetic molecule, in accordance with an embodiment of the present disclosure.

FIG. 5 depicts an exemplary method for coupling a linker via a functional group to a reactive handle of a synthetic polymer.

FIG. 6 depicts an exemplary reaction for coupling a peptide sequence to a linker functionalized polymer.

FIG. 7 is a graphical representation of a purification of a synthetic molecule to remove excess unconjugated peptide for determining the concentration of a synthetic molecule.

FIG. 8 is a graphical representation of quantifying a molar amount of a linear peptide conjugated to a dendrimer and of a linear peptide not conjugated to a dendrimer in a buffer solution not containing an enzyme and in a buffer solution containing an enzyme.

FIGS. 9A-B are graphical representations of Michaelis-Menten kinetics of a solution containing a linear molecule (FIG. 9A) and a synthetic molecule (FIG. 9B).

FIGS. 10A-B are graphs showing a limit of detection or sensitivity of a linear molecule (FIG. 10A) and a synthetic molecule (FIG. 10B) in a buffer-based solution.

FIGS. 11A-B are graphs showing a limit of detection or sensitivity of a linear molecule (FIG. 11A) and a synthetic molecule (FIG. 11B) in an alternative biofluid.

FIGS. 12A-B are graphs showing a level of detection of a linear molecule and a synthetic molecule over a period of at least 3 hours in a sample comprising an agent, wherein said agent is present at a concentration of 1% (FIG. 12A) and 0.1% (FIG. 12B), limit of detection or sensitivity of a linear molecule (FIG. 12A) and a synthetic molecule (FIG. 12B) in a buffer-based solution.

FIG. 13 is a graphical representation of a quantification of a molar amount of a peptide conjugated to a dendrimer and a molar amount of the peptide not conjugated to a dendrimer after reaction with a specific enzyme.

FIGS. 14A-B are graphical representations of Michaelis Menten enzyme kinetics of a peptide not conjugated to a dendrimer at varying concentrations (FIG. 14A) and of Michaelis Menten enzyme kinetics of a peptide conjugated to a dendrimer at varying concentrations (FIG. 14B).

FIGS. 15A-B are graphical representations of a limit of detection experiment for detecting levels of a linear peptide (FIG. 15A) and for detecting levels of a peptide conjugated to a dendrimer (FIG. 15B) in a buffer-based system.

FIGS. 16A-B are graphical representations of a limit of detection experiment detecting a peptide (FIG. 16A) and a peptide conjugated to a dendrimer (FIG. 16B) in a body fluid sample.

FIGS. 17A-B are graphical representations showing spectral properties of a peptide in a solution at varying concentrations (FIG. 17A) and of a peptide conjugated to a dendrimer following purification from a solution (FIG. 17B).

### DETAILED DESCRIPTION

Provided herein are methods comprising contacting a body fluid sample from a subject with a synthetic molecule, methods for producing a synthetic molecule, and a synthetic molecule. In some embodiments, the synthetic molecule comprises a linker and a reporter, and the linker is cleaved by an agent contained in the body fluid, thereby releasing the reporter from the molecule. In some embodiments, the released reporter generates a detectable signal. The strength of the signal, according to some embodiments, indicates a presence or an occurrence of a disease or a condition. In some embodiments, the method comprises detecting a rate of formation or an amount the released reporter. In some embodiments, the rate of formation or amount of the released reporter in a subject suspected of having said condition or disease is significantly different than the rate of formation or amount of the released reporter in a second subject.

### Synthetic Molecule

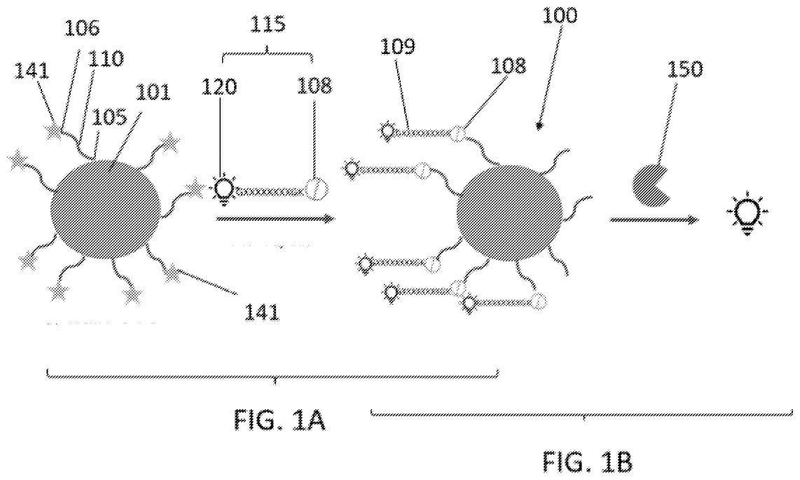

As illustrated by FIGS. 1A-B, provided herein is a synthetic molecule 100 comprising a synthetic polymer 101, a plurality of linkers 110, and a peptide sequence 115. Each of the plurality of linkers is coupled to a surface or a surface group of the synthetic polymer. In some cases, each of the plurality of linkers 110 comprises a linker spacer sequence, a first end 105 and a second end 106. In some cases, the linker comprises a reactive handle 141 configured to react with a click handle on a peptide sequence. In some cases, the peptide sequence 115 comprises a click handle, a peptide sequence spacer 109, and a reporter 120. As disclosed herein, a click handle refers to a molecule, compound comprising a molecule, or chemical moiety that reacts with another click handle or a reactive handle, wherein the another click handle or the reactive handle also comprises a molecule, a compound comprising a molecule, or a chemical moiety. In some cases, the peptide sequence comprises a peptide spacer sequence 109, a reporter 120, and a quencher 108. In some cases, the first end 105 or second end 106 of the linker comprises a reactive handle 141. In some cases, the first end 105 or the second end 106 of the linker comprises a surface group. In some cases, the click handle is configured to react with the reactive handle 141 of the linker 110 coupled to the synthetic polymer 101. In some aspects, the linker disclosed herein is bifunctional. In some aspects, the peptide sequence is bifunctional. In some aspects, the linker and the peptide sequence are bifunctional. In some cases, the peptide sequence 115 has a binding site for an enzyme 150. In some cases, the enzyme 150 binds to the binding site on the peptide sequence 115, thereby releasing a reporter 120 coupled to the peptide sequence 115.

A “synthetic molecule,” as disclosed herein, refers to a particle conjugated to a substrate for bioactivity detection in biofluids, bio-samples, and environments. In some cases, the environment comprises an ex vivo environment. In some cases, the ex vivo environment comprises a sample obtained from a subject. In some cases, the environment comprises an in vivo environment. In some cases the environment comprises an in vitro environment. In some cases, the subject is a human. In some cases, the environment comprises an in vivo environment. Some example embodiments of an in vivo environment include a tissue or a fluid in or of a subject. In some cases, the subject is a human. The substrate conjugated to the particle is configured to be released in an environment in response to the presence of an agent in the environment. The release of the substrate from the particle generates a detectable signal that, when detected, quantifies a level of activity of the agent, an amount of the agent that is present in the sample or the environment, a rate of formation of detectable signal, or a combination thereof. In some cases, the level of activity of the agent indicates a presence of a disease or condition, or a stage of a disease or condition, in the environment.

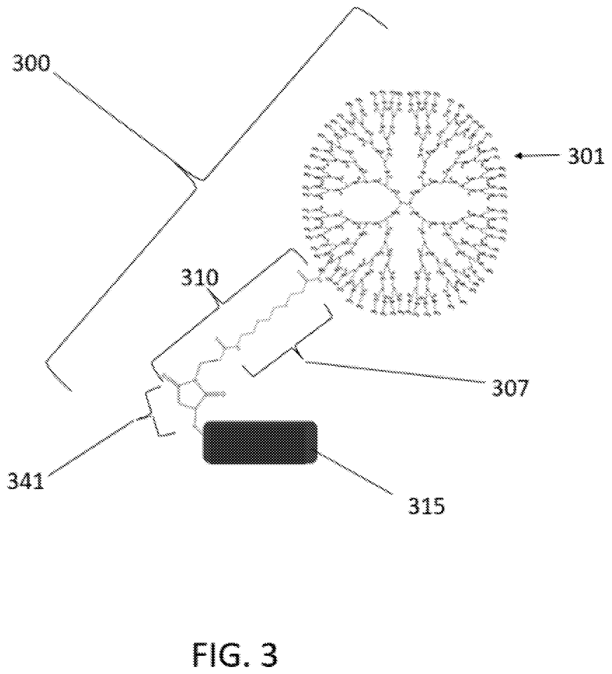

As depicted in FIG. 3, an embodiment of the present disclosure provides a synthetic molecule 300 comprising a synthetic polymer 301 and a linker 310 coupled to a peptide sequence 315. In some embodiments, the linker comprises a linker spacer sequence 307 and a reactive handle 341, wherein the linker 310 is coupled to the peptide sequence 315 via the reactive handle 341. In some cases, the synthetic molecule comprises a plurality of substrates. In some cases, each of the plurality of substrates is conjugated to the synthetic molecule via a linker. In some cases, the linker comprises a plurality of linkers. In some cases, each linker, or each of the plurality of linkers, comprises a binding site for an agent, wherein the binding site on each linker is for an agent. In some cases, the plurality of linkers comprises a binding site having an affinity to a similar agent present in an environment. In some cases, the plurality of linkers comprises a binding site having an affinity for different agents present in an environment. In some cases, the environment comprises a subject. In some cases, the subject comprises a human. The particle comprises a plurality of end points comprising surface groups, to which the substrates are conjugated. In some cases, the substrates are conjugated to the particle via a linker. In some cases, the synthetic molecule is configured to detect a plurality of agents in an environment. Detecting a plurality of agents in an environment enables high throughput and efficient diagnosing of a disease or condition in a subject, determining of a stage of a disease or condition in a subject, or a combination thereof. In some example embodiments, a synthetic molecule comprises a dendrimer. In some cases, the dendrimer comprises between approximately 100-5000 surface groups, also referred herein to as endpoints. A greater amount of surface groups allows for highly specific and sensitive detection of one or more agent in an environment. In some cases, the synthetic molecule comprises a synthetic probe. In some embodiments, the synthetic probe is selected from Table 1.

A “synthetic polymer,” as disclosed herein, is defined as a scaffold or architectural motif comprising a core, a plurality of branch points, a plurality of end points. In some cases, the synthetic polymer comprises a dendrimer. Dendrimers are formed via successive addition of layers to branching groups, wherein each additional layer is referred to as a generation (e.g., generation 1 (G1), G2, G3, G4, G5, G6, etc.). In some cases, the dendrimer comprises a G1 dendrimer, a G2 dendrimer, a G3 dendrimer, a G4 dendrimer, a G5 dendrimer, a G6 dendrimer, a G7 dendrimer, a G8 dendrimer, a G9 dendrimer, or a G10 dendrimer. In some cases, the dendrimer comprises a polyamidoamine dendrimer, a polypropylene imine dendrimer, a polyethercopolyester dendrimer, or a polyethyleneglycolated (PEGylated) dendrimer. A dendrimer's final layer, or final generation, comprises additional active groups, also referred to as surface groups (e.g., endpoints), that comprise a particular functionality (e.g., reactivity with another molecule or molecular compound). In some cases, the synthetic polymer is radially symmetric with a multi-dispersed structure. In some cases, the synthetic polymer is radially symmetric with a monodispersed structure. In some embodiments disclosed herein, chemical and physical properties of a dendrimer are configurable or tunable. The chemical and physical properties of dendrimers depend on the molecules comprising the core, the plurality of branch points, and the plurality of end points. In some cases, the synthetic molecule disclosed herein comprises a dendrimer comprising a tunable design, wherein the tunable design enables a user to alter the molecular makeup or composition, and, as a result of altering the molecular makeup or composition, alter the chemical and physical properties, of the dendrimer.

A “linker” or a “plurality of linkers,” as disclosed herein, comprises a series of components, wherein the components comprise reactive components and nonreactive components. A reactive component, in some example cases, comprises a sequence or chemical formula configured to react with an agent in an environment, generate a detectable signal, or a combination thereof. In some cases, a reactive component comprises a site to which an agent binds, wherein the agent is present in an environment. According to some example embodiments, a reactive component comprises a substrate configured to be released in an environment, whereupon the release of the substrate in the environment generates a detectable signal. In some cases, the substrate is a reporter. In some cases, the detectable signal quantifies a level of activity. In some cases, the level of activity indicates a presence of a disease or a condition in an environment, a stage of the disease or the condition in the environment, or a combination thereof. In some cases, the environment is a subject. In some cases, the subject is a human. A non-reactive component, in some example cases, comprises an inert spacer. In some cases, the inert spacer comprises a hydrocarbon chain, a peptide sequence, a bifunctional organic molecule, a polyethylene glycol (PEG) chain, or a combination thereof. In some cases, the spacer comprises an aminohexanoic acid or a derivative thereof. In some embodiments, the PEG chain comprises configurable components. In some embodiments, the inert spacer is configured to prevent degradation of the synthetic molecule, to which the spacer is conjugated. In some cases, the non-reactive components are conjugated to a surface group, or an end point, via a first end. In some cases, the non-reactive components are conjugated to an organic molecule via a second end. The organic molecule is configured to connect the inert spacer to the peptide sequence coupled to a reporter, wherein the reporter generates a detectable signal upon release of the reporter from the peptide sequence coupled to the organic molecule. In some cases, the organic molecule comprises an imide, a tetrazine, a cyclooctyne, an azide, an alkyne, a phosphine, a norbornene, a thiol, an alkyne, an aldehyde, a hydroxylamine, a diene, a dienophile, a hydroxysuccinimide, or an amine.

In some cases, linkers are coupled to a peptide sequence on an end and to surface groups on another end. In some cases, the surface groups are monodispersed on a synthetic polymer. In some cases, the surface groups are multi-dispersed on a synthetic polymer. In some embodiments, the linkers comprise a first end and a second end. In some cases, the first end of the linker is coupled to an endpoint and the second end is coupled to a peptide sequence. In some cases, the peptide sequence is coupled to a reporter via a sequence susceptible to cleavage by an agent 150. In some cases, the agent 150 is an enzyme. In some embodiments, the enzyme is a protease.

In some cases, the synthetic molecule is configured to detect a protease activity. The protease activity indicates a presence or an occurrence of a disease or condition known to be associated with the detected protease activity. In some cases, the protease activity comprises a disease-related protease activity, a baseline protease activity, or a combination thereof. In some cases, the protease activity indicates a presence of a pathogen, wherein said pathogen causes a disease.

In some cases, the disease or condition comprises a liver disease, a cancer, a metabolic disease, a fibrotic disease, an organ transplant rejection, an infectious disease, an allergic disease, an autoimmunity, Alzheimer's or a chronic inflammation. In some embodiments, the liver disease may be a non-alcoholic steatohepatitis (NASH), a non-alcoholic fatty liver disease (NAFLD), a toxin mediated liver injury (drug/medication, alcohol, environmental), a viral hepatitis (HAV, HBV, HCV, HDV, HEV, other virus infecting the liver), an autoimmune hepatitis, a primary biliary cholangitis, a primary sclerosing cholangitis, a fulminant hepatitis, a cirrhosis of the liver, a hepatocellular carcinoma (HCC), a cholangiocarcinoma, an acute or chronic rejection of a transplanted liver, an inherited liver disease (e.g. Wilson disease, hemochromatosis, or alpha-1 antitrypsin) or a combination thereof.

### Synthetic Polymer

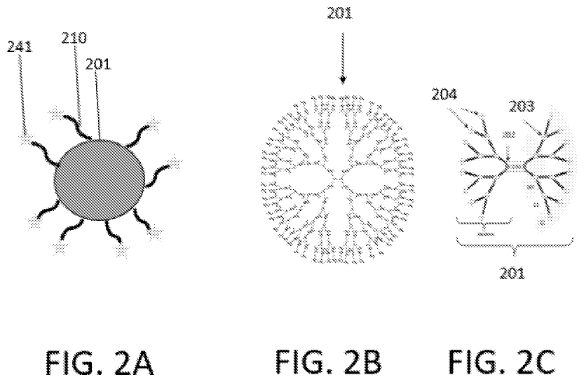

In some cases, a synthetic molecule comprises a synthetic polymer. As seen in FIGS. 2A-C, the synthetic polymer 201 comprises a core 202, a plurality of branch points 203, a plurality of end points 204 comprising a plurality of surface groups, coupled to a plurality of linkers 210. In some cases, each of the plurality of linkers comprises a reactive handle 241.

In some cases, the synthetic polymer comprises a dendrimer. In some cases, the synthetic polymer comprises a spheroid polymeric nanostructure that repeatedly branches outward from an inner core. In some embodiments, the core is multimeric.

In some cases, the synthetic polymer comprises a three-dimensional molecular framework. The highly branched architecture comprising a molecular framework of the synthetic polymer is configured to allow for a greater number of surface groups, configured such that diagnostic and therapeutic agents attach to the surface groups. In some cases, the synthetic polymer can have a plurality of surface groups, to which a plurality of different linkers bind. In some cases, said each of the plurality of different linkers comprises a different organic molecule coupled to a peptide sequence, wherein the peptide sequence is coupled to a reporter and wherein the peptide sequence is susceptible to cleavage by an agent present in an environment.

In some cases, the synthetic polymer comprises a plurality of branch points. Branch points play a role in the functioning of the synthetic polymer and determine branching generations. The higher the number of surface groups, the higher the generation number (32 surface groups=G3; 64 surface groups=G4; 128 surface groups=G5; 256 surface groups=G6; 512 surface groups=G7; 1024 surface groups=G8; 2048 surface groups=G9; 4096 surface groups=G10; and so forth). In some cases, the synthetic polymer comprises a dendrimer. In some cases, the dendrimer comprises a G3 dendrimer, a G4 dendrimer, a G5 dendrimer, a G6 dendrimer, a G7 dendrimer, a G8 dendrimer, a G9 dendrimer, a G10 dendrimer, a G11 dendrimer, a G12 dendrimer, or a G13 dendrimer. Using or introducing a dendrimer comprising more branch points results in a high number of surface groups, providing for a commercially available tunable design, capable of having up to approximately 6000 surface groups.

In some cases, the synthetic polymer comprises a dendrimer, a multivalent synthetic macromolecule, a nanoparticle scaffold, a polymeric scaffold, a foldamer, a branched peptide, or a synthetic composite nanoparticle.

### Organic Molecule

In some cases, the synthetic molecule disclosed herein comprises a reactive handle. In some cases, the synthetic molecule disclosed herein comprises a click handle. In some cases, the reactive handle and/or the click handle comprise an organic molecule. In some cases, the organic molecule comprises a tetrazine, a cyclooctyne, an azide, an alkyne, a phosphine, a norbornene, a thiol, an alkene, an aldehyde, a hydroxylamine, a diene, a dienophile, a hydroxysuccinimide, or an amine.

In some cases, the organic molecule comprises a reactive moiety. In some cases, the reactive moiety comprises thiols, alkenes, alkynes, imides, disulfides, perfluoro-arylated molecules, decafluorobiphenyl, hexafluorobenzene, or a combination thereof. In some cases, the organic molecule is configured to undergo a click chemistry reaction. Click chemistry reactions are high yielding reactions that create byproducts that are removable without chromatography, are stereospecific, simple to perform, and can be conducted in benign solvents. In some cases, the organic molecule comprises a tunable region that facilitates binding of the organic molecule to a peptide sequence.

### Peptide Sequence

In some cases, the synthetic molecule disclosed herein comprises a peptide sequence. In some cases, the peptide sequence is coupled to a linker conjugated to a surface or a surface group of a synthetic polymer disclosed herein. In some cases, the peptide sequence comprises a reporter, a peptide sequence spacer, and a surface group. In some aspects, the peptide sequence is configured to react with a detection scheme. In some cases, the reporter is configured to react with a detection scheme.

In some cases, the peptide sequence comprises at least one click handle. In some cases, the at least one click handle is configured to react with a linker conjugated to a synthetic polymer, thereby coupling the peptide sequence to the linker. In some cases, the peptide sequence comprises a click handle. In some cases, the at least one click handle comprises an organic compound comprising a functional group. In some cases, the functional group comprises the chemical formula R—X, wherein X is the functional group (e.g., R—SH).

### Method of Synthesizing a Molecule

The present disclosure comprises a method for synthesizing a molecule comprising providing linker components, providing a synthetic polymer, and providing a peptide with a free thiol group. In some embodiments, linker components comprise at least one organic compound and an inert spacer. In some embodiments, the synthetic polymer comprises a core, a plurality of branch points, and a plurality of end points, wherein the synthetic polymer further comprises a free amino group. In some embodiments, an NHS group reacts with the free amino acid group so that the linker is covalently bound to the scaffold.

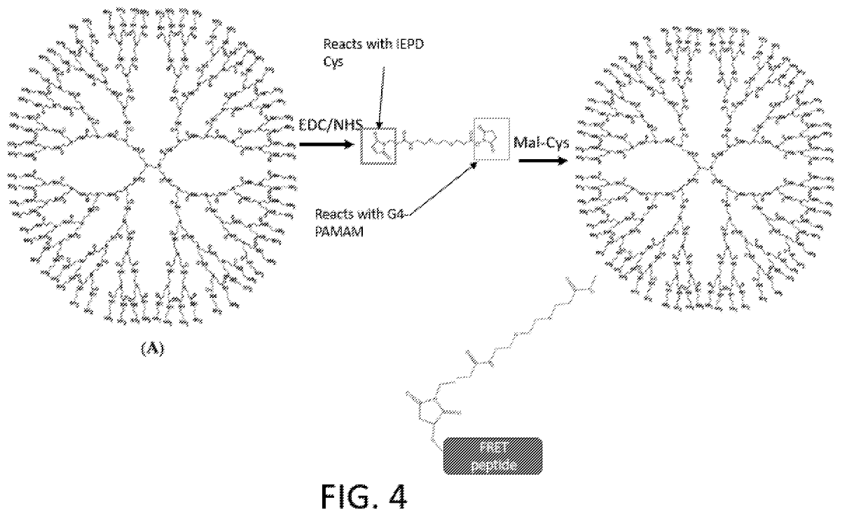

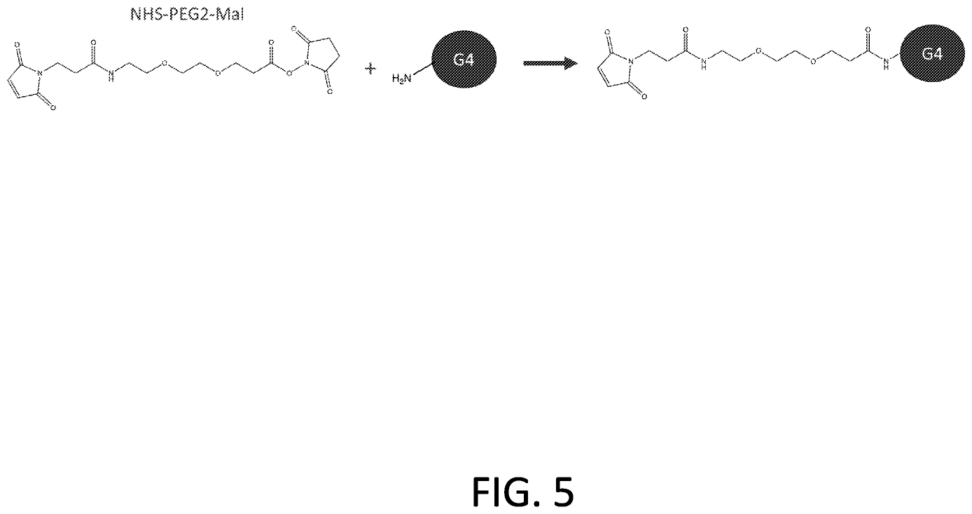

As exemplified by FIG. 4, and as reflected in some embodiments disclosed herein, a synthetic polymer and a linker comprising a first end and a second end are provided. In some embodiments, the linker comprises a first end and a second end. Some embodiments comprise handles, which correspond to the first end and the second end. The click pairs are invertedly on the synthetic polymer or on the organic molecule. The first end of the linker couples to a surface group on the synthetic polymer, and the second end of the linker couples to a peptide sequence. In some embodiments, the second end of the linker comprises an organic molecule, which is coupled to a spacer and to the peptide sequence. In some embodiments, the spacer is an inert spacer. In some embodiments, the surface group is a primary amine group comprising the chemical formula R—NH2, as shown in FIG. 5. Linkers disclosed herein, in some cases, are bifunctional. Bifunctional linkers are configured to have two reactive handles, enabling the linker to react with more than one compound. In some example embodiments, a linker comprises a first end configured to react with an end point on a synthetic polymer and a second end comprising a reactive handle configured to react with a click handle on a peptide sequence. In some cases, the click handle is located on an end of a peptide sequence. In some cases, the click handle is located along the peptide sequence. After the linker couples to the synthetic polymer, the product is purified via MWCO 10,000 kDa.

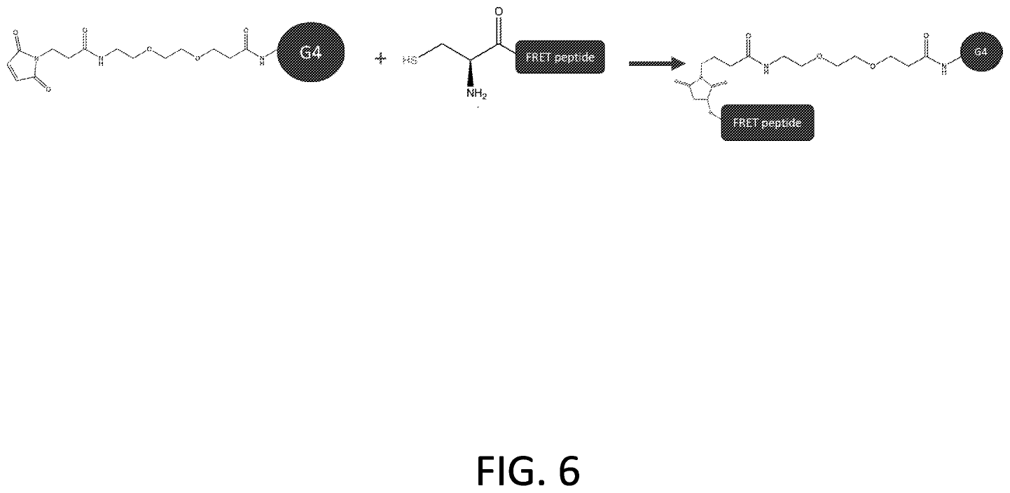

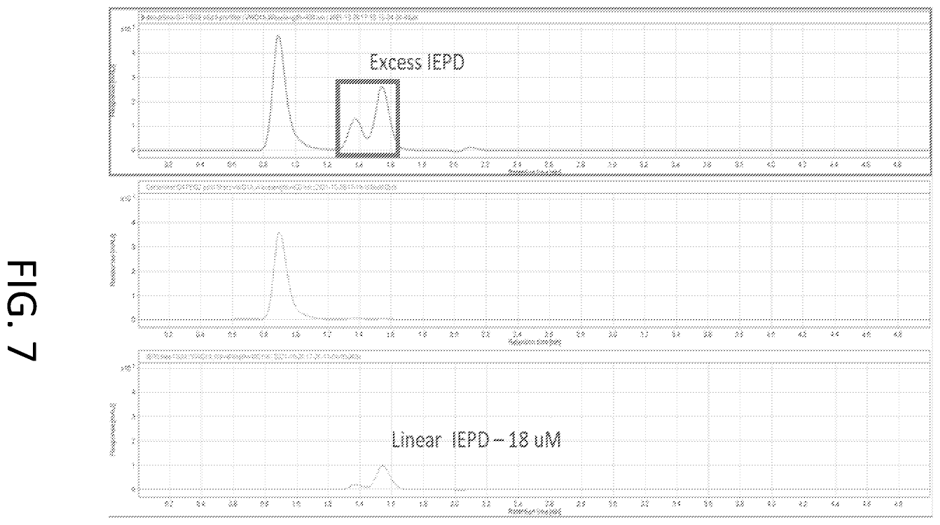

As shown in FIG. 6, following the coupling of the linker to the synthetic polymer, a peptide sequence is provided. The peptide sequence reacts with the synthetic polymer coupled to the linker at a second end of the linker. In some embodiments, the second end of the linker comprises an organic molecule having an oxygen atom, wherein said oxygen atom reacts with a thiol group coupled to a peptide sequence, yielding a synthetic polymer coupled to a linker via a first end, wherein the linker is coupled to the peptide sequence via an organic molecule coupled to the second end of the linker. In some embodiments, the organic molecule that reacts with the thiol group to couple the peptide sequence to the linker coupled to the synthetic polymer comprises an imide, a tetrazine, a cyclooctyne, an azide, an alkyne, a phosphine, a norbornene, a thiol, an alkene, an aldehyde, a hydroxylamine, a diene, a dienophile, a hydroxysuccinimide, or an amine

Synthetic polymers described herein are functionalized. In some cases, the synthetic polymers encapsulate other molecules in their interior voids. In some cases, the synthetic polymers comprise molecules attached to the synthetic polymer's periphery. In some cases, the synthetic polymers are self-assembling. In some cases, the synthetic polymers comprise endpoints comprising a reactive group, wherein the reactive group is configured to react with a reactive handle on a linker, thereby coupling the linker to the synthetic polymer.

### Carrier

In some aspects, the synthetic molecule described herein comprises a carrier. In some aspects, the carrier is a vesicle. In some cases, the vesicle is configured to contain at least one synthetic molecule. In some cases, the at least one molecule comprises a dendrimer. In some cases, the dendrimer comprises at least one surface group. In some cases, the dendrimer comprises up to 4096 surface groups. In some cases, vesicles comprise at least one compartment for containing a dendrimer. Manipulating charge interactions enables a user to control dendrimer packing within vesicles and vesicle formation. (Chendan Li et al., Heirarchal polyion complex vesicles from PAMAM dendrimers, Journal of Colloid and Interface Science, vol. 606, part 1, 2022, pages 307-316, ISSN 0021-9797, https://doi.org/10.1016/j.jcis.2021.07.140). In some cases, the vesicle comprises a macrovesicle. In some cases, the vesicle comprises a microvesicle, an ectosome, u a nanoparticle, a liposome, an exosome. In some cases, the vesicle is coupled to a synthetic molecule disclosed herein. In some cases, the vesicle contains a synthetic molecule disclosed herein.

A fluorescent quencher as described herein may be in any structure. In some embodiments, the carrier may be a native, labeled or synthetic protein, a synthetic chemical polymer of precisely known chemical composition or with a distribution around a mean molecular weight (e.g. a linear or branched PEG polymers), an oligonucleotide, a phosphorodiamidate morpholino oligomer (PMO), or a foldamer, a lipid, a lipid micelle, a nanoparticle (e.g., iron oxide, gold, and non-metallic nanoparticles), a solid support made of polystyrene, polypropylene or any other type of plastic or polymer. In some embodiments, the carrier may be a peptide longer than a peptide linker. A carrier can be covalently or non-covalently attached to a linker or to a plurality of linkers.

In some embodiments, the carrier may be a nanoparticle. The transport of insoluble drugs via nanoparticles is improving because of their small particle size. Nanoparticle carrier is a kind of sub-micro particle delivery system, which belongs to a nanoscale microscope. Drugs encapsulated in sub-particles can adjust the speed of drug release, increase the permeability of biofilm, change the distribution in vivo, and improve the bioavailability. Nanoparticles are solid colloidal particles ranging in size from 10 to 100 nm used as a core in functionalization systems. They are generally composed of natural or synthetic macromolecule substances and can be used as carriers for conducting or transporting drugs. Nanospheres and nanocapsules can be formed. The chemical materials of nanomaterials are chitosan, gelatin, branched polymers, carbon-based carriers, etc. Gold nanoparticles consist of a core of gold atoms that can be functionalized by addition of a monolayer of moieties containing a thiol (SH) group.

In some embodiments, the carrier comprises a native, labeled or synthetic protein. Proteins can be used as carriers for the delivery of chemicals and biomolecular drugs, such as anticancer drugs and therapeutic proteins. Protein nanoparticles have several advantages as a drug delivery system, such as biodegradability, stability, surface modification of particles, ease of particle size control, and they have less problems associated with toxicity issues, such as immunogenicity. Protein nanoparticles can be generated using proteins, such as fibroins, albumin, gelatin, gliadin, legumin, 30Kc19, lipoprotein, and ferritin proteins, and are prepared through emulsion, electrospray, and desolvation methods. Hong S, Choi D W, Kim H N, Park C G, Lee W, Park H H. Protein-Based Nanoparticles as Drug Delivery Systems. Pharmaceutics. 2020; 12 (7): 604. Published 2020 Jun. 29. For example, albumin, a plasma protein with a molecular weight of 66 kDa, has been extensively investigated as a drug carrier.

In some embodiments, the carrier may be a synthetic chemical polymer. Polymeric nanoparticles have been extensively investigated as drug nanocarriers. Drug loading is achieved either by (i) entrapment of an aqueous drug phase using the polymer to form nanoscale structures such as cages and capsules or (ii) chemical linking of the drug molecules to the polymer backbone by means of a simple ester or amide bond that can be hydrolyzed in vivo. The most widely researched synthetic polymers include polylactide (PLA), poly(D,L-lactide-co-glycolide) (PLGA) and PEG. All three polymers are hydrolyzed in vivo and are biodegradable. Malam Y, Loizidou M, Seifalian A M. Liposomes and nanoparticles: nanosized vehicles for drug delivery in cancer. Trends Pharmacol Sci. 2009 November; 30 (11): 592-9.

In some embodiments, the carrier comprises a polyethylene glycol (PEG). PEG is used as a carrier because it is soluble in both organic and hydrophilic solvents. Unlike many other synthetic polymers, PEG is relatively hydrophilic. Conjugation with PEG increases the solubility of hydrophobic molecules and prolongs the circulation time in an organism. PEG also minimizes the nonspecific absorption of a molecule, such as a drug, provides specific affinity toward the targeted tissue, and increases the drug accumulation in malignant tissue. PEG can be conjugated to other polymers to make them less hydrophobic (i.e., PEGylation). The changes in surface hydrophilicity prevent protein adsorption, thereby enabling cell adhesion and proliferation on biomaterial scaffolds. The PMO backbone is made of morpholino rings with phosphorodiamidate linkage, which protects them from nuclease degradation while still maintaining the complementary base pairing. The potential application of PMO-based antisense technology targeting bacterial pathogens is being explored for the development of a new class of antibacterial drugs. Panchal R G, Geller B L, Mellbye B, Lane D, Iversen P L, Bavari S. Peptide conjugated phosphorodiamidate morpholino oligomers increase survival of mice challenged with Ames Bacillus anthracis. Nucleic Acid Ther. 2012; 22 (5): 316-322. Fluorescein-tagged Morpholinos combined with fluorescein-specific antibodies can be used as probes for in-situ hybridization to miRNAs.

In some embodiments, the carrier comprises an oligonucleotide. Biostable, high-payload DNA nanoassemblies of various structures, including cage-like DNA nanostructure, DNA particles, DNA polypods, and DNA hydrogel, have been reported. Cage-like DNA structures hold drug molecules firmly inside the structure and leave a large space within the cavity. These DNA nanostructures use their unique structure to carry abundant CpG, and their biocompatibility and size advantages to enter immune cells to achieve immunotherapy for various diseases. Part of the DNA nanostructures can also achieve more effective treatment in conjunction with other functional components such as aPD1, RNA, TLR ligands. DNA-based nanoparticles, such as spherical nucleic acids, hybrid DNA-based nanoparticles, polypod-like DNA nanostructure, DNA hydrogels have been reported. Chi Q, Yang Z, Xu K, Wang C and Liang H (2020) DNA Nanostructure as an Efficient Drug Delivery Platform for Immunotherapy. Front. Pharmacol. 10:1585.

In some embodiments, the carrier comprises a phosphorodiamidate Morpholino oligomer (PMO). Antisense phosphorodiamidate morpholino oligomers (PMOs) and their derivatives downregulate target gene expression in a sequence-dependent manner by interfering with the binding of ribosome to mRNA and thereby inhibiting protein translation.

In some embodiments, the carrier comprises a lipid or a lipid micelle. The liposome bilayer can be composed of either synthetic or natural phospholipids. The predominant physical and chemical properties of a liposome are based on the net properties of the constituent phospholipids, including permeability, charge density and steric hindrance. The lipid bilayer closes in on itself due to interactions between water molecules and the hydrophobic phosphate groups of the phospholipids. This process of liposome formation is spontaneous because the amphiphilic phospholipids self-associate into bilayers. Drug loading into liposomes can be achieved through (i) liposome formation in an aqueous solution saturated with soluble drug; (ii) the use of organic solvents and solvent exchange mechanisms; (iii) the use of lipophilic drugs; and (iv) pH gradient methods. Malam Y, Loizidou M, Seifalian A M. Liposomes and nanoparticles: nanosized vehicles for drug delivery in cancer. Trends Pharmacol Sci. 2009 November; 30 (11): 592-9.

In some embodiments, the carrier comprises a solid support made of polystyrene, polypropylene or any other type of plastic. For example, drug delivery properties of microporous polystyrene solid foams have been reported by Canal et al. These materials were obtained by polymerization in the continuous phase of highly concentrated emulsions prepared by the phase inversion temperature method. Their porosity, specific surface and surface topography are associated with drug incorporation and release characteristics. Canal, Cristina & Aparicio, Rosa & Vílchez, Alejandro & Esquena, Jordi & García-Celma, Maria. (2012). Drug Delivery Properties of Macroporous Polystyrene Solid Foams. Journal of pharmacy & pharmaceutical sciences: a publication of the Canadian Society for Pharmaceutical Sciences, Société canadienne des sciences pharmaceutiques. 15. 197-207.

In some embodiments, the carrier comprises a foldamer. In some embodiments, the foldamer includes a folded oligomer or polymer with a well-defined conformation. The conformation of foldamers is highly predictable from their primary sequences, therefore, it is possible to arrange functional groups at target positions and it may be possible to design functional foldamers, such as for efficient cellular uptake. For example, cell-penetrating peptide (CPP) foldamers are peptide-based foldamers equipped with cell membrane permeabilities. Peptide foldamers contain unnatural amino acids, non-proteinogenic amino acids, which make the peptide adopt a stable secondary structure, especially helical structures, even in short sequences. This property is helpful for the design of amphipathic CPPs with a stable helical structure. Furthermore, peptides containing unnatural amino acids generally exhibit resistance to hydrolysis by proteases, which are abundant throughout the body and in the cells. High stability of the peptide foldamers against enzymatic degradation can lead to their prolonged function in vivo. Makoto Oba, Cell-Penetrating Peptide Foldamers: Drug Delivery Tools. ChemBioChem 10.1002/cbic.201900204.

### Spacer

In some aspects, the synthetic molecule described herein comprises a spacer. In some aspects, the spacer is a self-immolative spacer. In some aspects, the self-immolative spacer comprise a disulfide, a p-amino benzyl alcohol, an a-quinone methide spacer, a hetheroaminebifuncional disulfide, a thiol-based pirydazinediones, a p-aminebenzyloxycarbonyl, a dipeptide, a Gly-Pro, a L-Phe-Sar, a trans-cyclooctene tetrazine, a ortho Hydroxy-protected Aryl sulfate, a phosphoramidate-based spacer, a hydroxybenzyl, a trimethyl carbamate, a quinone methide-based spacer, a cyclizing spacer, a Trimethyl lock, a 2-amino methyl piperidine or an ethylene diamine derived cyclizing spacer. Gonzaga et al. Perspective about self-immolative drug delivery systems. Journal of Pharmaceutical Sciences 109 (2020) 3262-3281.

Cleavage of the linker by a protease or enzyme present in an environment makes the self-immolative spacer dissociate from the precipitating fluorescent or non-fluorescent reporter, thereby resulting in a detectable signal. In some embodiments, the linker is cleavable by a predetermined endoprotease in the body fluid sample resulting in auto immolation and reporter release or results in a protease substrate that can be cleaved by a predetermined exopeptidase. In some embodiments, the predetermined exopeptidase is added to the body fluid sample. In some embodiments, the predetermined exopeptidase cleaves the protease substrate, thereby causing the self-immolative spacer to dissociate from the precipitating fluorescent reporter, thereby resulting in a detectable signal. In some aspects, the spacer is a component of the linker.

### Detection Via Synthetic Molecule

### Detecting Activity in an Environment

In an aspect, a method comprises introducing a synthetic molecule to an environment, wherein the synthetic molecule comprises a synthetic polymer, a linker, and a peptide sequence. The peptide sequence comprises a site, wherein the site is configurable to bind to an agent in an environment, a peptide spacer sequence, and a reporter. In some aspects, the method comprises release of a reporter coupled to the peptide sequence via an agent present in an environment reacting with the peptide sequence. Once the synthetic molecule is introduced into an environment, an agent present in the environment having an affinity to a site on the peptide sequence contacts the site. The agent contacts the site, triggering a reaction that releases the reporter in the environment. In some embodiments, the reaction comprises cleavage of the linker or a linker sequence. Upon release, the reporter generates a signal. The strength of the signal indicates a level of activity of the agent. For example, a stronger detected signal indicates a higher concentration of released reporters in an environment. In some aspects, the agent is an enzyme. In some aspects the enzyme is a protease. In some aspects, the environment comprises a subject. In some aspects, the subject comprises a human. In some aspects, the environment comprises a biofluid or a tissue. In some aspects, the biofluid comprises plasma, urine, saliva, feces, sputum, synovial fluid, cerebrospinal fluid, ascites fluid, or a combination thereof.

In some aspects, the method for detecting activity in a body fluid sample, as disclosed herein, comprises contacting the synthetic molecule to a body fluid sample. In some cases, the synthetic molecule is configured to detect an activity of one or more agents in the environment. In some cases, the one or more agents comprise an enzyme. In some cases, the enzyme comprises a protease. In some cases, the detected activity comprises a disease-related activity, a condition-related activity, a baseline activity, or a combination thereof. In some cases, the method comprises detecting a protease activity. In some cases, the protease activity comprises a baseline protease activity, a disease-related activity, a condition-related activity, or a combination thereof. The synthetic molecule disclosed herein is configured to detect enzyme activity with significantly higher specificity and sensitivity than linear peptides. In some exemplary cases, the synthetic molecule disclosed herein is configured to detect enzyme activity with approximately between 10× and 150× higher enzyme efficiency compared to a linear peptide subject to the same reaction conditions.

### Linker

In some aspects, the synthetic molecule described herein comprises a linker. The linker as described herein can be in any structure that is capable of being cleaved by an agent. In some embodiments, the linker comprises a peptide, a carbohydrate, a nucleic acid, a lipid, an ester, a glycoside, a phospholipid, a phosphodiester, a nucleophile/base sensitive linker, a reduction sensitive linker, an electrophile/acid sensitive linker, a metal linker, an oxidation sensitive linker, an auto-immolable linker (three component probe=enzyme substrate+linker+reporter) or a combination thereof. In some embodiments, the reporter can be in an inactive form and under disease activity becomes detectable. Geoffray Leriche, Louise Chisholm, Alain Wagner, Cleavable linkers in chemical biology, Bioorganic & Medicinal Chemistry, Volume 20, Issue 2, 2012, Pages 571-582, ISSN 0968-0896, https://doi.org/10.1016/j.bmc.2011.07.048.

Cross-linking agents aim to form a covalent bond between two spatially adjacent residues within one or two polymer chains. To identify protein binding partners, the cross-linking agents need to be able to detect and stabilize transient interactions. The crosslinking agents frequently form covalent links between lysine or cysteine residues in the proteins. Alternatively, the cross-linking agent can be photoreactive. Cross-linking linkers can be used to distinguish between inter- and intra-protein interactions of receptors, signaling cascades, and the structure of multi-protein complexes.

In some embodiments, the linker comprises a peptide. The core structure of a peptide linker can include either a di-peptide or a tetra-peptide that is recognized and cleaved by lysosomal enzymes. Proteases (also referred to as peptidases) catalyze the breakdown of peptide bonds by hydrolysis and is restricted to a specific sequence of amino acids recognizable by the proteases. Commonly used proteases comprise pepsin, trypsin or chymotrypsin. Since proteases have key roles in many diseases, peptide linkers are widely used in drug release systems or in diagnostic tools. In some embodiments, the peptide linkers comprise a short peptide sequence. In some embodiments, the peptide linkers comprise at least one non-naturally occurring amino acid.

In some embodiments, the peptide linkers can be less than about 20 amino acids in length. In some embodiments, the peptide linkers can be between 10 and 100 amino acids in length. In some embodiments, the peptide linkers can be 1 to 5, 1 to 10, 1 to 20, 1 to 30, 1 to 50, 1 to 70, 1 to 90, 1 to 100, 5 to 10, 5 to 20, 5 to 30, 5 to 50, 5 to 70, 5 to 90, 5 to 100, 10 to 20, 10 to 30, 10 to 50, 10 to 70, 10 to 90, 10 to 100, 20 to 30, 20 to 50, 20 to 70, 20 to 90, 20 to 100, 30 to 50, 30 to 70, 30 to 90, 30 to 100, 50 to 70, 50 to 90, 50 to 100, 70 to 90, 70 to 100, or 90 to 100 amino acids in length.

TABLE 1
Exemplary probe constructs.

| SEQ | | | | |
|---|---|---|---|---|
| ID | | Exemplary | | SEQ |
| NO | Sequence | probe name | Exemplary probe construct | ID NO |
| 1 | SGRSG | Probe #1 | 5-FAM-GSGRSGGK(CPQ2)-PEG2-kk-GC | 678 |
| 2 | PGPREG | Probe #2 | 5-FAM-GPGPREGGK(CPQ2)-PEG2-kk-GC | 679 |
| 3 | IEPDSGSQ | Probe #3 | 5-FAM-GIEPDSGSQGK(CPQ2)-PEG2-kk-GC | 680 |
| 4 | VVADSSMES | Probe #4 | 5-FAM-GVVADSSMESGK(CPQ2)-PEG2-kk- | 681 |
| | | | GC | |
| 5 | PTSY | Probe #5 | 5-FAM-GPTSYGK(CPQ2)-PEG2-kk-GC | 682 |
| 6 | YRFK | Probe #6 | 5-FAM-GYRFKGK(CPQ2)-PEG2-kk-GC | 683 |
| 7 | KVPL | Probe #7 | 5-FAM-GKVPLGK(CPQ2)-PEG2-kk-GC | 684 |
| 8 | VDVAD | Probe #8 | 5-FAM-GVDVADGK(CPQ2)-PEG2-kk-GC | 685 |
| 9 | LETD | Probe #9 | 5-FAM-GLETDGK(CPQ2)-PEG2-kk-GC | 686 |
| 10 | LEHD | Probe #10 | 5-FAM-GLEHDGK(CPQ2)-PEG2-kk-GC | 687 |
| 11 | REQD | Probe #11 | 5-FAM-GREQDGK(CPQ2)-PEG2-kk-GC | 688 |
| 12 | DEVD | Probe #12 | 5-FAM-GDEVDGK(CPQ2)-PEG2-kk-GC | 689 |
| 13 | VEID | Probe #13 | 5-FAM-GVEIDGK(CPQ2)-PEG2-kk-GC | 690 |
| 14 | VQVDGW | Probe #14 | 5-FAM-GVQVDGWGK(CPQ2)-PEG2-kk-GC | 691 |
| 15 | YEVDGW | Probe #15 | 5-FAM-GYEVDGWGK(CPQ2)-PEG2-kk-GC | 692 |
| 16 | LEVD | Probe #16 | 5-FAM-GLEVDGK(CPQ2)-PEG2-kk-GC | 693 |
| 17 | IEVE | Probe #17 | 5-FAM-GIEVEGK(CPQ2)-PEG2-kk-GC | 694 |
| 18 | AAPV | Probe #18 | 5-FAM-GAAPVGK(CPQ2)-PEG2-kk-GC | 695 |
| 19 | FFKF | Probe #19 | 5-FAM-GFFKFGK(CPQ2)-PEG2-kk-GC | 696 |
| 20 | GRRGKGG | Probe #20 | 5-FAM-GGRRGKGGGK(CPQ2)-PEG2-kk-GC | 697 |
| 21 | VKKR | Probe #21 | 5-FAM-GVKKRGK(CPQ2)-PEG2-kk-GC | 698 |
| 22 | FAAF(NO2)FV | Probe #22 | 5-FAM-GFAAF(NO2)FVL GK(CPQ2)-PEG2- | 699 |
| | L | | kk-GC | |
| 23 | VVR | Probe #23 | 5-FAM-GVVRGK(CPQ2)-PEG2-kk-GC | 700 |
| 24 | KQKLR | Probe #24 | 5-FAM-GKQKLRGK(CPQ2)-PEG2-kk-GC | 701 |
| 25 | RPPGFSAF | Probe #25 | 5-FAM-GRPPGFSAFGK(CPQ2)-PEG2-kk-GC | 702 |
| 26 | GPR | Probe #26 | 5-FAM-GGPRGK(CPQ2)-PEG2-kk-GC | 703 |
| 27 | FR | Probe #27 | 5-FAM-GFRGK(CPQ2)-PEG2-kk-GC | 704 |
| 28 | LPLGL | Probe #28 | 5-FAM-GLPLGLGK(CPQ2)-PEG2-kk-GC | 705 |
| 29 | KPLGL | Probe #29 | 5-FAM-GKPLGLGK(CPQ2)-PEG2-kk-GC | 706 |
| 30 | (Gaba)PQGLE | Probe #30 | 5-FAM-G(Gaba)PQGLE GK(CPQ2)-PEG2-kk- | 707 |
| | | | GC | |
| 31 | PKPLAL | Probe #31 | 5-FAM-GPKPLALGK(CPQ2)-PEG2-kk-GC | 708 |
| 32 | GPSGIHV | Probe #32 | 5-FAM-GGPSGIHVGK(CPQ2)-PEG2-kk-GC | 709 |
| 33 | WAHRTTFYR | Probe #33 | 5-FAM-GWAHRTTFYRRGAGK(CPQ2)- | 710 |
| | RGA | | PEG2-kk-GC | |
| 34 | WKLRSSKQ | Probe #34 | 5-FAM-GWKLRSSKQGK(CPQ2)-PEG2-kk- | 711 |
| | | | GC | |
| 35 | PFR | Probe #35 | 5-FAM-GPFRGK(CPQ2)-PEG2-kk-GC | 712 |
| 36 | SYRIF | Probe #36 | 5-FAM-GSYRIFGK(CPQ2)-PEG2-kk-GC | 713 |
| 37 | RPY | Probe #37 | 5-FAM-GRPYGK(CPQ2)-PEG2-kk-GC | 714 |
| 38 | TAFRSAYG | Probe #38 | 5-FAM-GTAFRSAYGGK(CPQ2)-PEG2-kk-GC | 715 |
| 39 | WAAFRFSQA | Probe #39 | 5-FAM-GWAAFRFSQAGK(CPQ2)-PEG2-kk- | 716 |
| | | | GC | |
| 40 | VPR | Probe #40 | 5-FAM-GVPRGK(CPQ2)-PEG2-kk-GC | 717 |
| 41 | G | Probe #41 | 5-FAM-GGK(CPQ2)-PEG2-kk-GC | 718 |
| 42 | KLRSSKQ | Probe #42 | 5-FAM-GKLRSSKQGK(CPQ2)-PEG2-kk-GC | 719 |
| 43 | YASR | Probe #43 | 5-FAM-GYASRGK(CPQ2)-PEG2-kk-GC | 720 |
| 44 | RFAQAQQQL | Probe #44 | 5-FAM-GRFAQAQQQLPGK(CPQ2)-PEG2-kk- | 721 |
| | P | | GC | |
| 45 | KPAKFFRL | Probe #45 | 5-FAM-GKPAKFFRLGK(CPQ2)-PEG2-kk-GC | 722 |
| 46 | PRAAA(hF)TS | Probe #46 | 5-FAM-GPRAAA(hF)TSPGK(CPQ2)-PEG2- | 723 |
| | P | | kk-GC | |
| 47 | VGPQRFSGAP | Probe #47 | 5-FAM-GVGPQRFSGAPGK(CPQ2)-PEG2-kk- | 724 |
| | | | GC | |
| 48 | FFLAQA(hF)R | Probe #48 | 5-FAM-GFFLAQA(hF)RS GK(CPQ2)-PEG2- | 725 |
| | S | | kk-GC | |
| 49 | PLAQAV | Probe #49 | 5-FAM-GPLAQAVGK(CPQ2)-PEG2-kk-GC | 726 |
| 50 | RTAAVFRP | Probe #50 | 5-FAM-GRTAAVFRPGK(CPQ2)-PEG2-kk-GC | 727 |
| 51 | DVQEFRGVT | Probe #51 | 5-FAM-GDVQEFRGVTAVIRGK(CPQ2)- | 728 |
| | AVIR | | PEG2-kk-GC | |
| 52 | TEGEARGSVI | Probe #52 | 5-FAM-GTEGEARGSVIGK(CPQ2)-PEG2-kk- | 729 |
| | | | GC | |
| 53 | 1-TR | Probe #53 | 5-FAM-G-1-TRGK(CPQ2)-PEG2-kk-GC | 730 |
| 54 | PLFAERK | Probe #54 | 5-FAM-GPLFAERKGK(CPQ2)-PEG2-kk-GC | 731 |
| 55 | LLVY | Probe #55 | 5-FAM-GLLVYGK(CPQ2)-PEG2-kk-GC | 732 |
| 56 | QQKRKIVL | Probe #56 | 5-FAM-GQQKRKIVLGK(CPQ2)-PEG2-kk-GC | 733 |
| 57 | ASHLGLAR | Probe #57 | 5-FAM-GASHLGLARGK(CPQ2)-PEG2-kk-GC | 734 |
| 58 | LPSRSSKI | Probe #58 | 5-FAM-GLPSRSSKIGK(CPQ2)-PEG2-kk-GC | 735 |
| 59 | STGRNGFK | Probe #59 | 5-FAM-GSTGRNGFKGK(CPQ2)-PEG2-kk-GC | 736 |
| 60 | SLLRSEET | Probe #60 | 5-FAM-GSLLRSEETGK(CPQ2)-PEG2-kk-GC | 737 |
| 61 | HRGRTLEI | Probe #61 | 5-FAM-GHRGRTLEIGK(CPQ2)-PEG2-kk-GC | 738 |
| 62 | YLGRSYKV | Probe #62 | 5-FAM-GYLGRSYKVGK(CPQ2)-PEG2-kk- | 739 |
| | | | GC | |
| 63 | EKQRIIGG | Probe #63 | 5-FAM-GEKQRIIGGGK(CPQ2)-PEG2-kk-GC | 740 |
| 64 | QRQRIIGG | Probe #64 | 5-FAM-GQRQRIIGGGK(CPQ2)-PEG2-kk-GC | 741 |
| 65 | LORIYK | Probe #65 | 5-FAM-GLQRIYKGK(CPQ2)-PEG2-kk-GC | 742 |
| 66 | SLGRKIQI | Probe #66 | 5-FAM-GSLGRKIQIGK(CPQ2)-PEG2-kk-GC | 743 |
| 67 | HAAPRSADIQ | Probe #67 | 5-FAM-GHAAPRSADIQIDIGK(CPQ2)-PEG2- | 744 |
| | IDI | | kk-GC | |
| 68 | FGR | Probe #68 | 5-FAM-GFGRGK(CPQ2)-PEG2-kk-GC | 745 |
| 69 | SLGR | Probe #69 | 5-FAM-GSLGRGK(CPQ2)-PEG2-kk-GC | 746 |
| 70 | GLQR | Probe #70 | 5-FAM-GGLQRGK(CPQ2)-PEG2-kk-GC | 747 |
| 71 | SVARTLLV | Probe #71 | 5-FAM-GSVARTLLVGK(CPQ2)-PEG2-kk-GC | 748 |
| 72 | GRIFG | Probe #72 | 5-FAM-GGRIFGGK(CPQ2)-PEG2-kk-GC | 749 |
| 73 | APK | Probe #73 | 5-FAM-GAPKGK(CPQ2)-PEG2-kk-GC | 750 |
| 74 | GFSPY | Probe #74 | 5-FAM-GGFSPYGK(CPQ2)-PEG2-kk-GC | 751 |
| 75 | WELRHAGH | Probe #75 | 5-FAM-GWELRHAGHGK(CPQ2)-PEG2-kk- | 752 |
| | | | GC | |
| 76 | RQSRIVGGE | Probe #76 | 5-FAM-GRQSRIVGGEGK(CPQ2)-PEG2-kk- | 753 |
| | | | GC | |
| 77 | EQAVYQTI | Probe #77 | 5-FAM-GEQAVYQTIGK(CPQ2)-PEG2-kk-GC | 754 |
| 78 | VAYSGENTF | Probe #78 | 5-FAM-GVAYSGENTFGFGK(CPQ2)-PEG2- | 755 |
| | GF | | kk-GC | |
| 79 | GGR | Probe #79 | 5-FAM-GGGRGK(CPQ2)-PEG2-kk-GC | 756 |
| 80 | ATAD | Probe #80 | 5-FAM-GATADGK(CPQ2)-PEG2-kk-GC | 757 |
| 81 | RPLESNAV | Probe #81 | 5-FAM-GRPLESNAVGK(CPQ2)-PEG2-kk-GC | 758 |
| 82 | RPLGLAR | Probe #82 | 5-FAM-GRPLGLARGK(CPQ2)-PEG2-kk-GC | 759 |
| 83 | AAFF | Probe #83 | 5-FAM-GAAFFGK(CPQ2)-PEG2-kk-GC | 760 |
| 84 | RVKRGLA | Probe #84 | 5-FAM-GRVKRGLAGK(CPQ2)-PEG2-kk-GC | 761 |
| 85 | AAL | Probe #85 | 5-FAM-GAALGK(CPQ2)-PEG2-kk-GC | 762 |
| 86 | CGGmeGVndn | Probe #86 | 5-FAM-CGGmeGVndneeGFFsArGK(CPQ2) | 763 |
| | eeGFFsAr | | | |
| 87 | GPQGIWGQ | Probe #87 | 5FAM-GGPQGIWGQK(CPQ2)-PEG2-C | 764 |
| 88 | GLVPRGS | Probe #88 | 5FAM-GGLVPRGSGK(CPQ2)-PEG2-C | 765 |
| 89 | GPVGLI | Probe #89 | 5FAM-GGPVGLIGK(CPQ2)-PEG2-C | 766 |
| 90 | GPWGIWGQ | Probe #90 | 5FAM-GGPWGIWGQGK(CPQ2)-PEG2-C | 767 |
| 91 | GPVPLSLVM | Probe #91 | 5FAM-GGPVPLSLVMK(CPQ2)-PEG2-C | 768 |
| 92 | Gf-Pip-RSGG | Probe #92 | 5FAM-GGf-Pip-RSGGGK(CPQ2)-PEG2-C | 769 |
| 93 | PLGMRG | Probe #93 | 5FAM-GGf-Pip-KSGGGK(CPQ2)-PEG2-C | 770 |
| 94 | PLGMRG | Probe #94 | (FAM)-GPLGMRGG-K(CPQ2)-PEG2-k-GC | 771 |
| 95 | P-(Cha)-G- | Probe #95 | (FAM)-GP-(Cha)-G-Cys(Me)-HAG-K(CPQ2)- | 772 |
| | Cys(Me)-HA | | PEG2-kk-GC | |
| 96 | RPLALWESQ | Probe #96 | (FAM)-GRPLALWESQG-K(CPQ2)-PEG2-k- | 773 |
| | | | GC | |
| 97 | SGKGPRQITA | Probe #97 | (FAM)-SGKGPRQITA-K(CPQ2)-PEG2-k-GC | 774 |
| 98 | SGPLFYSVTA | Probe #98 | (FAM)-SGPLFYSVTA-K(CPQ2)-PEG2-kk-GC | 775 |
| 99 | SGRIFLRTA | Probe #99 | (FAM)-SGRIFLRTA-K(CPQ2)-PEG2-GC | 776 |
| 100 | SGRSENIRTA | Probe #100 | (FAM)-SGRSENIRTA-K(CPQ2)-PEG2-GC | 777 |
| 101 | GSGGS | Probe #101 | (FAM)-GGSGGS-K(CPQ2)-PEG2-kk-GC | 778 |
| 102 | KPILFFRLKG | Probe #102 | (FAM)-GKPILFFRLKG-K(CPQ2)-PEG2-kk- | 779 |
| | | | GC | |
| 103 | AWESR(Nle) | Probe #103 | (FAM)-GAWESR(Nle)GK(CPQ2)-NH2 | 780 |
| 104 | NEKSG(Nle) | Probe #104 | (FAM)-GNEKSG(Nle)GK(CPQ2)-NH2 | 781 |
| 105 | NATIVY | Probe #105 | (FAM)-GNATIVYGK(CPQ2)-PEG2-k-NH2 | 782 |
| 106 | DPFVVS | Probe #106 | (FAM)-GDPFVVSGK(CPQ2)-PEG2-k-NH2 | 783 |
| 107 | FH(Nle)FTK | Probe #107 | (FAM)-GFH(Nle)FTKGK(CPQ2)-PEG2-k-NH2 | 784 |
| 108 | (Nle)NWHKH | Probe #108 | (FAM)-G(Nle)NWHKHGK(CPQ2)-NH2 | 785 |
| 109 | FARRWG | Probe #109 | (FAM)-GFARRWGGK(CPQ2)-PEG2-k-NH2 | 786 |
| 110 | PGKWSK | Probe #110 | (FAM)-GPGKWSKGK(CPQ2)-PEG2-k-NH2 | 787 |
| 111 | YEEAQP | Probe #111 | (FAM)-GYEEAQPGK(CPQ2)-PEG2-k-NH2 | 788 |
| 112 | YGAIKK | Probe #112 | (FAM)-GYGAIKKGK(CPQ2)-PEG2-k-NH2 | 789 |
| 113 | TS(Nle)EGY | Probe #113 | (FAM)-GTS(Nle)EGYGK(CPQ2)-PEG2-k | 790 |
| 114 | PNNFGS | Probe #114 | (FAM)-GPNNFGSGK(CPQ2)-PEG2-k-NH2 | 791 |
| 115 | EDTRNT | Probe #115 | (FAM)-GEDTRNTGK(CPQ2)-NH2 | 792 |
| 116 | KDLEQS | Probe #116 | (FAM)-GKDLEQSGK(CPQ2)-NH2 | 793 |
| 117 | AALHND | Probe #117 | (FAM)-GAALHNDGK(CPQ2)-PEG2-kk-NH2 | 794 |
| 118 | ADSFFK | Probe #118 | (FAM)-GADSFFKGK(CPQ2)-NH2 | 795 |
| 119 | ITFWRA | Probe #119 | (FAM)-GITFWRAGK(CPQ2)-NH2 | 796 |
| 120 | LSD(Nle)RL | Probe #120 | (FAM)-GLSD(Nle)RLGK(CPQ2)-NH2 | 797 |
| 121 | EVGWTY | Probe #121 | (FAM)-GEVGWTYGK(CPQ2)-PEG2-k-NH2 | 798 |
| 122 | IAFRQ(Nle) | Probe #122 | (FAM)-GIAFRQ(Nle)GK(CPQ2)-NH2 | 799 |
| 123 | YNIHT(Nle) | Probe #123 | (FAM)-GYNIHT(Nle)GK(CPQ2)-PEG2-kk- | 800 |
| | | | NH2 | |
| 124 | (Nle)LWANH | Probe #124 | (FAM)-G(Nle)LWANHGK(CPQ2)-PEG2-kk- | 801 |
| | | | NH2 | |
| 125 | LYSVQV | Probe #125 | (FAM)-GLYSVQVGK(CPQ2)-PEG2-k-NH2 | 802 |
| 126 | SHI(Nle)SN | Probe #126 | (FAM)-GSHI(Nle)SNGK(CPQ2)-PEG2-kk- | 803 |
| | | | NH2 | |
| 127 | KLLIDV | Probe #127 | (FAM)-GKLLIDVGK(CPQ2)-NH2 | 804 |
| 128 | E(Nle)GVFD | Probe #128 | (FAM)-GE(Nle)GVFDGK(CPQ2)-PEG2-k-NH2 | 805 |
| 129 | HQAYTL | Probe #129 | (FAM)-GHQAYTLGK(CPQ2)-PEG2-kk-NH2 | 806 |
| 130 | YVRKIQ | Probe #130 | (FAM)-GYVRKIQGK(CPQ2)-PEG2-k-NH2 | 807 |
| 131 | DRENSP | Probe #131 | (FAM)-GDRENSPGK(CPQ2)-NH2 | 808 |
| 132 | KYDKPR | Probe #132 | (FAM)-GKYDKPRGK(CPQ2)-NH2 | 809 |
| 133 | RPWKQL | Probe #133 | (FAM)-GRPWKQLGK(CPQ2)-PEG2-k-NH2 | 810 |
| 134 | APLQRY | Probe #134 | (FAM)-GAPLQRYGK(CPQ2)-NH2 | 811 |
| 135 | YQGQK(Nle) | Probe #135 | (FAM)-GYQGQK(Nle)GK(CPQ2)-NH2 | 812 |
| 136 | GRISSI | Probe #136 | (FAM)-GGRISSIGK(CPQ2)-NH2 | 813 |
| 137 | HSLTNV | Probe #137 | (FAM)-GHSLTNVGK(CPQ2)-PEG2-kk-NH2 | 814 |
| 138 | EWDFPE | Probe #138 | (FAM)-GEWDFPEGK(CPQ2)-PEG2-k-NH2 | 815 |
| 139 | YLA(Nle)DG | Probe #139 | (FAM)-GYLA(Nle)DGGK(CPQ2)-PEG2-k- | 816 |
| | | | NH2 | |
| 140 | FIY(Nle)PT | Probe #140 | (FAM)-GFIY(Nle)PTGK(CPQ2)-PEG2-k-NH2 | 817 |
| 141 | GHETWV | Probe #141 | (FAM)-GGHETWVGK(CPQ2)-PEG2-kk-NH2 | 818 |
| 142 | DYIGDE | Probe #142 | (FAM)-GDYIGDEGK(CPQ2)-PEG2-k-NH2 | 819 |
| 143 | AGTAHP | Probe #143 | (FAM)-GAGTAHPGK(CPQ2)-PEG2-kk-NH2 | 820 |
| 144 | V(Nle)TEIW | Probe #144 | (FAM)-GV(Nle)TEIWGK(CPQ2)-PEG2-k-NH2 | 821 |
| 145 | PDDWQN | Probe #145 | (FAM)-GPDDWQNGK(CPQ2)-PEG2-k-NH2 | 822 |
| 146 | GLNQEY | Probe #146 | (FAM)-GGLNQEYGK(CPQ2)-PEG2-k-NH2 | 823 |
| 147 | YRDAVA | Probe #147 | (FAM)-GYRDAVAGK(CPQ2)-NH2 | 824 |
| 148 | TGPKGN | Probe #148 | (FAM)-GTGPKGNGK(CPQ2)-NH2 | 825 |
| 149 | DHVPQI | Probe #149 | (FAM)-GDHVPQIGK(CPQ2)-PEG2-kk-NH2 | 826 |
| 150 | NKEPIL | Probe #150 | (FAM)-GNKEPILGK(CPQ2)-NH2 | 827 |
| 151 | VWN(Nle)VH | Probe #151 | (FAM)-GVWN(Nle)VHGK(CPQ2)-PEG2-kk- | 828 |
| | | | NH2 | |
| 152 | PVIIEH | Probe #152 | (FAM)-GPVIIEHGK(CPQ2)-PEG2-kk-NH2 | 829 |
| 153 | FQTDNL | Probe #153 | (FAM)-GFQTDNLGK(CPQ2)-PEG2-k-NH2 | 830 |
| 154 | RF(Nle)HGI | Probe #154 | (FAM)-GRF(Nle)HGIGK(CPQ2)-PEG2-k-NH2 | 831 |
| 155 | YAERTT | Probe #155 | (FAM)-GYAERTTGK(CPQ2)-NH2 | 832 |
| 156 | NRGELP | Probe #156 | (FAM)-GNRGELPGK(CPQ2)-NH2 | 833 |
| 157 | HHYFNY | Probe #157 | (FAM)-GHHYFNYGK(CPQ2)-PEG2-k-NH2 | 834 |
| 158 | STPYYH | Probe #158 | (FAM)-GSTPYYHGK(CPQ2)-PEG2-kk-NH2 | 835 |
| 159 | WFYPSA | Probe #159 | (FAM)-GWFYPSAGK(CPQ2)-PEG2-k-NH2 | 836 |
| 160 | SEFLFS | Probe #160 | (FAM)-GSEFLFSGK(CPQ2)-PEG2-k-NH2 | 837 |
| 161 | WYKTQY | Probe #161 | (FAM)-GWYKTQYGK(CPQ2)-NH2 | 838 |
| 162 | VTHLKV | Probe #162 | (FAM)-GVTHLKVGK(CPQ2)-PEG2-k-NH2 | 839 |
| 163 | INGGFS | Probe #163 | (FAM)-GINGGFSGK(CPQ2)-PEG2-k-NH2 | 840 |
| 164 | TVLGLD | Probe #164 | (FAM)-GTVLGLDGK(CPQ2)-PEG2-k-NH2 | 841 |
| 165 | SYWP(Nle)Q | Probe #165 | (FAM)-GSYWP(Nle)QGK(CPQ2)-PEG2-k- | 842 |
| | | | NH2 | |
| 166 | ASQQHR | Probe #166 | (FAM)-GASQQHRGK(CPQ2)-PEG2-k-NH2 | 843 |
| 167 | KNPAKA | Probe #167 | (FAM)-GKNPAKAGK(CPQ2)-PEG2-k-NH2 | 844 |
| 168 | (Nle)YWLVE | Probe #168 | (FAM)-G(Nle)YWLVEGK(CPQ2)-PEG2-k- | 845 |
| | | | NH2 | |
| 169 | SWWIFE | Probe #169 | (FAM)-GSWWIFEGK(CPQ2)-PEG2-k-NH2 | 846 |
| 170 | VNYEQD | Probe #170 | (FAM)-GVNYEQDGK(CPQ2)-PEG2-k-NH2 | 847 |
| 171 | HFF(Nle)AE | Probe #171 | (FAM)-GHFF(Nle)AEGK(CPQ2)-PEG2-kk- | 848 |
| | | | NH2 | |
| 172 | DIPPHW | Probe #172 | (FAM)-GDIPPHWGK(CPQ2)-PEG2-kk-NH2 | 849 |
| 173 | VDQW(Nle)W | Probe #173 | (FAM)-GVDQW(Nle)WGK(CPQ2)-PEG2-k- | 850 |
| | | | NH2 | |
| 174 | LRSL(Nle)K | Probe #174 | (FAM)-GLRSL(Nle)KGK(CPQ2)-PEG2-k-NH2 | 851 |
| 175 | (Nle)(Nle)IRH | Probe #175 | (FAM)-G(Nle)(Nle)IRHAGK(CPQ2)-PEG2-k- | 852 |
| | A | | NH2 | |
| 176 | HDVKFI | Probe #176 | (FAM)-GHDVKFIGK(CPQ2)-PEG2-kk-NH2 | 853 |
| 177 | KRVQFL | Probe #177 | (FAM)-GKRVQFLGK(CPQ2)-PEG2-k-NH2 | 854 |
| 178 | RD(Nle)YAE | Probe #178 | (FAM)-GRD(Nle)YAEGK(CPQ2)-NH2 | 855 |
| 179 | L(Nle)IYFE | Probe #179 | (FAM)-GL(Nle)IYFEGK(CPQ2)-PEG2-k-NH2 | 856 |
| 180 | LRTKQS | Probe #180 | (FAM)-GLRTKQSGK(CPQ2)-PEG2-k-NH2 | 857 |
| 181 | WHGQQY | Probe #181 | (FAM)-GWHGQQYGK(CPQ2)-PEG2-kk-NH2 | 858 |
| 182 | GPEGTI | Probe #182 | (FAM)-GGPEGTIGK(CPQ2)-PEG2-k-NH2 | 859 |
| 183 | ELDPIP | Probe #183 | (FAM)-GELDPIPGK(CPQ2)-PEG2-k-NH2 | 860 |
| 184 | GRAADF | Probe #184 | (FAM)-GGRAADFGK(CPQ2)-NH2 | 861 |
| 185 | HFIDYI | Probe #185 | (FAM)-GHFIDYIGK(CPQ2)-PEG2-kk-NH2 | 862 |
| 186 | S(Nle)(Nle)RV | Probe #186 | (FAM)-GS(Nle)(Nle)RVHGK(CPQ2)-PEG2-k- | 863 |
| | H | | NH2 | |
| 187 | SFRKII | Probe #187 | (FAM)-GSFRKIIGK(CPQ2)-PEG2-k-NH2 | 864 |
| 188 | TYE(Nle)FS | Probe #188 | (FAM)-GTYE(Nle)FSGK(CPQ2)-PEG2-k-NH2 | 865 |
| 189 | HLLGFY | Probe #189 | (FAM)-GHLLGFYGK(CPQ2)-PEG2-kk-NH2 | 866 |
| 190 | (Nle)WTALT | Probe #190 | (FAM)-G(Nle)WTALTGK(CPQ2)-PEG2-k- | 867 |
| | | | NH2 | |
| 191 | IWN(Nle)VY | Probe #191 | (FAM)-GIWN(Nle)VYGK(CPQ2)-PEG2-k- | 868 |
| | | | NH2 | |
| 192 | RRNPLW | Probe #192 | (FAM)-GRRNPLWGK(CPQ2)-PEG2-k-NH2 | 869 |
| 193 | RWYGGI | Probe #193 | (FAM)-GRWYGGIGK(CPQ2)-NH2 | 870 |
| 194 | KTGDAR | Probe #194 | (FAM)-GKTGDARGK(CPQ2)-PEG2-k-NH2 | 871 |
| 195 | NYWEAN | Probe #195 | (FAM)-GNYWEANGK(CPQ2)-PEG2-k-NH2 | 872 |
| 196 | (Nle)QFDTS | Probe #196 | (FAM)-G(Nle)QFDTSGK(CPQ2)-PEG2-k-NH2 | 873 |
| 197 | KRGAVE | Probe #197 | (FAM)-GKRGAVEGK(CPQ2)-PEG2-k-NH2 | 874 |
| 198 | SLKPTE | Probe #198 | (FAM)-GSLKPTEGK(CPQ2)-NH2 | 875 |
| 199 | ENDRLP | Probe #199 | (FAM)-GENDRLPGK(CPQ2)-NH2 | 876 |
| 200 | NSYQVQ | Probe #200 | (FAM)-GNSYQVQGK(CPQ2)-PEG2-k-NH2 | 877 |
| 201 | YPKEYL | Probe #201 | (FAM)-GYPKEYLGK(CPQ2)-NH2 | 878 |
| 202 | INNKWQ | Probe #202 | (FAM)-GINNKWQGK(CPQ2)-NH2 | 879 |
| 203 | (Nle)EFQGW | Probe #203 | (FAM)-G(Nle)EFQGWGK(CPQ2)-PEG2-k- | 880 |
| | | | NH2 | |
| 204 | PVRSTN | Probe #204 | (FAM)-GPVRSTNGK(CPQ2)-NH2 | 881 |
| 205 | SQAIKV | Probe #205 | (FAM)-GSQAIKVGK(CPQ2)-NH2 | 882 |
| 206 | WA(Nle)LYH | Probe #206 | (FAM)-GWA(Nle)LYHGK(CPQ2)-PEG2-kk- | 883 |
| | | | NH2 | |
| 207 | ISWIHA | Probe #207 | (FAM)-GISWIHAGK(CPQ2)-PEG2-kk-NH2 | 884 |
| 208 | AHDIV | Probe #208 | (FAM)-GAHDIVNGK(CPQ2)-PEG2-kk-NH2 | 885 |
| 209 | RHNVAS | Probe #209 | (FAM)-GRHNVASGK(CPQ2)-PEG2-k-NH2 | 886 |
| 210 | SVFVIE | Probe #210 | (FAM)-GSVFVIEGK(CPQ2)-PEG2-k-NH2 | 887 |
| 211 | FAKYYK | Probe #211 | (FAM)-GFAKYYKGK(CPQ2)-PEG2-k-NH2 | 888 |
| 212 | PYNTLQ | Probe #212 | (FAM)-GPYNTLQGK(CPQ2)-PEG2-k-NH2 | 889 |
| 213 | (Nle)DWGH | Probe #213 | (FAM)-G(Nle)DWGH(Nle)GK(CPQ2)-PEG2- | 890 |
| | (Nle) | | kk-NH2 | |
| 214 | SNREWF | Probe #214 | (FAM)-GSNREWFGK(CPQ2)-NH2 | 891 |
| 215 | GKSEHT | Probe #215 | (FAM)-GGKSEHTGK(CPQ2)-PEG2-kk-NH2 | 892 |
| 216 | FP(Nle)TDQ | Probe #216 | (FAM)-GFP(Nle)TDQGK(CPQ2)-PEG2-k-NH2 | 893 |
| 217 | WSKFW(Nle) | Probe #217 | (FAM)-GWSKFW(Nle)GK(CPQ2) | 894 |
| 218 | RFTRPH | Probe #218 | (FAM)-GRFTRPHGK(CPQ2)-NH2 | 895 |
| 219 | QET(Nle)KD | Probe #219 | (FAM)-GQET(Nle)KDGK(CPQ2)-NH2 | 896 |
| 220 | HWWDVL | Probe #220 | (FAM)-GHWWDVLGK(CPQ2)-PEG2-kk-NH2 | 897 |
| 221 | FNLV(Nle)S | Probe #221 | (FAM)-GFNLV(Nle)SGK(CPQ2)-PEG2-k-NH2 | 898 |
| 222 | SAWRQR | Probe #222 | (FAM)-GSAWRQRGK(CPQ2)-PEG2-k-NH2 | 899 |
| 223 | TFHIFL | Probe #223 | (FAM)-GTFHIFLGK(CPQ2)-PEG2-kk-NH2 | 900 |
| 224 | WPQHVK | Probe #224 | (FAM)-GWPQHVKGK(CPQ2)-PEG2-k-NH2 | 901 |
| 225 | LI(Nle)HKN | Probe #225 | (FAM)-GLI(Nle)HKNGK(CPQ2)-PEG2-k-NH2 | 902 |
| 226 | QDLEQP | Probe #226 | (FAM)-GQDLEQPGK(CPQ2)-PEG2-k-NH2 | 903 |
| 227 | HQKK(Nle)P | Probe #227 | (FAM)-GHQKK(Nle)PGK(CPQ2)-NH2 | 904 |
| 228 | GVTWLN | Probe #228 | (FAM)-GGVTWLNGK(CPQ2)-PEG2-k-NH2 | 905 |
| 229 | AGEPFK | Probe #229 | (FAM)-GAGEPFKGK(CPQ2)-NH2 | 906 |
| 230 | SR(Nle)ATT | Probe #230 | (FAM)-GSR(Nle)ATTGK(CPQ2)-NH2 | 907 |
| 231 | LAF(Nle)NH | Probe #231 | (FAM)-GLAF(Nle)NHGK(CPQ2)-PEG2-kk- | 908 |
| | | | NH2 | |
| 232 | PPSGLS | Probe #232 | (FAM)-GPPSGLSGK(CPQ2)-PEG2-k-NH2 | 909 |
| 233 | YTHSSP | Probe #233 | (FAM)-GYTHSSPGK(CPQ2)-PEG2-kk-NH2 | 910 |
| 234 | DGSHYR | Probe #234 | (FAM)-GDGSHYRGK(CPQ2)-PEG2-kk-NH2 | 911 |
| 235 | Y(Nle)GNGY | Probe #235 | (FAM)-GY(Nle)GNGYGK(CPQ2)-PEG2-k- | 912 |
| | | | NH2 | |
| 236 | DSITVS | Probe #236 | (FAM)-GDSITVSGK(CPQ2)-PEG2-k-NH2 | 913 |
| 237 | QTPNIQ | Probe #237 | (FAM)-GQTPNIQGK(CPQ2)-PEG2-k-NH2 | 914 |
| 238 | KLFFGY | Probe #238 | (FAM)-GKLFFGYGK(CPQ2)-NH2 | 915 |
| 239 | TQNFNW | Probe #239 | (FAM)-GTQNFNWGK(CPQ2)-PEG2-k-NH2 | 916 |
| 240 | YSDHEV | Probe #240 | (FAM)-GYSDHEVGK(CPQ2)-PEG2-kk-NH2 | 917 |
| 241 | RYVVPA | Probe #241 | (FAM)-GRYVVPAGK(CPQ2)-NH2 | 918 |
| 242 | ILHRIR | Probe #242 | (FAM)-GILHRIRGK(CPQ2)-NH2 | 919 |
| 243 | ESDNQ(Nle) | Probe #243 | (FAM)-GESDNQ(Nle)GK(CPQ2)-PEG2-k-NH2 | 920 |
| 244 | YDDKG(Nle) | Probe #244 | (FAM)-GYDDKG(Nle)GK(CPQ2)-NH2 | 921 |
| 245 | QLS(Nle)VW | Probe #245 | (FAM)-GQLS(Nle)VWGK(CPQ2)-PEG2-k- | 922 |
| | | | NH2 | |
| 246 | PGGER(Nle) | Probe #246 | (FAM)-GPGGER(Nle)GK(CPQ2)-NH2 | 923 |
| 247 | WKHHPD | Probe #247 | (FAM)-GWKHHPDGK(CPQ2)-NH2 | 924 |
| 248 | QWVDED | Probe #248 | (FAM)-GQWVDEDGK(CPQ2)-PEG2-k-NH2 | 925 |
| 249 | NAYNEI | Probe #249 | (FAM)-GNAYNEIGK(CPQ2)-PEG2-k-NH2 | 926 |
| 250 | EEKAPR | Probe #250 | (FAM)-GEEKAPRGK(CPQ2)-PEG2-kk-NH2 | 927 |
| 251 | PWQIGK | Probe #251 | (FAM)-GPWQIGKGK(CPQ2)-NH2 | 928 |
| 252 | IAQVGN | Probe #252 | (FAM)-GIAQVGNGK(CPQ2)-PEG2-k-NH2 | 929 |
| 253 | V(Nle)RQSE | Probe #253 | (FAM)-GV(Nle)RQSEGK(CPQ2)-NH2 | 930 |
| 254 | TERVDA | Probe #254 | (FAM)-GTERVDAGK(CPQ2)-NH2 | 931 |
| 255 | WLRWRL | Probe #255 | (FAM)-GWLRWRLGK(CPQ2)-PEG2-k-NH2 | 932 |
| 256 | WKTKGQ | Probe #256 | (FAM)-GWKTKGQGK(CPQ2)-PEG2-k-NH2 | 933 |
| 257 | QSNGDV | Probe #257 | (FAM)-GQSNGDVGK(CPQ2)-PEG2-k-NH2 | 934 |
| 258 | TLFYAL | Probe #258 | (FAM)-GTLFYALGK(CPQ2)-PEG2-k-NH2 | 935 |
| 259 | TVTLNP | Probe #259 | (FAM)-GTVTLNPGK(CPQ2)-PEG2-k-NH2 | 936 |
| 260 | YAFGRK | Probe #260 | (FAM)-GYAFGRKGK(CPQ2)-PEG2-k-NH2 | 937 |
| 261 | DYNYWD | Probe #261 | (FAM)-GDYNYWDGK(CPQ2)-PEG2-k-NH2 | 938 |
| 262 | EWHEII | Probe #262 | (FAM)-GEWHEIIGK(CPQ2)-PEG2-kk-NH2 | 939 |
| 263 | QKAAWD | Probe #263 | (FAM)-GQKAAWDGK(CPQ2)-NH2 | 940 |
| 264 | DNTSAD | Probe #264 | (FAM)-GDNTSADGK(CPQ2)-PEG2-k-NH2 | 941 |
| 265 | HEGEYV | Probe #265 | (FAM)-GHEGEYVGK(CPQ2)-PEG2-kk-NH2 | 942 |
| 266 | WSPSFK | Probe #266 | (FAM)-GWSPSFKGK(CPQ2)-NH2 | 943 |
| 267 | HDEHWT | Probe #267 | (FAM)-GHDEHWTGK(CPQ2)-PEG2-kk-NH2 | 944 |
| 268 | YVW(Nle)RD | Probe #268 | (FAM)-GYVW(Nle)RDGK(CPQ2)-NH2 | 945 |
| 269 | (Nle)DP(Nle) | Probe #269 | (FAM)-G(Nle)DP(Nle)KFGK(CPQ2)-NH2 | 946 |
| | KF | | | |
| 270 | (Nle)R(Nle) | Probe #270 | (FAM)-G(Nle)R(Nle)FWDGK(CPQ2)-NH2 | 947 |
| | FWD | | | |
| 271 | DIAIT(Nle) | Probe #271 | (FAM)-GDIAIT(Nle)GK(CPQ2)-PEG2-k-NH2 | 948 |
| 272 | PI(Nle)RFH | Probe #272 | (FAM)-GPI(Nle)RFHGK(CPQ2)-PEG2-k-NH2 | 949 |
| 273 | VWQGYI | Probe #273 | (FAM)-GVWQGYIGK(CPQ2)-PEG2-k-NH2 | 950 |
| 274 | KK(Nle)SNP | Probe #274 | (FAM)-GKK(Nle)SNPGK(CPQ2)-PEG2-k-NH2 | 951 |
| 275 | GHPLSP | Probe #275 | (FAM)-GGHPLSPGK(CPQ2)-PEG2-kk-NH2 | 952 |
| 276 | VRQHKP | Probe #276 | (FAM)-GVRQHKPGK(CPQ2)-NH2 | 953 |
| 277 | AQNFYR | Probe #277 | (FAM)-GAQNFYRGK(CPQ2)-NH2 | 954 |
| 278 | VAGKSI | Probe #278 | (FAM)-GVAGKSIGK(CPQ2)-NH2 | 955 |
| 279 | LVGQVN | Probe #279 | (FAM)-GLVGQVNGK(CPQ2)-PEG2-k-NH2 | 956 |
| 280 | QVKHFT | Probe #280 | (FAM)-GQVKHFTGK(CPQ2)-PEG2-k-NH2 | 957 |
| 281 | QKSVVS | Probe #281 | (FAM)-GQKSVVSGK(CPQ2)-NH2 | 958 |
| 282 | Y(Nle)QEWL | Probe #282 | (FAM)-GY(Nle)QEWLGK(CPQ2)-PEG2-k- | 959 |
| | | | NH2 | |
| 283 | G(Nle)YIDE | Probe #283 | (FAM)-GG(Nle)YIDEGK(CPQ2)-PEG2-k-NH2 | 960 |
| 284 | NAGSKF | Probe #284 | (FAM)-GNAGSKFGK(CPQ2)-NH2 | 961 |
| 285 | EFVHNP | Probe #285 | (FAM)-GEFVHNPGK(CPQ2)-PEG2-kk-NH2 | 962 |
| 286 | WE(Nle)VKI | Probe #286 | (FAM)-GWE(Nle)VKIGK(CPQ2)-NH2 | 963 |
| 287 | WVGASH | Probe #287 | (FAM)-GWVGASHGK(CPQ2)-PEG2-kk-NH2 | 964 |
| 288 | ITTLY(Nle) | Probe #288 | (FAM)-GITTLY(Nle)GK(CPQ2)-PEG2-k-NH2 | 965 |
| 289 | GHIDEY | Probe #289 | (FAM)-GGHIDEYGK(CPQ2)-PEG2-kk-NH2 | 966 |
| 290 | KV(Nle)DYG | Probe #290 | (FAM)-GKV(Nle)DYGGK(CPQ2)-NH2 | 967 |
| 291 | QEKQT(Nle) | Probe #291 | (FAM)-GQEKQT(Nle)GK(CPQ2)-NH2 | 968 |
| 292 | EVGHEA | Probe #292 | (FAM)-GEVGHEAGK(CPQ2)-PEG2-kk-NH2 | 969 |
| 293 | AWEGQY | Probe #293 | (FAM)-GAWEGQYGK(CPQ2)-PEG2-k-NH2 | 970 |
| 294 | FLVQWT | Probe #294 | (FAM)-GFLVQWTGK(CPQ2)-PEG2-k-NH2 | 971 |
| 295 | SKWGYW | Probe #295 | (FAM)-GSKWGYWGK(CPQ2)-NH2 | 972 |
| 296 | TWIS(Nle)Q | Probe #296 | (FAM)-GTWIS(Nle)QGK(CPQ2)-PEG2-k-NH2 | 973 |
| 297 | VIDKDF | Probe #297 | (FAM)-GVIDKDFGK(CPQ2)-NH2 | 974 |
| 298 | VKFAIY | Probe #298 | (FAM)-GVKFAIYGK(CPQ2)-NH2 | 975 |
| 299 | HNQ(Nle)KS | Probe #299 | (FAM)-GHNQ(Nle)KSGK(CPQ2)-PEG2-k- | 976 |
| | | | NH2 | |
| 300 | QYVFF(Nle) | Probe #300 | (FAM)-GQYVFF(Nle)GK(CPQ2)-PEG2-k-NH2 | 977 |
| 301 | YNPRE(Nle) | Probe #301 | (FAM)-GYNPRE(Nle)GK(CPQ2)-NH2 | 978 |
| 302 | KHG(Nle)PE | Probe #302 | (FAM)-GKHG(Nle)PEGK(CPQ2)-PEG2-kk- | 979 |
| | | | NH2 | |
| 303 | WSREYW | Probe #303 | (FAM)-GWSREYWGK(CPQ2)-NH2 | 980 |
| 304 | IDRVDK | Probe #304 | (FAM)-GIDRVDKGK(CPQ2)-PEG2-kk-NH2 | 981 |
| 305 | GDRENSPK | Probe #305 | (FAM)-kkGDRENSPK(CPQ2)L-OH | 982 |
| | (CPQ2)L-OH | | | |
| 306 | GDRENSPLK | Probe #306 | (FAM)-kkGDRENSPLK(CPQ2)-OH | 983 |
| | (CPQ2)-OH | | | |
| 307 | NAGSKFK | Probe #307 | (FAM)-GNAGSKFK(CPQ2)Q-OH | 984 |
| | (CPQ2)Q-OH | | | |
| 308 | NAGSKFQK | Probe #308 | (FAM)-GNAGSKFQK(CPQ2)-OH | 985 |
| | (CPQ2)-OH | | | |
| 309 | GHLLGFYK | Probe #309 | (FAM)-kkGHLLGFYK(CPQ2)V-OH | 986 |
| | (CPQ2)V-OH | | | |
| 310 | GHLLGFYVK | Probe #310 | (FAM)-kkGHLLGFYVK(CPQ2)-OH | 987 |
| | (CPQ2)-OH | | | |
| 311 | GQEKQT(Nle) | Probe #311 | (FAM)-kkGQEKQT(Nle)K(CPQ2)(Nle)-OH | 988 |
| | K(CPQ2)(Nle)- | | | |
| | OH | | | |
| 312 | GQEKQT(Nle) | Probe #312 | (FAM)-kkGQEKQT(Nle)(Nle)K(CPQ2)-OH | 989 |
| | (Nle)K(CPQ2)- | | | |
| | OH | | | |
| 313 | kGDPFVVSK | Probe #313 | (FAM)-kGDPFVVSK(CPQ2)W-OH | 990 |
| | (CPQ2)W-OH | | | |
| 314 | kGDPFVVSW | Probe #314 | (FAM)-kGDPFVVSWK(CPQ2)-OH | 991 |
| | K(CPQ2)-OH | | | |
| 315 | NAYNEIK | Probe #315 | (FAM)-GNAYNEIK(CPQ2)R-OH | 992 |
| | (CPQ2)R-OH | | | |
| 316 | NAYNEIRK | Probe #316 | (FAM)-GNAYNEIRK(CPQ2)-OH | 993 |
| | (CPQ2)-OH | | | |
| 317 | V(Nle)RQSEK | Probe #317 | (FAM)-GV(Nle)RQSEK(CPQ2)N-OH | 994 |
| | (CPQ2)N-OH | | | |
| 318 | V(Nle)RQSEN | Probe #318 | (FAM)-GV(Nle)RQSENK(CPQ2) | 995 |
| | K(CPQ2)-OH | | | |
| 319 | YNPRE(Nle)K | Probe #319 | (FAM)-GYNPRE(Nle)K(CPQ2)I-OH | 996 |
| | (CPQ2)I-OH | | | |
| 320 | YNPRE(Nle)IK | Probe #320 | (FAM)-GYNPRE(Nle)IK(CPQ2)-OH | 997 |
| | (CPQ2)-OH | | | |
| 321 | EFVHNPK | Probe #321 | (FAM)-kGEFVHNPK(CPQ2)K-OH | 998 |
| | (CPQ2)K-OH | | | |
| 322 | EFVHNPKK | Probe #322 | (FAM)-kGEFVHNPKK(CPQ2)-OH | 999 |
| | (CPQ2)-OH | | | |
| 323 | KRVQFLK | Probe #323 | (FAM)-GKRVQFLK(CPQ2)H-OH | 1000 |
| | (CPQ2)H-OH | | | |
| 324 | KRVQFLHK | Probe #324 | (FAM)-GKRVQFLHK(CPQ2)-OH | 1001 |
| | (CPQ2)-OH | | | |
| 325 | LI(Nle)HKNK | Probe #325 | (FAM)-kGLI(Nle)HKNK(CPQ2)G-OH | 1002 |
| | (CPQ2)G-OH | | | |
| 326 | LI(Nle)HKNG | Probe #326 | (FAM)-kGLI(Nle)HKNGK(CPQ2)-OH | 1003 |
| | K(CPQ2)-OH | | | |
| 327 | WA(Nle)LYHK | Probe #327 | (FAM)-kkGWA(Nle)LYHK(CPQ2)S-OH | 1004 |
| | (CPQ2)S-OH | | | |
| 328 | WA(Nle)LYHS | Probe #328 | (FAM)-kkGWA(Nle)LYHSK(CPQ2)-OH | 1005 |
| | K(CPQ2)-OH | | | |
| 329 | AHDIVNK | Probe #329 | (FAM)-kkGAHDIVNK(CPQ2)Y-OH | 1006 |
| | (CPQ2)Y-OH | | | |
| 330 | AHDIVNYK | Probe #330 | (FAM)-kkGAHDIVNYK(CPQ2)-OH | 1007 |
| | (CPQ2)-OH | | | |
| 331 | SVFVIEK | Probe #331 | (FAM)-kGSVFVIEK(CPQ2)P-OH | 1008 |
| | (CPQ2)P-OH | | | |
| 332 | SVFVIEPK | Probe #332 | (FAM)-kGSVFVIEPK(CPQ2)-OH | 1009 |
| | (CPQ2)-OH | | | |
| 333 | PPSGLSK | Probe #333 | (FAM)-kGPPSGLSK(CPQ2)E-OH | 1010 |
| | (CPQ2)E-OH | | | |
| 334 | PPSGLSEK | Probe #334 | (FAM)-kGPPSGLSEK(CPQ2)-OH | 1011 |
| | (CPQ2)-OH | | | |
| 335 | RWYGGIK | Probe #335 | (FAM)-kkGRWYGGIK(CPQ2)F-OH | 1012 |
| | (CPQ2)F-OH | | | |
| 336 | RWYGGIFK | Probe #336 | (FAM)-kkGRWYGGIFK(CPQ2)-OH | 1013 |
| | P(CQ2)-OH | | | |
| 337 | QYVFF(Nle)K | Probe #337 | (FAM)-kGQYVFF(Nle)K(CPQ2)D-OH | 1014 |
| | (CPQ2)D-OH | | | |
| 338 | QYVFF(Nle)D | Probe #338 | (FAM)-kGQYVFF(Nle)DK(CPQ2)-OH | 1015 |
| | K(CPQ2)-OH | | | |
| 339 | FAKYYKK | Probe #339 | (FAM)-kGFAKYYKK(CPQ2)T-OH | 1016 |
| | (CPQ2)T-OH | | | |
| 340 | FAKYYKTK | Probe #340 | (FAM)-KGFAKYYKTK(CPQ2)-OH | 1017 |
| | (CPQ2)-OH | | | |
| 341 | QVKHFTK | Probe #341 | (FAM)-kGQVKHFTK(CPQ2)A-OH | 1018 |
| | (CPQ2)A-OH | | | |
| 342 | QVKHFTAK | Probe #342 | (FAM)-kGQVKHFTAK(CPQ2)-OH | 1019 |
| | (CPQ2)-OH | | | |
| 343 | APK(CPQ2)- | Probe #343 | FAM-APK(CPQ2)-OH | 1020 |
| | OH | | | |
| 344 | NH2- | Probe #344 | NH2-HK(FAM)DRENSPGK(CPQ2)-NH2 | 1021 |
| | HK(FAM)DRE | | | |
| | NSP | | | |
| 345 | NH2- | Probe #345 | NH2-K(FAM)HDRENSPGK(CPQ2)-NH2 | 1022 |
| | K(FAM)HDRE | | | |
| | NSP | | | |
| 346 | NH2- | Probe #346 | NH2-WK(FAM)NAGSKFGKK(CPQ2)-NH2 | 1023 |
| | WK(FAM)NA | | | |
| | GSKF | | | |
| 347 | NH2- | Probe #347 | NH2-K(FAM)WNAGSKFGKK(CPQ2)-NH2 | 1024 |
| | K(FAM)WNA | | | |
| | GSKF | | | |
| 348 | NH2- | Probe #348 | NH2-SK(FAM)HLLGFYGKK(CPQ2)-NH2 | 1025 |
| | SK(FAM)HLL | | | |
| | GFY | | | |
| 349 | NH2- | Probe #349 | NH2-K(FAM)SHLLGFYGKK(CPQ2)-NH2 | 1026 |
| | K(FAM)SHLL | | | |
| | GFY | | | |
| 350 | NH2- | Probe #350 | NH2-KK(FAM)QEKQT(Nle)GK(CPQ2)-NH2 | 1027 |
| | KK(FAM)QEK | | | |
| | QT(Nle) | | | |
| 351 | NH2- | Probe #351 | NH2-K(FAM)KQEKQT(Nle)GK(CPQ2)-NH2 | 1028 |
| | K(FAM)KQEK | | | |
| | QT(Nle) | | | |
| 352 | NH2- | Probe #352 | NH2-GK(FAM)DPFVVSGK(CPQ2)-NH2 | 1029 |
| | GK(FAM)DPF | | | |
| | VVS | | | |
| 353 | NH2- | Probe #353 | NH2-K(FAM)GDPFVVSGK(CPQ2)-NH2 | 1030 |
| | K(FAM)GDPF | | | |
| | VVS | | | |
| 354 | NH2- | Probe #354 | NH2-PK(FAM)NAYNEIGK(CPQ2)-NH2 | 1031 |
| | PK(FAM)NAY | | | |
| | NEI | | | |
| 355 | NH2- | Probe #355 | NH2-K(FAM)PNAYNEIGK(CPQ2)-NH2 | 1032 |
| | K(FAM)PNAY | | | |
| | NEI | | | |
| 356 | NH2- | Probe #356 | NH2-DK(FAM)V(Nle)RQSEGKK(CPQ2)-NH2 | 1033 |
| | DK(FAM)V | | | |
| | (Nle)RQSE | | | |
| 357 | NH2- | Probe #357 | NH2-K(FAM)DV(Nle)RQSEGKK(CPQ2)-NH2 | 1034 |
| | K(FAM)DV | | | |
| | (Nle)RQSE | | | |
| 358 | NH2- | Probe #358 | NH2-EK(FAM)YNPRE(Nle)GkK(CPQ2)-NH2 | 1035 |
| | EK(FAM)YNP | | | |
| | RE(Nle) | | | |
| 359 | NH2- | Probe #359 | NH2-K(FAM)EYNPRE(Nle)GkK(CPQ2)-NH2 | 1036 |
| | K(FAM)EYNP | | | |
| | RE(Nle) | | | |
| 360 | NH2- | Probe #360 | NH2-TK(FAM)EFVHNPGkK(CPQ2)-NH2 | 1037 |
| | TK(FAM)EFV | | | |
| | HNP | | | |
| 361 | NH2- | Probe #361 | NH2-K(FAM)TEFVHNPGkK(CPQ2)-NH2 | 1038 |
| | K(FAM)TEFV | | | |
| | HNP | | | |
| 362 | NH2- | Probe #362 | NH2-QK(FAM)KRVQFLGK(CPQ2)-NH2 | 1039 |
| | QK(FAM)KRV | | | |
| | QFL | | | |
| 363 | NH2- | Probe #363 | NH2-K(FAM)QKRVQFLGK(CPQ2)-NH2 | 1040 |
| | K(FAM)QKRV | | | |
| | QFL | | | |
| 364 | NH2- | Probe #364 | NH2-YK(FAM)LI(Nle)HKNGK(CPQ2)-NH2 | 1041 |
| | YK(FAM)LI | | | |
| | (Nle)HKN | | | |
| 365 | NH2- | Probe #365 | NH2-K(FAM)YLI(Nle)HKNGK(CPQ2)-NH2 | 1042 |
| | K(FAM)YLI | | | |
| | (Nle)HKN | | | |
| 366 | NH2- | Probe #366 | NH2-FK(FAM)WA(Nle)LYHGkK(CPQ2)-NH2 | 1043 |
| | FK(FAM)WA | | | |
| | (Nle)LYH | | | |
| 367 | NH2- | Probe #367 | NH2-K(FAM)FWA(Nle)LYHGkK(CPQ2)-NH2 | 1044 |
| | K(FAM)FWA | | | |
| | (Nle)LYH | | | |
| 368 | NH2- | Probe #368 | NH2-IK(FAM)AHDIVNGkK(CPQ2)-NH2 | 1045 |
| | IK(FAM)AHDI | | | |
| | VN | | | |
| 369 | NH2- | Probe #369 | NH2-K(FAM)IAHDIVNGKK(CPQ2)-NH2 | 1046 |
| | K(FAM)IAHDI | | | |
| | VN | | | |
| 370 | NH2- | Probe #370 | NH2-VK(FAM)SVFVIEGK(CPQ2)-NH2 | 1047 |
| | VK(FAM)SVF | | | |
| | VIE | | | |
| 371 | NH2- | Probe #371 | NH2-K(FAM)VSVFVIEGK(CPQ2)-NH2 | 1048 |
| | K(FAM)VSVF | | | |
| | VIE | | | |
| 372 | NH2- | Probe #372 | NH2-(Nle)K(FAM)PPSGLSGK(CPQ2)-NH2 | 1049 |
| | (Nle)K(FAM)P | | | |
| | PSGLS | | | |
| 373 | NH2- | Probe #373 | NH2-K(FAM)(Nle)PPSGLSGK(CPQ2)-NH2 | 1050 |
| | K(FAM)(Nle)P | | | |
| | PSGLS | | | |
| 374 | NH2- | Probe #374 | NH2-LK(FAM)RWYGGIGKK(CPQ2)-NH2 | 1051 |
| | LK(FAM)RWY | | | |
| | GGI | | | |
| 375 | NH2- | Probe #375 | NH2-K(FAM)LRWYGGIGKK(CPQ2)-NH2 | 1052 |
| | K(FAM)LRWY | | | |
| | GGI | | | |
| 376 | NH2- | Probe #376 | NH2-NK(FAM)QYVFF(Nle)GK(CPQ2)-NH2 | 1053 |
| | NK(FAM)QYV | | | |
| | FF(Nle) | | | |
| 377 | NH2- | Probe #377 | NH2-K(FAM)NQYVFF(Nle)GK(CPQ2)-NH2 | 1054 |
| | K(FAM)NQYV | | | |
| | FF(Nle) | | | |
| 378 | NH2- | Probe #378 | NH2-AK(FAM)FAKYYKGK(CPQ2)-NH2 | 1055 |
| | AK(FAM)FAK | | | |
| | YYK | | | |
| 379 | NH2- | Probe #379 | NH2-K(FAM)AFAKYYKGK(CPQ2)-NH2 | 1056 |
| | K(FAM)AFAK | | | |
| | YYK | | | |
| 380 | NH2- | Probe #380 | NH2-RK(FAM)QVKHFTGK(CPQ2)-NH2 | 1057 |
| | RK(FAM)QVK | | | |
| | HFT | | | |
| 381 | NH2- | Probe #381 | NH2-K(FAM)RQVKHFTGK(CPQ2)-NH2 | 1058 |
| | K(FAM)RQVK | | | |
| | HFT | | | |
| 382 | NH2- | Probe #382 | NH2-K(FAM)PPK(CPQ2)-NH2 | 1059 |
| | K(FAM)PP | | | |
| 383 | kpilffrlk | Probe #383 | 5FAM-GkpilffrlkGK(CPQ2)-PEG2-kk-NH2 | 1060 |
| 384 | LRR | Probe #384 | Boc-Leu-Arg-Arg-AMC | 1061 |
| 385 | R | Probe #385 | Arg-AMC | 1062 |
| 386 | VR | Probe #386 | Boc-Val-Arg-AMC | 1063 |
| 387 | RR | Probe #387 | Z-Arg-Arg-AMC | 1064 |
| 388 | GR | Probe #388 | Gly-Arg-AMC | 1065 |
| 389 | FR | Probe #389 | Z-Phe-Arg-AMC | 1066 |
| 390 | RGK | Probe #390 | Ac-Arg-Gly-Lys-AMC | 1067 |
| 391 | GGR | Probe #391 | Z-Gly-Gly-Arg-AMC | 1068 |
| 392 | F | Probe #392 | Glutaryl-Phe-AMC | 1069 |
| 393 | D | Probe #393 | H-Asp-AMC | 1070 |
| 394 | RR | Probe #394 | H-Arg-Arg-AMC | 1071 |
| 395 | R | Probe #395 | Z-Arg-AMC | 1072 |
| 396 | Bz-R | Probe #396 | Bz-Arg-AMC | 1073 |
| 397 | Bz-R | Probe #397 | Bz-Arg-AMC | 1073 |
| 398 | PR | Probe #398 | Z-Pro-Arg-AMC | 1074 |
| 399 | GPR | Probe #399 | Z-Gly-Pro-Arg-AMC | 1075 |
| 400 | LR | Probe #400 | Z-Leu-Arg-AMC | 1076 |
| 401 | PFR | Probe #401 | H-Pro-Phe-Arg-AMC | 1077 |
| 402 | LLR | Probe #402 | Z-Leu-Leu-Arg-AMC | 1078 |
| 403 | QRR | Probe #403 | Boc-Gln-Arg-Arg-AMC | 1079 |
| 404 | GR | Probe #404 | Glutaryl-Gly-Arg-AMC | 1080 |
| 405 | GRR | Probe #405 | Boc-Gly-Arg-Arg-AMC | 1081 |
| 406 | LRGG | Probe #406 | Z-Leu-Arg-Gly-Gly-AMC | 1082 |
| 407 | RLRGG | Probe #407 | 5-FAM-GRLRGGGK(CPQ2)-PEG2-kk-GC | 1083 |
| 408 | RELNGGAPI | Probe #408 | 5-FAM-GRELNGGAPIGK(CPQ2)-PEG2-kk- | 1084 |
| | | | GC | |
| 409 | TSAVLQSGFR | Probe #409 | 5-FAM-GTSAVLQSGFRKGK(CPQ2)-PEG2- | 1085 |
| | K | | kk-GC | |
| 410 | SGVTFQGKF | Probe #410 | 5-FAM-GSGVTFQGKFKKGK(CPQ2)-PEG2- | 1086 |
| | KK | | kk-GC | |
| 411 | AAFA | Probe #411 | 5-FAM-GAAFAGK(CPQ2)-PEG2-kk-GC | 1087 |
| 412 | HGDQMAQKS | Probe #412 | 5FAM-GHGDQMAQKS-K(CPQ2)-PEG2- | 1088 |
| | | | DLys-DLys-GC-NH2 | |
| 413 | GPLGMR | Probe #413 | 5FAM-GGPLGMRG-K(CPQ2)-PEG2-DLys- | 1089 |
| | | | DLys-GC-NH2 | |
| 414 | FFLAQA- | Probe #414 | 5FAM-GFFLAQA-HomoPhe-RSK-K(CPQ2)- | 1090 |
| | HomoPhe-RSK | | PEG2-DLys-DLys-GC-NH2 | |
| 415 | AHAVSRIRIY | Probe #415 | 5FAM-GAHAVSRIRIYLLPAK-K(CPQ2)- | 1091 |
| | LLPAK | | PEG2-DLys-DLys-GC-NH2 | |
| 416 | PLALWAR | Probe #416 | 5FAM-GPLALWAR-K(CPQ2)-PEG2-DLys- | 1092 |
| | | | DLys-GC-NH2 | |
| 417 | PLA- | Probe #417 | 5FAM-GPLA-C(OMeBzl)-WAR-K(CPQ2)- | 1093 |
| | C(OMeBzl)- | | PEG2-DLys-DLys-GC-NH2 | |
| | WAR | | | |
| 418 | APRWIQD | Probe #418 | 5FAM-GAPRWIQD-K(CPQ2)-PEG2-DLys- | 1094 |
| | | | DLys-GC-NH2 | |
| 419 | LREQQRLKS | Probe #419 | 5FAM-GLREQQRLKS-K(CPQ2)-PEG2-DLys- | 1095 |
| | | | DLys-GC-NH2 | |
| 420 | EFPIYVFLPA | Probe #420 | 5FAM-GEFPIYVFLPAKK-K(CPQ2)-PEG2- | 1096 |
| | KK | | DLys-DLys-GC-NH2 | |
| 421 | GAANLVRGG | Probe #421 | 5FAM-GGAANLVRGG-K(CPQ2)-PEG2- | 1097 |
| | | | DLys-DLys-GC-NH2 | |
| 422 | GYAELRMG | Probe #422 | 5FAM-GGYAELRMGG-K(CPQ2)-PEG2- | 1098 |
| | | | DLys-DLys-GC-NH2 | |
| 423 | AAGAMFLEA | Probe #423 | 5FAM-GAAGAMFLEA-K(CPQ2)-PEG2- | 1099 |
| | | | DLys-DLys-GC-NH2 | |
| 424 | LGGSGQRGR | Probe #424 | (FAM)-GLGGSGQRGRKALEG-K(CPQ2)- | 1100 |
| | KALE | | (PEG2)-DLys-DLys-GC | |
| 425 | LGGSGHYGR | Probe #425 | (FAM)-GLGGSGHYGRSGLEG-K(CPQ2)- | 1101 |
| | SGLE | | (PEG2)-DLys-DLys-GC | |
| 426 | YGRS | Probe #426 | (FAM)-GYGRSG-K(CPQ2)-(PEG2)-DLys- | 1102 |
| | | | DLys-GC | |
| 427 | FRGRK | Probe #427 | (FAM)-GFRGRKG-K(CPQ2)-(PEG2)-DLys- | 1103 |
| | | | DLys-GC | |
| 428 | DRRKKLTQ | Probe #428 | (FAM)-GDRRKKLTQG-K(CPQ2)-(PEG2)- | 1104 |
| | | | DLys-DLys-GC | |
| 429 | HPGGPQ | Probe #429 | (FAM)-GHPGGPQG-K(CPQ2)-(PEG2)-DLys- | 1105 |
| | | | DLys-GC | |
| 430 | KLRFSKQ | Probe #430 | (FAM)-GKLRFSKQG-K(CPQ2)-(PEG2)-DLys- | 1106 |
| | | | DLys-GC | |
| 431 | AIKFFSAQ | Probe #431 | (FAM)-GAIKFFSAQG-K(CPQ2)-(PEG2)- | 1107 |
| | | | DLys-DLys-GC | |
| 432 | AIKFFVRQ | Probe #432 | (FAM)-GAIKFFVRQG-K(CPQ2)-(PEG2)- | 1108 |
| | | | DLys-DLys-GC | |
| 433 | RPPGFSAFK | Probe #433 | (FAM)-GRPPGFSAFKG-K(CPQ2)-(PEG2)- | 1109 |
| | | | DLys-DLys-GC | |
| 434 | FAP-QLS | Probe #434 | (FAM)-GFAP-QLSG-K(CPQ2)-(PEG2)-DLys- | 1110 |
| | | | DLys-GC | |
| 435 | FAA-QMA | Probe #435 | (FAM)-GFAA-QMAG-K(CPQ2)-(PEG2)-DLys- | 1111 |
| | | | DLys-GC | |
| 436 | GMP-ANQ | Probe #436 | (FAM)-GGMP-ANQG-K(CPQ2)-(PEG2)-DLys- | 1112 |
| | | | DLys-GC | |
| 437 | LSGRSDNH | Probe #437 | (FAM)-GLSGRSDNHG-K(CPQ2)-(PEG2)- | 1113 |
| | | | DLys-DLys-GC | |
| 438 | MAALITRPDF | Probe #438 | (FAM)-GMAALITRPDFG-K(CPQ2)-(PEG2)- | 1114 |
| | | | DLys-DLys-GC | |
| 439 | MAAAITRPRF | Probe #439 | (FAM)-GMAAAITRPRFG-K(CPQ2)-(PEG2)- | 1115 |
| | | | DLys-DLys-GC | |
| 440 | MAALIVRPDL | Probe #440 | (FAM)-GMAALIVRPDLG-K(CPQ2)-(PEG2)- | 1116 |
| | | | DLys-DLys-GC | |
| 441 | TSGPNQEQE | Probe #441 | (FAM)-GTSGPNQEQEG-K(CPQ2)-(PEG2)- | 1117 |
| | | | DLys-DLys-GC | |
| 442 | TAGPNQEQE | Probe #442 | (FAM)-GTAGPNQEQEG-K(CPQ2)-(PEG2)- | 1118 |
| | | | DLys-DLys-GC | |
| 443 | GPGPNQA | Probe #443 | (FAM)-GGPGPNQAG-K(CPQ2)-(PEG2)- | 1119 |
| | | | DLys-DLys-GC | |
| 444 | ASGPAGPA | Probe #444 | (FAM)-GASGPAGPAG-K(CPQ2)-(PEG2)- | 1120 |
| | | | DLys-DLys-GC | |
| 445 | ERGETGPSG | Probe #445 | (FAM)-GERGETGPSGG-K(CPQ2)-(PEG2)- | 1121 |
| | | | DLys-DLys-GC | |
| 446 | VSQELGQR | Probe #446 | (FAM)-GVSQELGQRG-K(CPQ2)-(PEG2)- | 1122 |
| | | | DLys-DLys-GC | |
| 447 | TGPPGYPTG | Probe #447 | (FAM)-GTGPPGYPTGG-K(CPQ2)-(PEG2)- | 1123 |
| | | | DLys-DLys-GC | |
| 448 | TRLPVYQ | Probe #448 | (FAM)-GTRLPVYQG-K(CPQ2)-(PEG2)-DLys- | 1124 |
| | | | DLys-GC | |
| 449 | RQARVVGG | Probe #449 | (FAM)-GRQARVVGGG-K(CPQ2)-(PEG2)- | 1125 |
| | | | DLys-DLys-GC | |
| 450 | RQRRVVGG | Probe #450 | (FAM)-GRQRRVVGGG-K(CPQ2)-(PEG2)- | 1126 |
| | | | DLys-DLys-GC | |
| 451 | RQARAVGG | Probe #451 | (FAM)-GRQARAVGGG-K(CPQ2)-(PEG2)- | 1127 |
| | | | DLys-DLys-GC | |
| 452 | RKRRGSRG | Probe #452 | (FAM)-GRKRRGSRGG-K(CPQ2)-(PEG2)- | 1128 |
| | | | DLys-DLys-GC | |
| 453 | KQSRKFVP | Probe #453 | (FAM)-GKQSRKFVPG-K(CPQ2)-(PEG2)- | 1129 |
| | | | DLys-DLys-GC | |
| 454 | VTGRS | Probe #454 | (FAM)-GVTGRSG-K(CPQ2)-(PEG2)-DLys- | 1130 |
| | | | DLys-GC | |
| 455 | LKSRVK | Probe #455 | (FAM)-GLKSRVKG-K(CPQ2)-(PEG2)-DLys- | 1131 |
| | | | DLys-GC | |
| 456 | GIGAVLKVLT | Probe #456 | (FAM)-GGIGAVLKVLTG-K(CPQ2)-(PEG2)- | 1132 |
| | | | DLys-DLys-GC | |
| 457 | GLPALISWIK | Probe #457 | (FAM)-GGLPALISWIKG-K(CPQ2)-(PEG2)- | 1133 |
| | | | DLys-DLys-GC | |
| 458 | SEVNLDAEF | Probe #458 | (FAM)-GSEVNLDAEFG-K(CPQ2)-(PEG2)- | 1134 |
| | | | DLys-DLys-GC | |
| 459 | EEKPICFFRL | Probe #459 | (FAM)-GEEKPICFFRLGKEG-K(CPQ2)- | 1135 |
| | GKE | | (PEG2)-DLys-DLys-GC | |
| 460 | EEKPILFFRLG | Probe #460 | (FAM)-GEEKPILFFRLGKEG-K(CPQ2)- | 1136 |
| | KE | | (PEG2)-DLys-DLys-GC | |
| 461 | APSSVIAA | Probe #461 | (FAM)-GAPSSVIAAG-K(CPQ2)-(PEG2)- | 1137 |
| | | | DLys-DLys-GC | |
| 462 | KKAKRNAL | Probe #462 | (FAM)-GKKAKRNALG-K(CPQ2)-(PEG2)- | 1138 |
| | | | DLys-DLys-GC | |
| 463 | WTNTSANYN | Probe #463 | (FAM)-GWTNTSANYNLG-K(CPQ2)-(PEG2)- | 1139 |
| | L | | DLys-DLys-GC | |
| 464 | RVRR | Probe #464 | (FAM)-GRVRRG-K(CPQ2)-(PEG2)-DLys- | 1140 |
| | | | DLys-GC | |
| 465 | ERTKR | Probe #465 | (FAM)-GERTKRG-K(CPQ2)-(PEG2)-DLys- | 1141 |
| | | | DLys-GC | |
| 466 | RYQIKPLKST | Probe #466 | (FAM)-GRYQIKPLKSTDEG-K(CPQ2)- | 1142 |
| | DE | | (PEG2)-DLys-DLys-GC | |
| 467 | WELRHQA- | Probe #467 | (FAM)-GWELRHQA-(Hfe)-RSKG-K(CPQ2)- | 1143 |
| | (Hfe)-RSK | | (PEG2)-DLys-DLys-GC | |
| 468 | SGAFK- | Probe #468 | (FAM)-GSGAFK-C(Me)-LKDGAGG- | 1144 |
| | C(Me)- | | K(CPQ2)-(PEG2)-DLys-DLys-GC | |
| | LKDGAG | | | |
| 469 | YVADGW | Probe #469 | (FAM)-GYVADGWG-K(CPQ2)-(PEG2)-DLys- | 1145 |
| | | | DLys-GC | |
| 470 | WEHDGW | Probe #470 | (FAM)-GWEHDGWG-K(CPQ2)-(PEG2)-DLys- | 1146 |
| | | | DLys-GC | |
| 471 | YVADAPV | Probe #471 | (FAM)-GYVADAPVG-K(CPQ2)-(PEG2)- | 1147 |
| | | | DLys-DLys-GC | |
| 472 | RPPGFSA | Probe #472 | (FAM)-GRPPGFSAG-K(CPQ2)-(PEG2)-DLys- | 1148 |
| | | | DLys-GC | |
| 473 | GSPAFLA | Probe #473 | (FAM)-GGSPAFLAG-K(CPQ2)-(PEG2)-DLys- | 1149 |
| | | | DLys-GC | |
| 474 | AGFSLPA | Probe #474 | (FAM)-GAGFSLPAG-K(CPQ2)-(PEG2)-DLys- | 1150 |
| | | | DLys-GC | |
| 475 | RWHTVGLR | Probe #475 | (FAM)-GRWHTVGLRWEG-K(CPQ2)-(PEG2)- | 1151 |
| | WE | | DLys-DLys-GC | |
| 476 | LEQ | Probe #476 | (FAM)-GLEQG-K(CPQ2)-(PEG2)-DLys-DLys- | 1152 |
| | | | GC | |
| 477 | RWPPMGLPW | Probe #477 | (FAM)-GRWPPMGLPWEG-K(CPQ2)-(PEG2)- | 1153 |
| | E | | DLys-DLys-GC | |
| 478 | RPKPVE | Probe #478 | (FAM)-GRPKPVEG-K(CPQ2)-(PEG2)-DLys- | 1154 |
| | | | DLys-GC | |
| 479 | IETD | Probe #479 | (FAM)-GIETDG-K(CPQ2)-(PEG2)-DLys- | 1155 |
| | | | DLys-GC | |
| 480 | VGPDFGR | Probe #480 | (FAM)-GVGPDFGRG-K(CPQ2)-(PEG2)-DLys- | 1156 |
| | | | DLys-GC | |
| 481 | GIEFDSGGC | Probe #481 | (FAM)-GGIEFDSGGCG-K(CPQ2)-(PEG2)- | 1157 |
| | | | DLys-DLys-GC | |
| 482 | GDFLRRV | Probe #482 | (FAM)-GGDFLRRVG-K(CPQ2)-(PEG2)-DLys- | 1158 |
| | | | DLys-GC | |
| 483 | AAL | Probe #483 | (FAM)-GAALG-K(CPQ2)-(PEG2)-DLys-DLys- | 1159 |
| | | | GC | |
| 484 | YATWSMIAA | Probe #484 | (FAM)-GYATWSMIAAHG-K(CPQ2)-(PEG2)- | 1160 |
| | H | | DLys-DLys-GC | |
| 485 | VIMWRLTVG | Probe #485 | (FAM)-GVIMWRLTVGTG-K(CPQ2)-(PEG2)- | 1161 |
| | T | | DLys-DLys-GC | |
| 486 | RRVLALQQE | Probe #486 | (FAM)-GRRVLALQQELG-K(CPQ2)-(PEG2)- | 1162 |
| | L | | DLys-DLys-GC | |
| 487 | LATWPLSGL | Probe #487 | (FAM)-GLATWPLSGLWG-K(CPQ2)-(PEG2)- | 1163 |
| | W | | DLys-DLys-GC | |
| 488 | NTPNWLVNA | Probe #488 | (FAM)-GNTPNWLVNAVG-K(CPQ2)-(PEG2)- | 1164 |
| | V | | DLys-DLys-GC | |
| 489 | SPLAQAVRSS | Probe #489 | (FAM)-GSPLAQAVRSSSRKG-K(CPQ2)- | 1165 |
| | SRK | | (PEG2)-DLys-DLys-GC | |
| 490 | QMPGRLSMA | Probe #490 | (FAM)-GQMPGRLSMAFG-K(CPQ2)-(PEG2)- | 1166 |
| | F | | DLys-DLys-GC | |
| 491 | PLGLR | Probe #491 | (FAM)-GPLGLRG-K(CPQ2)-(PEG2)-DLys- | 1167 |
| | | | DLys-GC | |
| 492 | QRANSIRVT | Probe #492 | (FAM)-GQRANSIRVTWG-K(CPQ2)-(PEG2)- | 1168 |
| | W | | DLys-DLys-GC | |
| 493 | PLAVR | Probe #493 | (FAM)-GPLAVRG-K(CPQ2)-(PEG2)-DLys- | 1169 |
| | | | DLys-GC | |
| 494 | LLAVPAANT | Probe #494 | (FAM)-GLLAVPAANTV G-K(CPQ2)- | 1170 |
| | V | | (PEG2)-DLys-DLys-GC | |
| 495 | GPQGLRGQ | Probe #495 | (FAM)-GGPQGLRGQG-K(CPQ2)-(PEG2)- | 1171 |
| | | | DLys-DLys-GC | |
| 496 | RTGLYLYNST | Probe #496 | (FAM)-GRTGLYLYNSTG-K(CPQ2)-(PEG2)- | 1172 |
| | | | DLys-DLys-GC | |
| 497 | RKKLTQSKF | Probe #497 | (FAM)-GRKKLTQSKFVGGAEG-K(CPQ2)- | 1173 |
| | VGGAE | | (PEG2)-DLys-DLys-GC | |
| 498 | KHYR | Probe #498 | (FAM)-GKHYRG-K(CPQ2)-(PEG2)-DLys- | 1174 |
| | | | DLys-GC | |
| 499 | QAR | Probe #499 | (FAM)-GQARG-K(CPQ2)-(PEG2)-DLys-DLys- | 1175 |
| | | | GC | |
| 500 | PRPFNYL | Probe #500 | (FAM)-GPRPFNYLG-K(CPQ2)-(PEG2)-DLys- | 1176 |
| | | | GC | |
| 501 | APFEMSA | Probe #501 | (FAM)-GAPFEMSAG-K(CPQ2)-(PEG2)-DLys- | 1177 |
| | | | DLys-GC | |
| 502 | APFEFSA | Probe #502 | (FAM)-GAPFEFSAG-K(CPQ2)-(PEG2)-DLys- | 1178 |
| | | | DLys-GC | |
| 503 | PLGFRV | Probe #503 | (FAM)-GPLGFRVG-K(CPQ2)-(PEG2)-DLys- | 1179 |
| | | | GC | |
| 504 | RPLALWRS | Probe #504 | (FAM)-GRPLALWRSG-K(CPQ2)-(PEG2)-GC | 1180 |
| 505 | RPLALEESQ | Probe #505 | (FAM)-GRPLALEESQG-K(CPQ2)-(PEG2)- | 1181 |
| | | | DLys-GC | |
| 506 | RPLALWRSQ | Probe #506 | (FAM)-GRPLALWRSQG-K(CPQ2)-(PEG2)- | 1182 |
| | | | GC | |
| 507 | RNALAVERT | Probe #507 | (FAM)-GRNALAVERTASG-K(CPQ2)- | 1183 |
| | AS | | (PEG2)-GC | |
| 508 | RPKPQQFW | Probe #508 | (FAM)-GRPKPQQFWG-K(CPQ2)-(PEG2)- | 1184 |
| | | | DLys-GC | |
| 509 | SGSNPYKYT | Probe #509 | (FAM)-SGSNPYKYTA-K(CPQ2)-(PEG2)- | 1185 |
| | A | | DLys-DLys-GC | |
| 510 | SGSNPYGYT | Probe #510 | (FAM)-SGSNPYGYTA-K(CPQ2)-(PEG2)- | 1186 |
| | A | | DLys-DLys-GC | |
| 511 | SGTLSELHTA | Probe #511 | (FAM)-SGTLSELHTA-K(CPQ2)-(PEG2)- | 1187 |
| | | | DLys-DLys-GC | |
| 512 | SGTISHLHTA | Probe #512 | (FAM)-SGTISHLHTA-K(CPQ2)-(PEG2)- | 1188 |
| | | | DLys-DLys-GC | |
| 513 | SG-(Orn)- | Probe #513 | (FAM)-SG-(Orn)-RSHP-(Hfe)-TLYTA- | 1189 |
| | RSHP-(Hfe)- | | K(CPQ2)-(PEG2)-DLys-GC | |
| | TLYTA | | | |
| 514 | SG-(Orn)- | Probe #514 | (FAM)-SG-(Orn)-RSHG-(Hfe)-FLYTA- | 1190 |
| | RSHG-(Hfe)- | | K(CPQ2)-(PEG2)-DLys-GC | |
| | FLYTA | | | |
| 515 | SGESLAYYTA | Probe #515 | (FAM)-SGESLAYYTA-K(CPQ2)-(PEG2)- | 1191 |
| | | | DLys-DLys-GC | |
| 516 | SGHMHAALT | Probe #516 | (FAM)-SGHMHAALTA-K(CPQ2)-(PEG2)- | 1192 |
| | A | | DLys-DLys-GC | |
| 517 | ILSR-(DIle)- | Probe #517 | (FAM)-GILSR-(DIle)-VGGG-K(CPQ2)- | 1193 |
| | VGG | | (PEG2)-DLys-GC | |
| 518 | ILS-(DArg)- | Probe #518 | (FAM)-GILS-(DArg)-(DIle)-(DVal)-GGG- | 1194 |
| | (DIle)- | | K(CPQ2)-(PEG2)-DLys-GC | |
| | (DVal)-GG | | | |
| 519 | RQRRALEK | Probe #519 | 5FAM-GRQRRALEKG-K(CPQ2)-PEG2-GC | 1195 |
| 520 | KPISLISS | Probe #520 | 5FAM-GKPISLISSG-K(CPQ2)-PEG2-GC | 1196 |
| 521 | QKGRYKQE | Probe #521 | 5FAM-GQKGRYKQEG-K(CPQ2)-PEG2-GC | 1197 |
| 522 | GPLGLRSW | Probe #522 | 5FAM-GGPLGLRSWK(CPQ2)-PEG2-C | 1198 |
| 523 | GPLGVRGK | Probe #523 | 5FAM-GGPLGVRGKK(CPQ2)-PEG2-C | 1199 |
| 524 | GfPRSGG | Probe #524 | 5FAM-GGfPRSGGGK(CPQ2)-PEG2-C | 1200 |
| 525 | Pyr | Probe #525 | Pyr-AMC | 1201 |
| 526 | SY | Probe #526 | H-Ser-Tyr-AMC | 1202 |
| 527 | GF | Probe #527 | H-Gly-Phe-AMC | 1203 |
| 528 | Y | Probe #528 | H-Tyr-AMC | 1204 |
| 529 | Cit | Probe #529 | H-Cit-AMC Hydrobromide salt | 1205 |
| 530 | GP | Probe #530 | Suc-Gly-Pro-AMC | 1206 |
| 531 | T | Probe #531 | H-Thr-AMC | 1207 |
| 532 | I | Probe #532 | H-Ile-AMC | 1208 |
| 533 | GA | Probe #533 | H-Gly-Ala-AMC hydrochloride salt | 1209 |
| 534 | Cys(Bzl) | Probe #534 | H-Cys(Bzl)-AMC | 1210 |
| 535 | A | Probe #535 | H-Ala-AMC | 1211 |
| 536 | K | Probe #536 | Ac-Lys-AMC acetate salt | 1212 |
| 537 | GLF | Probe #537 | MeOSuc-Gly-Leu-Phe-AMC | 1213 |
| 538 | L | Probe #538 | H-Leu-AMC | 1214 |
| 539 | VAN | Probe #539 | Z-Val-Ala-Asn-AMC | 1215 |
| 540 | AAA | Probe #540 | Suc-Ala-Ala-Ala-AMC | 1216 |
| 541 | K | Probe #541 | H-Lys-AMC acetate salt | 1217 |
| 542 | F | Probe #542 | H-Phe-AMC trifluoroacetate salt | 1218 |
| 543 | FSR | Probe #543 | Boc-Phe-Ser-Arg-AMC | 1219 |
| 544 | VVR | Probe #544 | Z-Val-Val-Arg-AMC hydrochloride salt | 1220 |
| 545 | KA | Probe #545 | H-Lys-Ala-AMC hydrochloride salt | 1221 |
| 546 | PR | Probe #546 | H-Pro-Arg-AMC hydrochloride salt | 1222 |
| 547 | MGP | Probe #547 | H-Met-Gly-Pro-AMC hydrochloride salt | 1223 |
| 548 | KP | Probe #548 | H-Lys-Pro-AMC hydrochloride salt | 1224 |
| 549 | QGR | Probe #549 | Boc-Gln-Gly-Arg-AMC hydrochloride salt | 1225 |
| 550 | Glu(OBzl)-AR | Probe #550 | Boc-Glu(OBzl)-Ala-Arg-AMC hydrochloride | 1226 |
| | | | salt | |
| 551 | WEHD | Probe #551 | Ac-Trp-Glu-His-Asp-AMC | 1227 |
| 552 | QAR | Probe #552 | Boc-Gln-Ala-Arg-AMC hydrochloride salt | 1228 |
| 553 | AAF | Probe #553 | H-Ala-Ala-Phe-AMC (free base) | 1229 |
| 554 | GPK | Probe #554 | Tos-Gly-Pro-Lys-AMC trifluoroacetate salt | 1230 |
| 555 | AAPM | Probe #555 | MeOSuc-Ala-Ala-Pro-Met-AMC | 1231 |
| 556 | AEPF | Probe #556 | Suc-Ala-Glu-Pro-Phe-AMC | 1232 |
| 557 | GG | Probe #557 | H-Gly-Gly-AMC hydrochloride salt | 1233 |
| 558 | VLK | Probe #558 | Boc-Val-Leu-Lys-AMC acetate salt | 1234 |
| 559 | EKK | Probe #559 | Boc-Glu-Lys-Lys-AMC acetate salt | 1235 |
| 560 | VPR | Probe #560 | Boc-Val-Pro-Arg-AMC hydrochloride salt | 1236 |
| 561 | GKR | Probe #561 | Boc-Gly-Lys-Arg-AMC hydrochloride salt | 1237 |
| 562 | Glu(OBzl)-GR | Probe #562 | Boc-Glu(OBzl)-Gly-Arg-AMC hydrochloride | 1238 |
| | | | salt | |
| 563 | LR | Probe #563 | Z-Leu-Arg-AMC hydrochloride salt | 1239 |
| 564 | AFK | Probe #564 | MeOSuc-Ala-Phe-Lys-AMC trifluoroacetate salt | 1240 |
| 565 | LGR | Probe #565 | Boc-Leu-Gly-Arg-AMC acetate salt | 1241 |
| 566 | PFR | Probe #566 | H-Pro-Phe-Arg-AMC acetate salt | 1242 |
| 567 | AAPV | Probe #567 | Suc-Ala-Ala-Pro-Val-AMC | 1243 |
| 568 | AFK | Probe #568 | H-Ala-Phe-Lys-AMC trifluoroacetate salt | 1244 |
| 569 | VKM | Probe #569 | Z-Val-Lys-Met-AMC acetate salt | 1245 |
| 570 | GPLGP | Probe #570 | Suc-Gly-Pro-Leu-Gly-Pro-AMC | 1246 |
| 571 | KQKER | Probe #571 | Ac-Lys-Gln-Lys-Leu-Arg-AMC trifluoroacetate | 1247 |
| | | | salt | |
| 572 | RVRR | Probe #572 | Boc-Arg-Val-Arg-Arg-AMC acetate salt | 1248 |
| 573 | IEGR | Probe #573 | Boc-Ile-Glu-Gly-Arg-AMC acetate salt | 1249 |
| 574 | GP | Probe #574 | H-Gly-Pro-AMC HBr | 1250 |
| 575 | AAPV | Probe #575 | MeOSuc-Ala-Ala-Pro-Val-AMC | 1251 |
| 576 | RPFHLLVY | Probe #576 | Suc-Arg-Pro-Phe-His-Leu-Leu-Val-Tyr-AMC | 1252 |
| | | | trifluoroacetate salt | |
| 577 | Anb-WS-Gnf- | Probe #577 | H-Anb-Trp-Ser-Gnf-Thr-Val-Phe-AMC | 1253 |
| | TVF | | | |
| 578 | HSSKLQ | Probe #578 | Mu-His-Ser-Ser-Lys-Leu-Gln-AMC | 1254 |
| 579 | RPY | Probe #579 | MeO-Succ-Arg-Pro-Tyr-AMC | 1255 |
| 580 | DRENSPK | Probe #580 | (ACC)-kkDRENSPK(Dnp)L | 1256 |
| | (Dnp)L-OH | | | |
| 581 | kkDRENSPLK | Probe #581 | (ACC)-kkDRENSPLK(Dnp) | 1257 |
| | (Dnp)-OH | | | |
| 582 | NAGSKFK | Probe #582 | (ACC)-NAGSKFK(Dnp)Q | 1258 |
| | (Dnp)Q-OH | | | |
| 583 | NAGSKFQK | Probe #583 | (ACC)-NAGSKFQK(Dnp) | 1259 |
| | (Dnp)-OH | | | |
| 584 | HLLGFYK | Probe #584 | (ACC)-kkHLLGFYK(Dnp)V | 1260 |
| | (Dnp)V-OH | | | |
| 585 | HLLGFYVK | Probe #585 | (ACC)-kkHLLGFYVK(Dnp) | 1261 |
| | (Dnp)-OH | | | |
| 586 | QEKQT(Nle)K | Probe #586 | (ACC)-kkQEKQT(Nle)K(Dnp)(Nle) | 1262 |
| | (Dnp)(Nle)-OH | | | |
| 587 | QEKQT(Nle) | Probe #587 | (ACC)-kkQEKQT(Nle)(Nle)K(Dnp) | 1263 |
| | (Nle)K(Dnp)- | | | |
| | OH | | | |
| 588 | DPFVVSK | Probe #588 | (ACC)-kDPFVVSK(Dnp)W | 1264 |
| | (Dnp)W-OH | | | |
| 589 | DPFVVSWK | Probe #589 | (ACC)-kDPFVVSWK(Dnp) | 1265 |
| | (Dnp)-OH | | | |
| 590 | NAYNEIK | Probe #590 | (ACC)-NAYNEIK(Dnp)R | 1266 |
| | (Dnp)R-OH | | | |
| 591 | NAYNEIRK | Probe #591 | (ACC)-NAYNEIRK(Dnp) | 1267 |
| | (Dnp)-OH | | | |
| 592 | V(Nle)RQSEK | Probe #592 | (ACC)-V(Nle)RQSEK(Dnp)N | 1268 |
| | (Dnp)N-OH | | | |
| 593 | V(Nle)RQSEN | Probe #593 | (ACC)-V(Nle)RQSENK(Dnp) | 1269 |
| | K(Dnp)-OH | | | |
| 594 | YNPRE(Nle)K | Probe #594 | (ACC)-YNPRE(Nle)K(Dnp)I | 1270 |
| | (Dnp)I-OH | | | |
| 595 | YNPRE(Nle)IK | Probe #595 | (ACC)-YNPRE(Nle)IK(Dnp) | 1271 |
| | (Dnp)-OH | | | |
| 596 | EFVHNPK | Probe #596 | (ACC)-kEFVHNPK(Dnp)K | 1272 |
| | (Dnp)K-OH | | | |
| 597 | EFVHNPKK | Probe #597 | (ACC)-KEFVHNPKK(Dnp) | 1273 |
| | (Dnp)-OH | | | |
| 598 | KRVQFLK | Probe #598 | (ACC)-KRVQFLK(Dnp)H | 1274 |
| | (Dnp)H-OH | | | |
| 599 | KRVQFLHK | Probe #599 | (ACC)-KRVQFLHK(Dnp) | 1275 |
| | (Dnp)-OH | | | |
| 600 | LI(Nle)HKNK | Probe #600 | (ACC)-KLI(Nle)HKNK(Dnp)G | 1276 |
| | (Dnp)G-OH | | | |
| 601 | LI(Nle)HKNG | Probe #601 | (ACC)-KLI(Nle)HKNGK(Dnp) | 1277 |
| | K(Dnp)-OH | | | |
| 602 | WA(Ne)LYHK | Probe #602 | (ACC)-kkWA(Nle)LYHK(Dnp)S | 1278 |
| | (Dnp)S-OH | | | |
| 603 | WA(Nle)LYHS | Probe #603 | (ACC)-kkWA(Nle)LYHSK(Dnp) | 1279 |
| | K(Dnp)-OH | | | |
| 604 | AHDIVNK | Probe #604 | (ACC)-kkAHDIVNK(Dnp)Y | 1280 |
| | (Dnp)Y-OH | | | |
| 605 | AHDIVNYK | Probe #605 | (ACC)-kkAHDIVNYK(Dnp) | 1281 |
| | (Dnp)-OH | | | |
| 606 | SVFVIEK | Probe #606 | (ACC)-kSVFVIEK(Dnp)P | 1282 |
| | (Dnp)P-OH | | | |
| 607 | SVFVIEPK | Probe #607 | (ACC)-kSVFVIEPK(Dnp) | 1283 |
| | (Dnp)-OH | | | |
| 608 | PPSGLSK | Probe #608 | (ACC)-kPPSGLSK(Dnp)E | 1284 |
| | (Dnp)E-OH | | | |
| 609 | PPSGLSEK | Probe #609 | (ACC)-kPPSGLSEK(Dnp) | 1285 |
| | (Dnp)-OH | | | |
| 610 | RWYGGIK | Probe #610 | (ACC)-kkRWYGGIK(Dnp)F | 1286 |
| | (Dnp)F-OH | | | |
| 611 | RWYGGIFK | Probe #611 | (ACC)-kkRWYGGIFK(Dnp) | 1287 |
| | (Dnp)-OH | | | |
| 612 | QYVFF(Nle)K | Probe #612 | (ACC)-kQYVFF(Nle)K(Dnp)D | 1288 |
| | (Dnp)D-OH | | | |
| 613 | QYVFF(Nle)D | Probe #613 | (ACC)-kQYVFF(Nle)DK(Dnp) | 1289 |
| | K(Dnp)-OH | | | |
| 614 | FAKYYKK | Probe #614 | (ACC)-kFAKYYKK(Dnp)T | 1290 |
| | (Dnp)T-OH | | | |
| 615 | FAKYYKTK | Probe #615 | (ACC)-kFAKYYKTK(Dnp) | 1291 |
| | (Dnp)-OH | | | |
| 616 | QVKHFTK | Probe #616 | (ACC)-kQVKHFTK(Dnp)A | 1292 |
| | (Dnp)A-OH | | | |
| 617 | QVKHFTAK | Probe #617 | (ACC)-kQVKHFTAK(Dnp) | 1293 |
| | (Dnp)-OH | | | |
| 618 | YVADAPK | Probe #618 | (ACC)-KYVADAPK(Dnp) | 1294 |
| | (Dnp)-OH | | | |
| 619 | KGISSQY | Probe #619 | ACC-GKGISSQYK(Dnp)-NH2 | 1295 |
| 620 | ALPALQN | Probe #620 | ACC-GALPALQNK(Dnp)-PEG2-Dlys-Dlys- | 1296 |
| | | | NH2 | |
| 621 | HRFRG | Probe #621 | ACC-GHRFRGK(Dnp)-NH2 | 1297 |
| 622 | APEEIMDQQ | Probe #622 | ACC-GAPEEIMDQQK(Dnp)-PEG2-Dlys-Dlys- | 1298 |
| | | | NH2 | |
| 623 | SRKSQQY | Probe #623 | ACC-GSRKSQQYK(Dnp)-NH2 | 1299 |
| 624 | SKGRSLI | Probe #624 | ACC-GSKGRSLIGK(Dnp)-NH2 | 1300 |
| 625 | FAQSIPK | Probe #625 | ACC-GFAQSIPKK(Dnp)-PEG2-Dlys-Dlys- | 1301 |
| | | | NH2 | |
| 626 | RQRRVVG | Probe #626 | ACC-GRQRRVVGGK(Dnp)-NH2 | 1302 |
| 627 | ERGETGPS | Probe #627 | ACC-GERGETGPSGK(Dnp)-NH2 | 1303 |
| 628 | ASGPSS | Probe #628 | ACC-GASGPSSGK(Dnp)-PEG2-Dlys-Dlys- | 1304 |
| | | | NH2 | |
| 629 | YRFR | Probe #629 | ACC-GYRFRGK(Dnp)-NH2 | 1305 |
| 630 | KLFSSKQ | Probe #630 | ACC-GKLFSSKQK(Dnp)-NH2 | 1306 |
| 631 | IVPRG | Probe #631 | ACC-GIVPRGK(Dnp)-NH2 | 1307 |
| 632 | IRRSSYFK | Probe #632 | ACC-GIRRSSYFKK(Dnp)-NH2 | 1308 |
| 633 | His(Bzl)-Tle- | Probe #633 | ACC-Gly-His(Bzl)-Tle-Pro-Ser-Asp-Met(O)- | 1309 |
| | PSD-Met(O) | | Gly-K(Dnp)-Gly-PEG2-Dlys-Dlys-NH2 | |
| 634 | Nva-IE-Oic- | Probe #634 | ACC-Nva-Ile-Glu-Oic-Asp-Phe-Gly-Arg- | 1310 |
| | DFGR | | Lys(Dnp)-NH2 | |
| 635 | H-DThr- | Probe #635 | Ac-His-DThr-Phe(F5)-Arg-ACC | 1311 |
| | Phe(F5)-R | | | |
| 636 | Dap-Orn- | Probe #636 | Ac-Dap-Orn-Phe(3Cl)-Cys(MeOBzl)-ACC | 1312 |
| | Phe(3Cl)- | | | |
| | Cys(MeOBzl) | | | |
| 637 | Cha-L- | Probe #637 | Ac-Cha-Leu-hSer(Bzl)-Arg-ACC | 1313 |
| | hSer(Bzl)-R | | | |
| 638 | His(Bzl)-Tle- | Probe #638 | ACC-Gly-His(Bzl)-Tle-Pro-Ser-Asp-Met(O)- | 1309 |
| | PSD-Met(O) | | Gly-K(Dnp)-Gly-PEG2-Dlys-Dlys-NH2 | |
| 639 | hCha- | Probe #639 | Ac-hCha-Phe(guan)-Oic-Arg-ACC | 1314 |
| | Phe(guan)- | | | |
| | Oic-R | | | |
| 640 | Abu-Nle | Probe #640 | NH2-Abu-Nle(O-Bzl)-ACC | 1315 |
| | (O-Bzl) | | | |
| 641 | Nle(O-Bzl)- | Probe #641 | Ac-Nle(O-Bzl)-Met(0)2-Oic-Abu-ACC | 1316 |
| | Met(O)2-Oic- | | | |
| | Abu | | | |
| 642 | Dap-Orn- | Probe #642 | ACC-G-Dap-Orn-Phe(3Cl)-Cys(MeOBz)-G- | 1317 |
| | Phe(3C1)- | | K(Dnp)-NH2 | |
| | Cys(MeOBz) | | | |
| 643 | Cha-L-hSer-R | Probe #643 | ACC-Gly-Cha-Leu-hSer-Arg-Gly-K(Dnp)-NH2 | 1318 |
| 644 | FVT-Gnf-SW | Probe #644 | ACC-Phe-Val-Thr-Gnf-Ser-Trp-K(Dnp)-NH2 | 1319 |
| 645 | hCha- | Probe #645 | ACC-Gly-hCha-Phe(guan)-Oic-Arg-Gly- | 1320 |
| | Phe(guan)- | | K(Dnp)-NH2 | |
| | Oic-R | | | |
| 646 | Nle(OBz)- | Probe #646 | ACC-Gly-Nle(OBz)-Met(02)-Oic-Abu-Gly- | 1321 |
| | Met(02)-Oic- | | K(Dnp)-NH2 | |
| | Abu | | | |
| 647 | AIEPDSG | Probe #647 | 5FAM-GAIEPDSGG-Lys(CPQ2)-PEG2-Dlys- | 1322 |
| | | | Dlys-GC-NH2 | |
| 648 | AIEFDSG | Probe #648 | 5FAM-GAIEFDSGG-Lys(CPQ2)-Dlys-Dlys- | 1323 |
| | | | GC-NH2 | |
| 649 | AAEAISD | Probe #649 | 5FAM-GGAAEAISDAK(CPQ2)-kk-PEG2-C | 1324 |
| 650 | AGGAQMGA | Probe #650 | 5FAM-GGAGGAQMGAK(CPQ2)-kk-PEG2-C | 1325 |
| 651 | AQPDALNV | Probe #651 | 5FAM-GGAQPDALNVK(CPQ2)-kk-PEG2-C | 1326 |
| 652 | ATDVTTTP | Probe #652 | 5FAM-GGATDVTTTPK(CPQ2)-kk-PEG2-C | 1327 |
| 653 | DIVTVANA | Probe #653 | 5FAM-GGDIVTVANAK(CPQ2)-kk-PEG2-C | 1328 |
| 654 | DLGLKSVP | Probe #654 | 5FAM-GGDLGLKSVPK(CPQ2)-kk-PEG2-C | 1329 |
| 655 | DVMASNKR | Probe #655 | 5FAM-GGDVMASNKRK(CPQ2)-kk-PEG2-C | 1330 |
| 656 | ESDELNTI | Probe #656 | 5FAM-GGESDELNTIK(CPQ2)-kk-PEG2-C | 1331 |
| 657 | FHPLHSKI | Probe #657 | 5FAM-GGFHPLHSKIK(CPQ2)-kk-PEG2-C | 1332 |
| 658 | HARLVHV | Probe #658 | 5FAM-GGGHARLVHVK(CPQ2)-kk-PEG2-C | 1333 |
| 659 | HIANVERV | Probe #659 | 5FAM-GGHIANVERVK(CPQ2)-kk-PEG2-C | 1334 |
| 660 | KAAATQKK | Probe #660 | 5FAM-GGKAAATQKKK(CPQ2)-kk-PEG2-C | 1335 |
| 661 | LATASTMD | Probe #661 | 5FAM-GGLATASTMDK(CPQ2)-kk-PEG2-C | 1336 |
| 662 | LGPKGQT | Probe #662 | 5FAM-GGLGPKGQTGK(CPQ2)-kk-PEG2-C | 1337 |
| 663 | LSLPETGE | Probe #663 | 5FAM-GGLSLPETGEK(CPQ2)-kk-PEG2-C | 1338 |
| 664 | NLAGILKE | Probe #664 | 5FAM-GGNLAGILKEK(CPQ2)-kk-PEG2-C | 1339 |
| 665 | NPGMSEPV | Probe #665 | 5FAM-GGNPGMSEPVK(CPQ2)-kk-PEG2-C | 1340 |
| 666 | PFGCHAK | Probe #666 | 5FAM-GGPFGCHAKK(CPQ2)-kk-PEG2-C | 1341 |
| 667 | PLGLRWW | Probe #667 | 5FAM-GGPLGLRWWK(CPQ2)-kk-PEG2-C | 1342 |
| 668 | QMGVMQGV | Probe #668 | 5FAM-GGQMGVMQGVK(CPQ2)-kk-PEG2-C | 1343 |
| 669 | QTCKCSCK | Probe #669 | 5FAM-GGQTCKCSCKK(CPQ2)-kk-PEG2-C | 1344 |
| 670 | QWAGLVEK | Probe #670 | 5FAM-GGQWAGLVEKK(CPQ2)-kk-PEG2-C | 1345 |
| 671 | RPAVMTSP | Probe #671 | 5FAM-GGRPAVMTSPK(CPQ2)-kk-PEG2-C | 1346 |
| 672 | TLRELHLD | Probe #672 | 5FAM-GGTLRELHLDK(CPQ2)-kk-PEG2-C | 1347 |
| 673 | TPPPSQGK | Probe #673 | 5FAM-GGTPPPSQGKK(CPQ2)-kk-PEG2-C | 1348 |
| 674 | TSEDLVVQ | Probe #674 | 5FAM-GGTSEDLVVQK(CPQ2)-kk-PEG2-C | 1349 |
| 675 | VWAAEAIS | Probe #675 | 5FAM-GGVWAAEAISK(CPQ2)-kk-PEG2-C | 1350 |
| 676 | R | Probe #676 | H-R-AMC | 1351 |
| 677 | GC | Probe #677 | FAM-GGC-PEG8 | 1352 |

Nle = norleucine
K(FAM) = carboxy-fluorescein-L-lysine
HomoPhe = Hfe = L-homophenylalanine
Cys(OMeBzl) = C(OMeBzl) = S-para-methoxybenzyl cysteine
DIle = d-isoleucine
DArg = D-arginine
DVal = D-valine
Pyr = pyroglutamic acid
Cit = citrulline
C(Bzl) = S-benzyl-L-cysteine
Glu(OBzl) = benzyl-L-glutamate
Anb = amino-n-butyric acid
Gnf = guamidine-L-phenylalanine
K(Dnp) = dinitrobenzylation of lysine
His(Bzl) = benzyl-L-histidine
Tle = L-tert-leucine
Met(O) = L-methionine-sulfoxide
Bz = Benzoyl
Oic = L-octahydroindole-2-carboxylic acid
Nva = norvaline (click to see farther down list)
DThr = d-threonine
Phe(F5) = 2,3,4,5,6-pentafluoro-L-penylalanine
Phe(3Cl) = 3-chloro-L-phenylalanine
hSer(Bzl) = benzyl homoserine
hCha = homocyclohexylalnine
Phe(guan) = phenylalanine derivative with a
guanidine group in the para position
Nle(O-Bzl) = Nle(OBz) = benzyloxy-L-norleucine
Met(O)2 = L-methionine sulfone
Dap = 2,3-diaminopropionic acid
hSer = homoserine
Met(02) = methylsulfonylbutanoic acid
Abu = L-alpha-aminobutyric acid
Cha = L-cyclohexylalanine
Cys(Me) = L- Methyl cysteine
Orn = L-Ornithine
hF = L-Homophenylalanine
GABA = gamma aminobutyric acid
Pip = piperidine carboxylic acid
lower case = D-amino acids

In some embodiments, the peptide linkers described herein for endoproteases comprise the following design: XₘAYₙ or AXₙB, wherein respectively, A is a single amino acid and A and B are amino acid pairs recognized by a particular endoprotease, X and Y are any amino acid labeled or not with a reporter, and m, n are zero or any integer. This design is for exemplification only and should not be construed as the only possible design for the peptide linker.

In some embodiments, the peptide linkers described herein for exoproteases comprise the following design: XₘAYₙ, wherein A is amino acid pairs recognized by a particular exoprotease, X and Y are any amino acid labeled or not with a reporter, and n is zero or any integer. This design is for exemplification only and should not be construed as the only possible design for the peptide linker.

TABLE 2
Exemplary peptide linker designs.

| | | | | | | | | Critical |
|---|---|---|---|---|---|---|---|---|
| | | | | | | | | amino |
| amino | amino | amino | amino | amino | Example | | | acid |
| acid | acid | acid | acid | acid | probe | | Protease | (single |
| in P1′ | in P1 | in P2 | in P3 | in P4 | name | Example probe design | family | or pair) |
| | R/K | | | | Probe | (FAM)-GWYKTQYGK(CPQ2)- | Endo | Single |
| | | | | | #161 | NH2 | | |
| | R/K | | | | Probe | (FAM)-GFARRWGGK(CPQ2)- | Endo | Single |
| | | | | | #109 | PEG2-k-NH2 | | |
| | F/Y/L/W | | | | Probe | (FAM)- | Endo | Single |
| | | | | | #165 | GSYWP(Nle)QGK(CPQ2)- | | |
| | | | | | | PEG2-k-NH2 | | |
| | F/Y | | | | Probe | (FAM)-GFIY(Nle)PTGK | Endo | Single |
| | | | | | #140 | (CPQ2)-PEG2-k-NH2 | | |
| | P | | | | Probe | (FAM)-GTGPKGNGK(CPQ2)- | Endo | Single |
| | | | | | #148 | NH2 | | |
| F | K | | | | Probe | (FAM)- | Endo | Pair |
| | | | | | #217 | GWSKFW(Nle)GK(CPQ2) | | (AB) |
| D | G | | | | Probe | (FAM)-GKTGDARGK(CPQ2)- | Endo | Pair |
| | | | | | #194 | PEG2-k-NH2 | | (AB) |
| L | P | | | | Probe | (FAM)-GGHPLSPGK(CPQ2)- | Endo | Pair |
| | | | | | #275 | PEG2-kk-NH2 | | (AB) |
| | D | T/I/V | | | Probe | (FAM)-GVIDKDFGK(CPQ2)- | Endo | Pair |
| | | | | | #297 | NH2 | | (AB) |
| | R | K/R | | | Probe | (FAM)-GFARRWGGK(CPQ2)- | Endo | Pair |
| | | | | | #109 | PEG2-k-NH2 | | (AB) |
| S | R | | | | Probe | (FAM)-GPVRSTNGK(CPQ2)- | Endo | Pair |
| | | | | | #204 | NH2 | | (AB) |
| | D | | E | | Probe | (FAM)-GENDRLPGK(CPQ2)- | Endo | Pair |
| | | | | | #199 | NH2 | | (near |
| | | | | | | | | neighbor |
| | | | | | | | | AXB) |
| | D | | V | | Probe | (FAM)-GQWVDEDGK(CPQ2)- | Endo | Pair |
| | | | | | #248 | PEG2-k-NH2 | | (near |
| | | | | | | | | neighbor |
| | | | | | | | | AXXB) |
| | K/R at | | | | Probe | (FAM)-kGEFVHNPK(CPQ2)K- | Exo | Single |
| | C- | | | | #321 | OH | | |
| | terminus | | | | | | | |
| | K/R/H at | | | | Probe | (FAM)-GNAYNEIK(CPQ2)R- | Exo | Single |
| | C- | | | | #315 | OH | | |
| | terminus | | | | | | | |
| | W/G/F | | | | Probe | NH2- | Exo | Single |
| | at N- | | | | #346 | WK(FAM)NAGSKFGkK(CPQ2)- | | |
| | terminus | | | | | NH2 | | |
| | Q/K at | | | | Probe | NH2- | Exo | Single |
| | N- | | | | #362 | QK(FAM)KRVQFLGK(CPQ2)- | | |
| | terminus | | | | | NH2 | | |

In some embodiments, the linker comprises a carbohydrate. Tung et al. reported a conjugate of β-galactoside and 7-hydroxy-9H-(1,3-dichloro-9,9-dimethylacridin-2-one), which has far-red fluorescence properties after a cleavage by β-galactosidase. Tung C H, Zeng Q, Shah K, Kim D E, Schellingerhout D, Weissleder R. In vivo imaging of beta-galactosidase activity using far red fluorescent switch. Cancer Res. 2004 Mar. 1; 64 (5): 1579-83. Ho et al. reported combining β-galactosidase substrate with p-benzyloxycarbonyl as a self-immolative linker. β-D-Galactopyranoside, the substrate of β-galactosidase, was conjugated to an optical probe through a para-substituted benzyloxycarbonyl group (serves as a first self-immolative linker) and a glycine residue (serves as a quencher and a second self-immolative linker). Enzymatic cleavage of the β-D-Galactopyranoside triggered a series of spontaneous reactions that resulted in a release of optically active probe. Ho, N.-H., Weissleder, R. and Tung, C.-H. (2007), A Self-Immolative Reporter For β-Galactosidase Sensing. ChemBioChem, 8:560-566. Some carbohydrate linkers are commercially available.

In some embodiments, the linker comprises a nucleic acid. The effect of a DNA linker on the behavior of its conjugate both reduces the toxicity of the free drug by reducing its cell penetration, which is positive in case of premature deconjugation in the bloodstream and increases the off-target toxicity on low antigen-expressing cells, presumably due to nonspecific interaction of the nucleic acid-based linker with the cell surface. For example, in an antibody-drug conjugates, the antibody and drug can be non-covalently connected using complementary DNA linkers. Dovgan, I., Ehkirch, A., Lehot, V. et al. On the use of DNA as a linker in antibody-drug conjugates: synthesis, stability and in vitro potency. Sci Rep 10, 7691 (2020). Dovgan et al. disclosed a trastuzumab to be connected to monomethyl auristatin E (MMAE) through a 37-mer oligonucleotide.

In some embodiments, the linker comprises a lipid. In some embodiments, the linker comprises a phospholipid. The insertion of phospholipid groups between two fluorescent dyes or a dye/quencher pair allows the detection of phospholipase cleavage activity. In some embodiments, the linker comprises a phosphodiester. The insertion of phosphodiester groups between two fluorescent dyes or a dye/quencher pair allows the detection of phosphodiesterase cleavage activity. In some embodiments, the lipid is directly attached to the fluorophore: once the covalent bond between the lipid and fluorophore is cleaved, the increase of fluorescent activity allows for the detection of the enzyme presence.

In some embodiments, the linker comprises an ester. Ester groups are often cleaved by saponification. The reactivity of the ester to cleavage can be enhanced by the use of electron-withdrawing groups or stabilized by the use of auto-immolative spacers to precluded spontaneous hydrolysis. In chemical biology, ester-based cleavable compounds were initially used for protein purification and in structural biology. FRET-based probes were designed to image esterase activities.

In some embodiments, the linker comprises a glycoside. For example, cellulase enzymes deconstruct cellulose to glucose, and are often comprised of glycosylated linkers connecting glycoside hydrolases (GHs) to carbohydrate-binding modules (CBMs).

In some embodiments, the linker comprises a nucleophile/base sensitive linker. These can include, but are not limited to, halogen nucleophiles, oxygen nucleophiles, safety-catch linkers, thiol nucleophiles, nitrogen nucleophiles, and phenacyl ester derivatives.

In some embodiments, the linker is sensitive to activity from all enzyme families, including, but not limited to, oxidoreductases, transferases, hydrolases, lyases, isomerases, and ligases.

Fluoridolyzable linkers are widely used in organic chemistry as silicon-based protecting groups for alcohols. The high thermodynamic affinity of fluorine for silicon allows their removal in orthogonal and mild conditions using a fluorine source. In that reaction a fluoride ion reacts with silicon as nucleophilic species and the cleavage conditions depend on the steric hindrance of the silicon's alkyl group. Fluoride ions can also trigger bond cleavage due to their basic properties.

Oxygen nucleophiles include sulfone and ester linkers while safety-catch linkers allow greater control over the timing of the bond breakage, because the linker will remain stable until it is activated for cleavage by a chemical modification.

In secondary amine synthesis or solid phase synthesis, nitrobenzenesulfonamides are known to be cleaved with a thiol nucleophile, like b-mercaptoethanol. Cysteines can be modified by electron-deficient alkynes to form a vinyl sulfide linkage.

Displacement reactions involving a specific nitrogen species as a nucleophile can occur in mild cleavable conditions. These reactions can be classified into two groups; cleavage by aminolysis or exchange reaction. For aminolysis cleavage, examples include the cleavage of a malondialdehyde (MDA) indole derivative by either pyrrolidine or hydrazine, and the cleavage of an ester linker by hydroxylamine or hydrazine. Acylhydrazones44 and hydrazones45,156 can be used as cleavable linkers through transimination in a mildly acidic medium. An amine catalyst (e.g., aniline, p-anisidine or hydroxylamine) accelerates hydrolysis and enables the effective transition between stable and dynamic states, which is required for cleavage and exchange.

In some embodiments, the linker comprises a reduction sensitive linker. Reduction sensitive linkages have been used in chemical biology for a long time and it is a commonly used class of cleavable linker. Examples of cleavable linkers sensitive to reductive conditions include: nitroreductases, disulfide bridges and azo compounds. Karan et al. reported a fluorescent probe to detect nitroreductase. Sanu Karan, Mi Young Cho, Hyunseung Lee, Hwunjae Lee, Hye Sun Park, Mahesh Sundararajan, Jonathan L. Sessler, and Kwan Soo Hong. Near-Infrared Fluorescent Probe Activated by Nitroreductase for In Vitro and In Vivo Hypoxic Tumor Detection. Journal of Medicinal Chemistry 2021 64 (6), 2971-2981. In naturally occurring proteins, disulfide bridges generally play a role in maintaining the protein structure. They are known to be efficiently and rapidly cleaved by mild reducing agents like dithiothreitol (DTT), b-mercaptoethanol or tris(2-carboxyethyl) phosphine (TCEP). In chemical biology, disulfide bridges have been used in a wide range of applications including functional and structural proteomics, drug delivery, tumor imaging, DNA and protein-DNA complex purifications. The disulfide-based linker is commonly used due to its straightforward synthesis and rapid cleavage. Azo linkers are very appealing to chemical biologists since they are able to undergo cleavage following treatment with sodium dithionite, a mild and potentially bio-orthogonal reducing agent. The azo compound is reduced into two aniline moieties via an electrochemical reduction mechanism and this allows the use of reducing agents that are commonly used in many biological protocols, such as TCEP, DTT. In chemical biology, azo compounds have been used to cross-link proteins for over a decade and more recently for protein affinity purification.

In some embodiments, the linker comprises an electrophile/acid sensitive linker. Acid sensitive linkers can be combined with other type of linkers. For example, a first β-galactosidase cleavage of the β-D-Galactopyranoside triggers the self-immolation of a benzyloxycarbonyl group, resulting in a release of optically active probe. Ho, N.-H., Weissleder, R. and Tung, C.-H. (2007), A Self-Immolative Reporter For β-Galactosidase Sensing. ChemBioChem, 8:560-566. Two different modes of electrophilic cleavage are used in chemical biology: acidic sensitive linkers that are sensitive to proton sources, and alkyl 2-(diphenylphosphino)benzoate derivatives sensitive to azide compounds. Proton sensitive bonds are among the most frequently used cleavable functions in organic chemistry; illustrated by the development of the BOC group which protects amines, or the Merrifield resin used in solid phase synthesis. In organic chemistry, the cleavage conditions that can be tolerated are very flexible regarding the acids” reagents, solvents, temperatures and pH. In contrast, biocompatible acid linkers must be responsive to minor changes in pH. Strong acidic conditions can lead to the denaturation of proteins and DNA. Biocompatible acid cleavable linkers are chosen for their instability near physiological pH and are often different from the classical protecting groups, which are cleaved with strong acids. Chemical reactions that can break or form bonds in water can be used as the basis of a linker, for example the Staudinger ligation. This reaction is proceeded by the nucleophilic attack of an alkyl 2-(diphenylphosphino)benzoate derivative on an azide, to form an aza-ylide intermediate. Then the ester traps the aza-ylide, which leads to the formation of an amide. In this process, the ester acts as a cleavable linker, and the azide as a bioorthogonal chemical agent, which guarantees a chemoselective and bioorthogonal cleavage.

In some embodiments, the linker comprises a metal linker. In some embodiments, the linker may be a metal cleavable linker. Organometallic compounds are used to catalyze the modification of proteins containing non-natural amino acids, but their use as cleavage reagent in chemical biology has only been reported a few times. The allyl function is a commonly used protecting group for alcohols in organic synthesis and it is also used as a linker in DNA sequencing by synthesis Metal cleavable linkers were also used in the design of peptide nucleic acids (PNAs), which were developed for enzyme-independent DNA/RNA hybridization methods.

In some embodiments, the linker comprises an oxidation sensitive linker. Sodium periodate is undoubtedly the most frequently used biocompatible oxidizing agent due to its ability to cleave vicinal diols to form two aldehydes compounds. One example of this type of linker consists of a vicinal diol with a tartaric acid spacer and two functional groups at both ends. Selenium based linkers also contain cleavable bonds sensitive to oxidizing agents, such as sodium periodate or N-chlorobenzenesulfonamide immobilized on polystyrene beads (iodo-beads). The trigger agent oxidizes the labile bond to selenium oxide, which is then cleaved directly via intramolecular b-elimination or rearrangement.

### Reporter

In some aspects, the synthetic molecule described herein comprises a reporter. The reporter as described herein can be in any structure that may be capable of being detected by any method, including but not limited to fluorescent detection, spectroscopic detection, immunological detection or imaging detection. In some embodiments, the reporter may be a fluorescent label, a mass tag or a nucleic acid barcode.

In some embodiments, the reporter may be a fluorescent label. Labels, tags and probes containing small compounds such as florescence can be used to label proteins and nucleic acids. Bio-affinity towards other molecules (biotin, digoxygenin), enzymatic (AP, HRP) or chemiluminescent (esters or acridine) can be used as well. Genetically encoded markers like the fluorescent proteins of the GFP family have become a reporter of choice for gene expression studies and protein localization. In combination with subcellular tags, GFP can be used to label subcellular structures like synapses allowing novel approaches to study developmental processes like synapse formation. Other fluorescent labels include but are not limited to small organic dyes and lipophilic dyes. The fluorescence label may serve itself as the activity substrate without addition of linkers.

Some reporters are “internally quenched”, and thus do not require a quencher, wherein the cleavage of a bond linking the internally quenched fluorophore to the substrate linker directly yields a fluorescent molecule. Many described probes for proteases, esterases, peroxidases and others function this way.

In some embodiments, the reporter comprises a mass tag. Mass tag reagents are designed to enable identification and quantitation of proteins in different samples using mass spectrometry (MS). Mass tagging reagents within a set typically have the same nominal mass (i.e., are isobaric) and chemical structure composed of an amine-reactive NHS ester group, a spacer arm (mass normalizer), and a mass reporter.

In some embodiments, the reporter comprises a nucleic acid barcode. For example, DNA barcoding is a system for species identification focused on the use of a short, standardized genetic region acting as a “barcode” in a similar way that Universal Product Codes are used by supermarket scanners to distinguish commercial products.

In some embodiments, the reporter can be detected using a ligand binding assay. A ligand binding assay often involves a detection step, such as an ELISA, including fluorescent, colorimetric, bioluminescent and chemiluminescent ELISAs, a paper test strip or lateral flow assay, or a bead-based fluorescent assay. In some embodiments, a paper-based ELISA test can be used to detect the cleaved reporter in the fluid sample. The paper-based ELISA may be created inexpensively, such as by reflowing wax deposited from a commercial solid ink printer to create an array of test spots on a single piece of paper. When the solid ink is heated to a liquid or semi-liquid state, the printed wax permeates the paper, creating hydrophobic barriers. The space between the hydrophobic barriers may then be used as individual reaction wells. The ELISA assay may be performed by drying the detection antibody on the individual reaction wells, constituting test spots on the paper, followed by blocking and washing steps. Fluid from a sample taken from the subject may then be added to the test spots. Then, for example, a streptavidin alkaline phosphate (ALP) conjugate may be added to the test spots, as the detection antibody. Bound ALP may then be exposed to a color reacting agent, such as BCIP/NBT (5-bromo-4-chloro-3″-indolyphosphate p-toluidine salt/nitro-blue tetrazolium chloride), which causes a purple colored precipitate, indicating presence of the reporter.

In some embodiments, the reporter can be detected using volatile organic compounds. Volatile organic compounds can be detected by analysis platforms such as gas chromatography instrument, a breathalyzer, a mass spectrometer, or use of optical or acoustic sensors. Gas chromatography can be used to detect compounds that can be vaporized without decomposition (e.g., volatile organic compounds). A gas chromatography instrument includes a mobile phase (or moving phase) that is a carrier gas, for example, an inert gas such as helium or an unreactive gas such as nitrogen, and a stationary phase that is a microscopic layer of liquid or polymer on an inert solid support, inside a piece of glass or metal tubing called a column. The column is coated with the stationary phase and the gaseous compounds analyzed interact with the walls of the column, causing them to elute at different times (i.e., have varying retention times in the column). Compounds may be distinguished by their retention times.

Mass spectrometry and enrichment/chromatography methods can be used to separate and distinguish/detect cleaved from intact reporters used in the present invention based on differences in mass and or presence of a label. For example, enzymatic reactions can result in the fragmentation of a parent molecule resulting in a mass shift of the starting substrate, this can be exploited in different chromatography/enrichment methods such as size exclusion chromatography and affinity enrichments. In mass spectrometry, a sample is ionized, for example by bombarding it with electrons. The sample may be a solid, liquid, or gas. By ionizing the sample, some of the sample's molecules are broken into charged fragments. These ions may then be separated according to their mass-to-charge ratio. This is often performed by accelerating the ions and subjecting them to an electric or magnetic field, where ions having the same mass-to-charge ratio will undergo the same amount of deflection. When deflected, the ions may be detected by a mechanism capable of detecting charged particles, for example, an electron multiplier. The detected results may be displayed as a spectrum of the relative abundance of detected ions as a function of the mass-to-charge ratio. The molecules in the sample can then be identified by correlating known masses, such as the mass of an entire molecule to the identified masses or through a characteristic fragmentation pattern.

When the reporter includes a nucleic acid, the reporter may be detected by various sequencing methods known in the art, for example, traditional Sanger sequencing methods or by next-generation sequencing (NGS). NGS generally refers to non-Sanger-based high throughput nucleic acid sequencing technologies, in which many (i.e., thousands, millions, or billions) of nucleic acid strands can be sequenced in parallel. Examples of such NGS sequencing includes platforms produced by Illumina (e.g., HiSeq, MiSeq, NextSeq, MiniSeq, and iSeq 100), Pacific Biosciences (e.g., Sequel and RSII), and Ion Torrent by ThermoFisher (e.g., Ion S5, Ion Proton, Ion PGM, and Ion Chef systems). It is understood that any suitable NGS sequencing platform may be used for NGS to detect nucleic acid of the detectable analyte as described herein.

Analysis can be performed directly on the biological sample or the detectable cleaved reporters may be purified to some degree first. For example, a purification step may involve isolating the detectable analyte from other components in the biological sample. Purification may include methods such as affinity chromatography. The isolated or purified detectable analyte does not need to be 100% pure or even substantially pure prior to analysis. Detecting the cleaved reporters may provide a qualitative assessment (e.g., whether the detectable cleaved reporters, and thus the predetermined protease is present or absent) or a quantitative assessment (e.g., the amount of the detectable cleaved reporters present) to indicate a comparative activity level of the predetermined proteases in the fluid sample. The quantitative value may be calculated by any means, such as, by determining the percent relative amount of each fraction present in the sample. Methods for making these types of calculations are known in the art.

The cleaved reporters can be detected by any detection method suitable for the particular reporter. In some aspects, the detection method comprises fluorescent detection, spectroscopic detection, mass spectrometry, immunological detection or imaging detection. In some aspects, the detection method comprises fluorescence resonance energy transfer (FRET).

In some embodiments, the detection method comprises spectroscopic detection. Spectroscopic methods of detection are employed in ion chromatography (IC) and are second only to conductivity detection in their frequency of usage. These methods can be divided broadly into the categories of molecular spectroscopic techniques and atomic spectroscopic techniques. Molecular spectroscopy includes UV-visible spectrophotometry, refractive index measurements, and photoluminescence techniques (fluorescence and phosphorescence). Atomic spectroscopy includes atomic emission spectroscopy (using various excitation sources) and atomic absorption spectroscopy. Many of the spectroscopic detection methods can operate in a direct or indirect mode. The definitions of these terms are the same as those used to describe the electrochemical detection modes. That is, direct spectroscopic detection results when the solute ion has a greater value of the measured detection parameter than does the eluent ion. Indirect detection results when the reverse is true.

In some embodiments, the detection method comprises mass spectrometry. Mass spectrometry (MS) is an analytical technique that is used to measure the mass-to-charge ratio of ions. The results are typically presented as a mass spectrum, a plot of intensity as a function of the mass-to-charge ratio.

In some embodiments, the detection method comprises fluorescence resonance energy transfer (FRET). FRET (Fluorescence Resonance Energy Transfer) is a distance dependent dipole-dipole interaction without the emission of a photon, which results in the transfer of energy from an initially excited donor molecule to an acceptor molecule. It allows the detection of molecular interactions in the nanometer range. FRET peptides are labeled with a donor molecule and an acceptor (quencher) molecule. In most cases, the donor and acceptor pairs are two different dyes. The transferred energy from a fluorescent donor is converted into molecular vibrations if the acceptor is a non-fluorescent dye (quencher). When the FRET is terminated (by separating donor and acceptor), an increase of donor fluorescence can be detected. When both the donor and acceptor dyes are fluorescent, the transferred energy is emitted as light of longer wavelength so that the intensity ratio change of donor and acceptor fluorescence can be measured. In order for efficient FRET quenching to take place, the fluorophore and quencher molecules must be close to each other (approximately 10-100 Å) and the absorption spectrum of the quencher must overlap with the emission spectrum of the fluorophore.

### Spacer

In some aspects, the synthetic molecule described herein comprises a spacer. In some cases, the linker comprises a first spacer. In some cases, the peptide sequence comprises a second spacer. In some embodiments, the spacer is a self-immolative spacer. In some embodiments, the self-immolative spacer comprise a disulfide, a p-amino benzyl alcohol, an a-quinone methide spacer, a hetheroaminebifuncional disulfide, a thiol-based pirydazinediones, a p-aminebenzyloxycarbonyl, a dipeptide, a Gly-Pro, a L-Phe-Sar, a trans-cyclooctene tetrazine, a ortho Hydroxy-protected Aryl sulfate, a phosphoramidate-based spacer, a hydroxybenzyl, a trimethyl carbamate, a quinone methide-based spacer, a cyclizing spacer, a Trimethyl lock, a 2-amino methyl piperidine or an ethylene diamine derived cyclizing spacer. Gonzaga et al. Perspective about self-immolative drug delivery systems. Journal of Pharmaceutical Sciences 109 (2020) 3262-3281.

Cleavage of the linker by a predetermined protease or enzyme makes the self-immolative spacer dissociate from the precipitating fluorescent or non-fluorescent reporter, thereby resulting in a detectable signal. The cleavable linker of the plurality of probes/molecules may be cleavable by a predetermined endoprotease in the body fluid sample resulting in auto immolation and reporter release or results in a protease substrate that can be cleaved by a predetermined exopeptidase. In some embodiments, the predetermined exopeptidase is added to the body fluid sample. In some embodiments, the predetermined exopeptidase cleaves the protease substrate, thereby causing the self-immolative spacer to dissociate from the precipitating fluorescent reporter, thereby resulting in a detectable signal.

### Detection Methods and Reporter

In some aspects, the synthetic molecule described herein comprises a reporter. The reporter as described herein can be in any structure that is capable of being detected by any method, including, but not limited to fluorescent detection, spectroscopic detection, immunological detection or imaging detection. In some embodiments, the reporter can be a fluorescent label, a mass tag or a nucleic acid barcode.

In some embodiments, the reporter comprises a fluorescent label. Labels, tags and probes containing small compounds such as florescence can be used to label proteins and nucleic acids. Bio-affinity towards other molecules (biotin, digoxygenin), enzymatic (AP, HRP) or chemiluminescent (esters or acridine) can be used as well. Genetically encoded markers like the fluorescent proteins of the GFP family have become a reporter of choice for gene expression studies and protein localization. In combination with subcellular tags, GFP can be used to label subcellular structures like synapses allowing novel approaches to study developmental processes like synapse formation. Other fluorescent labels include, but are not limited to, small organic dyes and lipophilic dyes. The fluorescence label may serve itself as the activity substrate without addition of linkers.

Some reporters are “internally quenched”, and thus do not require a quencher, wherein the cleavage of a bond linking the internally quenched fluorophore to the substrate linker directly yields a fluorescent molecule. Many described probes for proteases, esterases, peroxidases and others function this way.

In some embodiments, the reporter comprises a mass tag. Mass tag reagents are designed to enable identification and quantitation of proteins in different samples using mass spectrometry (MS). Mass tagging reagents within a set typically have the same nominal mass (i.e., are isobaric) and chemical structure composed of an amine-reactive NHS ester group, a spacer arm (mass normalizer), and a mass reporter.

In some embodiments, the reporter comprises a nucleic acid barcode. For example, DNA barcoding is a system for species identification focused on the use of a short, standardized genetic region acting as a “barcode” in a similar way that Universal Product Codes are used by supermarket scanners to distinguish commercial products.

In some embodiments, the reporter can be detected using a ligand binding assay. A ligand binding assay often involves a detection step, such as an ELISA, including fluorescent, colorimetric, bioluminescent and chemiluminescent ELISAs, a paper test strip or lateral flow assay, or a bead-based fluorescent assay. In some embodiments, a paper-based ELISA test can be used to detect the cleaved reporter in the fluid sample. The paper-based ELISA can be created inexpensively, such as by reflowing wax deposited from a commercial solid ink printer to create an array of test spots on a single piece of paper. When the solid ink is heated to a liquid or semi-liquid state, the printed wax permeates the paper, creating hydrophobic barriers. The space between the hydrophobic barriers may then be used as individual reaction wells. The ELISA assay may be performed by drying the detection antibody on the individual reaction wells, constituting test spots on the paper, followed by blocking and washing steps. Fluid from a sample taken from the subject may then be added to the test spots. Then, for example, a streptavidin alkaline phosphate (ALP) conjugate may be added to the test spots, as the detection antibody. Bound ALP may then be exposed to a color reacting agent, such as BCIP/NBT (5-bromo-4-chloro-3″-indolyphosphate p-toluidine salt/nitro-blue tetrazolium chloride), which causes a purple-colored precipitate, indicating presence of the reporter.

In some embodiments, the reporter can be detected using volatile organic compounds. Volatile organic compounds may be detected by analysis platforms such as gas chromatography instrument, a breathalyzer, a mass spectrometer, or use of optical or acoustic sensors. Gas chromatography may be used to detect compounds that can be vaporized without decomposition (e.g., volatile organic compounds). A gas chromatography instrument includes a mobile phase (or moving phase) that is a carrier gas, for example, an inert gas such as helium or an unreactive gas such as nitrogen, and a stationary phase that is a microscopic layer of liquid or polymer on an inert solid support, inside a piece of glass or metal tubing called a column. The column is coated with the stationary phase and the gaseous compounds analyzed interact with the walls of the column, causing them to elute at different times (i.e., have varying retention times in the column). Compounds may be distinguished by their retention times.

Mass spectrometry and enrichment/chromatography methods can be used to separate and distinguish/detect cleaved from intact reporters used in the present invention based on differences in mass and or presence of a label. For example, enzymatic reactions can result in the fragmentation of a parent molecule resulting in a mass shift of the starting substrate, this can be exploited in different chromatography/enrichment methods such as size exclusion chromatography and affinity enrichments. In mass spectrometry, a sample is ionized, for example by bombarding it with electrons. The sample may be a solid, liquid, or gas. By ionizing the sample, some of the sample's molecules are broken into charged fragments. These ions may then be separated according to their mass-to-charge ratio. This is often performed by accelerating the ions and subjecting them to an electric or magnetic field, where ions having the same mass-to-charge ratio will undergo the same amount of deflection. When deflected, the ions can be detected by a mechanism capable of detecting charged particles, for example, an electron multiplier. The detected results may be displayed as a spectrum of the relative abundance of detected ions as a function of the mass-to-charge ratio. The molecules in the sample can then be identified by correlating known masses, such as the mass of an entire molecule to the identified masses or through a characteristic fragmentation pattern.

When the reporter includes a nucleic acid, the reporter can be detected by various sequencing methods known in the art, for example, traditional Sanger sequencing methods or by next-generation sequencing (NGS). NGS generally refers to non-Sanger-based high throughput nucleic acid sequencing technologies, in which many (i.e., thousands, millions, or billions) of nucleic acid strands can be sequenced in parallel. Examples of such NGS sequencing includes platforms produced by Illumina (e.g., HiSeq, MiSeq, NextSeq, MiniSeq, and iSeq 100), Pacific Biosciences (e.g., Sequel and RSII), and Ion Torrent by ThermoFisher (e.g., Ion S5, Ion Proton, Ion PGM, and Ion Chef systems). It is understood that any suitable NGS sequencing platform may be used for NGS to detect nucleic acid of the detectable analyte as described herein.

Analysis can be performed directly on the biological sample, or the detectable cleaved reporters can be purified to some degree first. For example, a purification step may involve isolating the detectable analyte from other components in the biological sample. Purification may include methods such as affinity chromatography. The isolated or purified detectable analyte does not need to be 100% pure or even substantially pure prior to analysis. Detecting the cleaved reporters may provide a qualitative assessment (e.g., whether the detectable cleaved reporters, and thus the predetermined protease is present or absent) or a quantitative assessment (e.g., the amount of the detectable cleaved reporters present) to indicate a comparative activity level of the predetermined proteases in the fluid sample. The quantitative value can be calculated by any means, such as, by determining the percent relative amount of each fraction present in the sample. Methods for making these types of calculations are known in the art.

The cleaved reporters can be detected by any detection method that may be suitable for the particular reporter. In some aspects, the detection method comprises fluorescent detection, spectroscopic detection, mass spectrometry, immunological detection or imaging detection. In some aspects, the detection method may be fluorescence resonance energy transfer (FRET).

In some embodiments, the detection method comprises spectroscopic detection. Spectroscopic methods of detection are very commonly employed in ion chromatography (IC) and are second only to conductivity detection in their frequency of usage. These methods can be divided broadly into the categories of molecular spectroscopic techniques and atomic spectroscopic techniques. Molecular spectroscopy includes UV-visible spectrophotometry, refractive index measurements, and photoluminescence techniques (fluorescence and phosphorescence). Atomic spectroscopy includes atomic emission spectroscopy (using various excitation sources) and atomic absorption spectroscopy. Many of the spectroscopic detection methods can operate in a direct or indirect mode. The definitions of these terms are the same as those used to describe the electrochemical detection modes. That is, direct spectroscopic detection results when the solute ion has a greater value of the measured detection parameter than does the eluent ion. Indirect detection results when the reverse is true.

In some embodiments, the detection method comprises mass spectrometry. Mass spectrometry (MS) is an analytical technique that is used to measure the mass-to-charge ratio of ions. The results are typically presented as a mass spectrum, a plot of intensity as a function of the mass-to-charge ratio.

In some embodiments, the detection method comprises fluorescence resonance energy transfer (FRET). FRET (Fluorescence Resonance Energy Transfer) is a distance dependent dipole-dipole interaction without the emission of a photon, which results in the transfer of energy from an initially excited donor molecule to an acceptor molecule. It allows the detection of molecular interactions in the nanometer range. FRET peptides are labeled with a donor molecule and an acceptor (quencher) molecule. In most cases, the donor and acceptor pairs are two different dyes. The transferred energy from a fluorescent donor is converted into molecular vibrations if the acceptor is a non-fluorescent dye (quencher). When the FRET is terminated (by separating donor and acceptor), an increase of donor fluorescence can be detected. When both the donor and acceptor dyes are fluorescent, the transferred energy is emitted as light of longer wavelength so that the intensity ratio change of donor and acceptor fluorescence can be measured. In order for efficient FRET quenching to take place, the fluorophore and quencher molecules must be close to each other (approximately 10-100 Å) and the absorption spectrum of the quencher must overlap with the emission spectrum of the fluorophore.

### Precipitating Fluorophore

In some aspects, the cleaved reporter comprises a precipitating fluorophore. In some embodiments, the precipitating fluorophore comprises HPQ, Cl-HPQ, HTPQ, HTPQA, HBPQ, or HQPQ.

In some embodiments, the precipitating fluorophore comprises HPQ, also known as 2-(2″-hydroxyphenyl)-4 (3H)-quinazolinone. HPQ is a small organic dye known for its classic luminescence mechanism through excited-state intramolecular proton transfer (ESIPT), shows strong light emission in the solid state, but no emission in solution. HPQ is found to be strictly insoluble in water and exhibits intense solid-state fluorescence similar to that of tetraphenyl ethylene. Moreover, its essential properties of insolubility and intense solid-state fluorescence can be countered and reversed, by prohibiting the establishment of an internal hydrogen bond between the imine nitrogen and phenolic hydroxyl group.

In some embodiments, the precipitating fluorophore comprises Cl-HPQ. Cl-HPQ is released when HPQF, a water soluble and non-fluorescent molecule, reacts with furin. Cl-HPQ starts to precipitate near the enzyme activity site, and the precipitates emit bright solid-state fluorescence with more than 60-fold fluorescence enhancement. Li et al. In Situ Imaging of Furin Activity with a Highly Stable Probe by Releasing of Precipitating Fluorochrome. Anal. Chem. 2018, 90, 19, 11680-11687.

In some embodiments, the precipitating fluorophore comprises HTPQ. HTPQ is found to be strictly insoluble in water and shows intense fluorescence in the solid state with maximum excitation and emission wavelengths at 410 nm and 550 nm respectively. This makes it far better suited to the use with a confocal microscope. The large Stokes shift of HTPQ contributes additional and highly desirable advantages: increased sensitivity, minimized background fluorescence and enhanced bioimaging contrast. Liu et al. In Situ Localization of Enzyme activity in Live Cells by a Molecular Probe Releasing a Precipitating Fluorochrome. Angew Chem Int Ed Engl. 2017 Sep. 18; 56 (39): 11788-11792.

In some embodiments, the precipitating fluorophore comprises HTPQA. HTPQA is another enzyme-responsive fluorogenic probe derived from HTPQ. When converted by ALP, the probe releases free HTPQ which starts to precipitate after a very short delay; the precipitate emits bright solid-state fluorescence with more than 100-fold fluorescence enhancement.

In some embodiments, the precipitating fluorophore comprises HBPQ. HBPQ is completely insoluble in water and shows strong yellow solid emission when excited with a 405 nm laser. Liu et al. Precipitated Fluorophore-Based Molecular Probe for In Situ Imaging of Aminopeptidase N in Living Cells and Tumors. Anal. Chem. 2021, 93, 16, 6463-6471, Publication Date: Apr. 14, 2021.

In some embodiments, the precipitating fluorophore comprises HQPQ. HQPQ is, a novel solid-state fluorophore that is insoluble in water. Li et al. Precipitated Fluorophore-Based Probe for Accurate Detection of Mitochondrial Analytes. Anal. Chem. 2021, 93, 4, 2235-2243. Publication Date: Jan. 5, 2021.

The precipitating and non-precipitating fluorophores can be separated from the enzyme substrate by a self-immolative substrate to stabilize the initial probe and ensure that the enzymatic cleavage is transduced via the immolative spacer into the formation of the precipitating fluorophore or the non-internally quenched soluble fluorophore.

### Fluorescent Quencher

In some aspects, the synthetic molecule described herein comprises a fluorescent quencher. The fluorescent quencher as described herein can be in any structure that is capable of decreasing the fluorescence intensity of a given substance. In some embodiments, the fluorescent quencher may be BHQ0, BHQ1, BHQ2, BHQ3, BBQ650, ATTO 540Q, ATTO 580Q, ATTO 612Q, CPQ2, QSY-21, QSY-35, QSY-7, QSY-9, DABCYL (4-([4′-dimethylamino)phenyl]azo)benzoyl), Dnp (2,4-dinitrophenyl) or Eclipse®.

In some embodiments, the fluorescent quencher comprises a BHQ quencher including, but not limited to, BHQ0, BHQ1, BHQ2, BHQ3, or BBQ650. BHQ, or black hole quencher, dyes work through a combination of FRET and static quenching to enable avoidance of the residual background signal common to fluorescing quenchers such as TAMRA, or low signal-to-noise ratio. The different types of BHQ dyes are used to quench different colored dyes with BHQ1 used to quench green and yellow dyes such as FAM, TET, or HEX and BHQ2 used for quenching orange and red dyes. BHQ dyes are true dark quenchers with no native emission due to their polyacromatic-azo backbone. Substituting electron-donating and withdrawing groups on the aromatic rings produces a complete series of quenchers with broad absorption curves that span the visible spectrum.

In some embodiments, the fluorescent quencher comprises an ATTO quencher including, but not limited to ATTO 540Q, ATTO 580Q, or ATTO 612Q. ATTO quenchers have characteristic properties of strong absorption (high extinction coefficient) and high photo-stability. ATTO quenchers are often utilized as fluorescent quenchers on amine-labeled nucleotides for FRET experiments.

In some embodiments, the fluorescent quencher comprises a CPQ2 quencher. The quencher CPQ2 is often used as a pair with the fluorescent donor 5-carboxylfluorescein.

In some embodiments, the fluorescent quencher comprises a QSY quencher including, but not limited to, QSY-21, QSY-35, QSY-7, or QSY-9. QSY probes are dark quenchers, substances that absorb excitation energy from a fluorophore and dissipate the energy as heat.

In some embodiments, the fluorescent quencher comprises DABCYL (4-([4′-dimethylamino)phenyl]azo)benzoyl). DABCYL is one of the most popular acceptors for developing FRET-based nucleic acid probes and protease substrates. DABCYL dyes are often paired with EDANS in FRET-based fluorescent probes. DABCYL has a broad and intense visible absorption but no fluorescence.

In some embodiments, the fluorescent quencher comprises Dnp (2,4-dinitrophenyl). Dnp is a stable quencher and its absorption spectrum does not change with pH, which makes this group a convenient marker for substrate quantitation in solutions.

In some embodiments, the fluorescent quencher comprises Eclipse®. Eclipse® is a non-fluorescent chromophore and a dark quencher often used in dual-labelled probes. As dark quenchers, Eclipse® absorbs energy without emitting fluorescence. Eclipse® has an absorption range from 390 nm to 625 nm and is capable of effective performance in a wide range of colored FRET probes.

### Body Fluid Sample

Determination of the disease or condition is based on the rate of formation or amount of the released reporter detected in a body fluid sample. In some embodiments, the body fluid sample comprises blood, serum, plasma, bone marrow fluid, lymphatic fluid, bile, amniotic fluid, mucosal fluid, saliva, urine, cerebrospinal fluid, synovial fluid, semen, ductal aspirate, feces, vaginal effluent, cyst fluid, tissue homogenate, tissue-derived fluid, lachrymal fluid and patient-derived cell line supernatant. In some embodiments, the body fluid sample comprises a rinse fluid. In some embodiments, the rinse fluid comprises a mouthwash rinse, a bronchioalveolar rinse, a lavage fluid, a hair wash rinse, a nasal spray effluent, a swab of any bodily surface, orifice or organ structure applied to saline or any media or any derivatives thereof.

In some embodiments, the body fluid sample comprises blood. Blood is a constantly circulating fluid providing the body with nutrition, oxygen, and waste removal. Blood is mostly liquid, with numerous cells and proteins suspended in it. Blood is made of several main factors including plasma, red blood cells, white blood cells, and platelets.

In some embodiments, the body fluid sample comprises a plasma. Plasma is the liquid that remains when clotting is prevented with the addition of an anticoagulant. Serum is the conventional term in the art for the fluid that remains when clotting factors are removed from plasma. In some embodiments, an anticoagulant is introduced to the body fluid sample. In some embodiments, the anticoagulant is introduced to the body fluid sample before the synthetic molecule. In some embodiments, the anticoagulant is introduced to the body fluid sample after the synthetic molecule. Anticoagulants are medicines that help prevent blood clots. Examples of anticoagulants include, but are not limited to, an ethylenediamine tetraacetic acid (EDTA), a citrate, a heparin, an oxalate, any salt, solvate, enantiomer, tautomer and geometric isomer thereof, or any mixtures thereof.

In some embodiments, the anticoagulant comprises EDTA. The main property of EDTA, a polyprotic acid containing four carboxylic acid groups and two amine groups with lone pair electrons, is the ability to chelate or complex metal ions in 1:1 metal-EDTA complexes. Owing to its strong complexation with metal ions that are cofactors for enzymes, EDTA is widely used as a sequestering agent to prevent some enzyme reactions from occurring. When blood is collected with no additives within an appropriate container (blood tube), it clots fairly quickly. As calcium ions are necessary for this process, the specific association between the carboxylic groups of EDTA and calcium is a reliable solution to prevent clotting, stabilizing whole blood in a fluid form, as required for some laboratory analyses. Moreover, EDTA showed optimal extended stabilization of blood cells and particles. Three EDTA formulations can be employed as anticoagulants: Na₂EDTA, K₂EDTA and K₃EDTA, choice of which mostly depends on the type of analyses to be performed.

In some embodiments, the anticoagulant comprises a citrate. Citrate (C6H7O7) is a small negatively charged molecule with a molecular weight of 191 Daltons. Citrate can be used as the anticoagulant of choice for stored blood products, typically as acid citrate dextrose (ACD), (3.22% citrate, 112.9 mmol/l citrate, 123.6 mmol/l glucose, 224.4 mmol/l sodium and 114.2 mmol/l hydrogen ions), or trisodium citrate (TCA) Na₃C₃H₅O(COO)₃, (4% TCA, 136 mmol/l citrate, 420 mmol/l sodium). Citrate chelates calcium, and at a concentration of 4-6 mmol/l with an ionized calcium of <0.2 mmol/l prevents activation of both coagulation cascades and platelets. As such, citrate has been the standard anticoagulant used by hematologists and blood transfusion services for stored blood products and also as an extracorporeal anticoagulant for centrifugal platelet and leucopheresis techniques and plasma exchange.

In some embodiments, the anticoagulant comprises a heparin. The molecular basis for the anticoagulant action of heparin lies in its ability to bind to and enhance the inhibitory activity of the plasma protein antithrombin against several serine proteases of the coagulation system, most importantly factors IIa (thrombin), Xa and IXa. Two major mechanisms underlie heparin's potentiation of antithrombin. The conformational changes induced by heparin binding cause both expulsion of the reactive loop and exposure of exosites of the surface of antithrombin, which bind directly to the enzyme target; and a template mechanism exists in which both inhibitor and enzyme bind to the same heparin molecule. The relative importance of these two modes of action varies between enzymes. In addition, heparin can act through other serine protease inhibitors such as heparin co-factor II, protein C inhibitor and tissue factor plasminogen inhibitor. The antithrombotic action of heparin in vivo, though dominated by anticoagulant mechanisms, is more complex, and interactions with other plasma proteins and cells play significant roles in the living vasculature.

In some embodiments, the anticoagulant comprises an oxalate. Sodium, potassium, ammonium, and lithium oxalates inhibit blood coagulation by forming insoluble complex with calcium. Potassium oxalate at concentration of 1-2 mg/ml of blood is widely used. Combined ammonium and/or potassium oxalate does not cause shrinkage of erythrocytes. It consists of three parts by weight of ammonium oxalate, which causes swelling of the erythrocytes, balanced by two parts of potassium oxalate which causes shrinkage. NH4+ & K+ oxalate mixture in the ratio of 3:2, and 2 mg/ml of blood is the required amount.

In some embodiments, the body fluid sample comprises bone marrow fluid. Bone marrow is found in the center of most bones and has many blood vessels. There are two types of bone marrow: red and yellow. Red marrow contains blood stem cells that can become red blood cells, white blood cells, or platelets. Yellow marrow is made mostly of fat.

In some embodiments, the body fluid sample comprises lymphatic fluid. Lymphatic fluid, also called lymph, is a collection of the extra fluid that drains from cells and tissues, that is not reabsorbed into the capillaries.

In some embodiments, the body fluid sample comprises bile. Bile is a digestive fluid produced by the liver and stored in the gallbladder. During bile reflux, digestive fluid backs up into the stomach and, in some cases, the esophagus.

In some embodiments, the body fluid sample comprises amniotic fluid. Amniotic fluid is a clear, slightly yellowish liquid that surrounds the unborn baby (fetus) during pregnancy. It is contained in the amniotic sac.

In some embodiments, the body fluid sample comprises mucosal fluid. Mucosal fluid, also called mucus, is a thick protective fluid that is secreted by mucous membranes and used to stop pathogens and dirt from entering the body. Mucus is also used to prevent bodily tissues from being dehydrated.

In some embodiments, the body fluid sample comprises saliva. Saliva is an extracellular fluid produced and secreted by salivary glands in the mouth.

In some embodiments, the body fluid sample comprises urine. Urine is a liquid by-product of metabolism in humans and in many other animals. Urine flows from the kidneys through the ureters to the urinary bladder.

In some embodiments, the body fluid sample comprises cerebrospinal fluid. Cerebrospinal fluid is a clear fluid that surrounds the brain and spinal cord. It cushions the brain and spinal cord from injury and also serves as a nutrient delivery and waste removal system for the brain.

In some embodiments, the body fluid sample comprises synovial fluid. Synovial fluid, also known as joint fluid, is a thick liquid located between your joints. The fluid cushions the ends of bones and reduces friction when joints are moved.

In some embodiments, the body fluid sample comprises semen. Semen is the male reproductive fluid which contains spermatozoa in suspension.

In some embodiments, the body fluid sample comprises ductal aspirate. Ductal aspirate, also known as ductal lavage, ductal fluid, or lavage fluid, is fluid collected from a duct, such as the milk duct of the breast.

In some embodiments, the body fluid sample comprises feces. Feces, also known as excrement or stool is waste matter discharged from the bowels after food has been digested.

In some embodiments, the body fluid sample comprises vaginal effluent. Vaginal effluent, also known as vaginal discharge, is a clear or whitish fluid that comes out of the vagina.

In some embodiments, the body fluid sample comprises lachrymal fluid. Lachrymal fluid, also known as lacrimal fluid, is secreted by the lacrimal glands to lubricate the eye and fight bacteria.

In some embodiments, the body fluid sample comprises tissue homogenate. A tissue homogenate is obtained through mechanical micro-disruption of fresh tissue and the cell membranes are mechanically permeabilized.

### Proteases and Other Agents

The synthetic molecule described herein comprises cleaved by a protease from the body fluid. In some embodiments, the protease comprises an endopeptidase or an exopeptidase.

In some embodiments, the protease comprises an endopeptidase. An endopeptidase is an enzyme which breaks peptide bonds other than terminal ones in a peptide chain.

In some embodiments, the protease comprises an exopeptidase. An exopeptidase is an enzyme that catalyzes the cleavage of the terminal or penultimate peptide bond; the process releases a single amino acid or dipeptide from the peptide chain.

In some embodiments, the protease comprises an A20 (TNFa-induced protein 3), an abhydrolase domain containing 4, an abhydrolase domain containing 12, an abhydrolase domain containing 12B, an abhydrolase domain containing 13, an acrosin, an acylaminoacyl-peptidase, a disintegrin and metalloproteinase (ADAM), an ADAM1a, an ADAM2 (Fertilin-b), an ADAM3B, an ADAM4, an ADAM4B, an ADAM5, an ADAM6, an ADAM7, an ADAM8, an ADAM9, an ADAM10, an ADAM11, an ADAM12 metalloprotease, an ADAM15, an ADAM17, an ADAM18, an ADAM19, an ADAM20, an ADAM21, an ADAM22, an ADAM23, an ADAM28, an ADAM29, an ADAM30, an ADAM32, an ADAM33, a disintegrin and metalloproteinase with thrombospondin motifs (ADAMTS), an ADAMTS1, an ADAMTS2, an ADAMTS3, an ADAMTS4, an ADAMTS5/11, an ADAMTS6, an ADAMTS7, an ADAMTS8, an ADAMTS9, an ADAMTS10, an ADAMTS12, an ADAMTS13, an ADAMTS14, an ADAMTS15, an ADAMTS16, an ADAMTS17, an ADAMTS18, an ADAMTS19, an ADAMTS20, an adipocyte-enh. binding protein 1, an Afg3-like protein 1, an Afg3-like protein 2, an airway-trypsin-like protease, an aminoacylase, an aminopeptidase A, an aminopeptidase B, an aminopeptidase B-like 1, an aminopeptidase MAMS/L-RAP, an aminopeptidase N, an aminopeptidase O, an aminopeptidase P homologue, an aminopeptidase P1, an aminopeptidase PILS, an aminopeptidase Q, an aminopeptidase-like 1, an AMSH/STAMBP, an AMSH-LP/STAMBPL1, an angiotensin-converting enzyme 1 (ACE1), an angiotensin-converting enzyme 2 (ACE2), an angiotensin-converting enzyme 3 (ACE3), an anionic trypsin (II), an apolipoprotein (a), an archaemetzincin-1, an archaemetzincin-2, an aspartoacylase, an aspartoacylase-3, an aspartyl aminopeptidase, an ataxin-3, an ataxin-3 like, an ATP/GTP binding protein 1, an ATP/GTP binding protein-like 2, an ATP/GTP binding protein-like 3, an ATP/GTP binding protein-like 4, an ATP/GTP binding protein-like 5, an ATP23 peptidase, an autophagin-1, an autophagin-2, an autophagin-3, an autophagin-4, an azurocidin, or a combination hereof.

In some embodiments, the protease comprises a beta lactamase, a beta-secretase 1, a beta-secretase 2, a bleomycin hydrolase, a brain serine proteinase 2, a BRCC36 (BRCA2-containing complex, sub 3), a calpain, a calpain 1, a calpain 2, a calpain 3, a calpain 4, a calpain 5, a calpain 6, a calpain 7, a calpain 7-like, a calpain 8, a calpain 9, a calpain 10, a calpain 11, a calpain 12, a calpain 13, a calpain 14, a calpain 15 (Solh protein), or a combination hereof.

In some embodiments, the protease comprises a cysteine protease, a carboxypeptidase A1, a carboxypeptidase A2, a carboxypeptidase A3, a carboxypeptidase A4, a carboxypeptidase A5, a carboxypeptidase A6, a carboxypeptidase B, a carboxypeptidase D, a carboxypeptidase E, a carboxypeptidase M, a carboxypeptidase N, a carboxypeptidase O, a carboxypeptidase U, a carboxypeptidase X1, a carboxypeptidase X2, a carboxypeptidase Z, a carnosine dipeptidase 1, a carnosine dipeptidase 2, a caspase recruitment domain family, member 8, a caspase, a caspase-1, a caspase-2, a caspase-3, a caspase-4/11, a caspase-5, a caspase-6, a caspase-7, a caspase-8, a caspase-9, a caspase-10, a caspase-12, a caspase-14, a caspase-14-like, a casper/FLIP, a cathepsin, a cathepsin A (CTSA), a cathepsin B (CTSB), a cathepsin C (CTSC), a cathepsin D (CTSD), a cathepsin E (CTSE), a cathepsin F, a cathepsin G, a cathepsin H (CTSH), a cathepsin K (CTSK), a cathepsin L (CTSL), a cathepsin L2, a cathepsin O, a cathepsin S (CTSS), a cathepsin V (CTSV), a cathepsin W, a cathepsin Z (CTSZ), a cationic trypsin, a cezanne/OTU domain containing 7B, a cezanne-2, a CGI-58, a chymase, a chymopasin, a chymosin, a chymotrypsin B, a chymotrypsin C, a coagulation factor IXa, a coagulation factor VIIa, a coagulation factor Xa, a coagulation factor XIa, a coagulation factor XIIa, a collagenase 1, a collagenase 2, a collagenase 3, a complement protease C1r serine protease, a complement protease C1s serine protease, a complement C1r-homolog, a complement component 2, a complement component C1ra, a complement component C1sa, a complement factor B, a complement factor D, a complement factor D-like, a complement factor I, a COPS6, a corin, a CSN5 (JAB1), a cylindromatosis protein, a cytosol alanyl aminopep.-like 1, a cytosol alanyl aminopeptidase, or a combination hereof.

In some embodiments, the protease comprises a DDI-related protease, a DECYSIN, a Der1-like domain family, member 1, a Der1-like domain family, member 2, a Der1-like domain family, member 3, a DESC1 protease, a desert hedgehog protein, a desumoylating isopeptidase 1, a desumoylating isopeptidase 2, a dihydroorotase, a dihydropyrimidinase, a dihydropyrimidinase-related protein 1, a dihydropyrimidinase-related protein 2, a dihydropyrimidinase-related protein 3, a dihydropyrimidinase-related protein 4, a dihydropyrimidinase-related protein 5, a DINE peptidase, a dipeptidyl peptidase (DPP), a dipeptidyl peptidase (DPP1), a dipeptidyl-peptidase 4 (DPP4), a dipeptidyl-peptidase 6 (DPP6), a dipeptidyl-peptidase 8 (DPP8), a dipeptidyl-peptidase 9 (DPP9), a dipeptidyl-peptidase II, a dipeptidyl-peptidase III, a dipeptidyl-peptidase 10 (DPP10), a DJ-1, a DNA-damage inducible protein, a DNA-damage inducible protein 2, a DUB-1, a DUB-2, a DUB2a, a DUB2a-like, a DUB2a-like2, a DUB6, or a combination hereof.

In some embodiments, the protease comprises an enamelysin, an endopeptidase Clp, an endoplasmic reticulum metallopeptidase 1, an endothelin-converting enzyme 1, an endothelin-converting enzyme 2, an enteropeptidase, an epidermis-specific SP-like, an epilysin, an epithelial cell transforming sequence 2 oncogene-like, an epitheliasin, an epoxide hydrolase, an epoxyde hydrolase related protein, an eukar. translation initiation F3SF, an eukar. translation initiation F3SH, or a combination hereof.

In some embodiments, the protease comprises a Factor VII activating protease, a FACE-1/ZMPSTE24, a FACE-2/RCE1, a family with sequence similarity, member A1, a family with sequence similarity, member B1, a family with sequence similarity, member C1, a family with sequence similarity, A, a family with sequence similarity, B, a furin, or a combination hereof.

In some embodiments, the protease comprises a gamma-glutamyl hydrolase, a gamma-glutamyltransferase 1, a gamma-glutamyltransferase 2, a gamma-glutamyltransferase 5, a gamma-glutamyltransferase 6, a gamma-glutamyltransferase m−3, a gamma-glutamyltransferase-like 3, a GCDFP15, a gelatinase A, a gelatinase B, a Gln-fructose-6-P transamidase 1, a Gln-fructose-6-P transamidase 2, a Gln-fructose-6-P transamidase 3, a Gln-PRPP amidotransferase, a glutamate carboxypeptidase II, a glutaminyl cyclase, a glutaminyl cyclase 2, a glycosylasparaginase, a glycosylasparaginase-2, a granzyme, a granzyme A, a granzyme B, a granzyme H, a granzyme K, a granzyme M, a haptoglobin-1, or a combination hereof.

In some embodiments, the protease comprises a histone deacetylase (HDAC), a haptoglobin-related protein, a HAT-like 2, a HAT-like 3, a HAT-like 4, a HAT-like 5, a HAT-related protease, HSP90AA1? (a heat shock 90 kDa protein 1, alpha), HSP90AB1? (a heat shock 90 kDa protein 1, beta), a heat shock protein 75, a heat shock protein 90 kDa beta (Grp94), member 1/tumor rejection antigen (gp96), a hepatocyte growth factor, a hepsin, a HetF-like, a HGF activator, a hGPI8, a Hin-1/OTU domain containing 4, a homologue ICEY, a HP43.8KD, a HTRA1 serine protease, a HTRA2, a HTRA3, a HTRA4, a hyaluronan-binding ser-protease, an implantation serine protease 2, an indian hedgehog protein, an insulysin, an intestinal serine protease 1, an josephin-1, an josephin-2, or a combination hereof.

In some embodiments, the protease comprises a Kallikrein (KLK), a kallikrein hK1, a kallikrein hK2, a kallikrein hK3, a kallikrein hK4, a kallikrein hK5, a kallikrein hK6, a kallikrein hK7, a kallikrein hK8, a kallikrein hK9, a kallikrein hK10, a kallikrein hK11, a kallikrein hK12, a kallikrein hK13, a kallikrein hK14, a kallikrein hK15, a Kell blood-group protein, a KHNYN KH and NYN domain containing, a lactotransferrin, a legumain, a leishmanolysin-2, a leucyl aminopeptidase, a leucyl-cystinyl aminopeptidase, a leukotriene A4 hydrolase, a lysosomal carboxypeptidase A, a lysosomal Pro-X C-peptidase, or a combination hereof.

In some embodiments, the protease comprises a membrane metallo-endopeptidase (MME), a macrophage elastase, a macrophage-stimulating protein, a mammalian tolloid-like 1 protein, a mammalian tolloid-like 2 protein, a MAP1D methione aminopeptidase 1D, a marapsin, a marapsin 2, a MASP1/3 (a MBL associated serine protease 3), a MBL associated serine protease 2 (MASP2), a mastin, a matrilysin, a matrilysin-2, a matriptase, a matriptase-2, a matriptase-3, a membrane dipeptidase, a membrane dipeptidase 2, a membrane dipeptidase 3, a membrane-type mosaic Ser-protein, a meprin alpha subunit, a meprin beta subunit, a mesoderm-specific transcript, a mesotrypsin, a methionyl aminopeptidase I, a methionyl aminopeptidase II, a methionyl aminopeptidase II-like, a mitochondrial inner membrane protease 2, a mitochondrial Intermediate peptidase, a mitochondrial Proc. peptidase b-subunit, a mitochondrial proc. protease, a mitochondrial signal peptidase, a matrix metalloproteinase (MMP), a MMP19, a MMP21, a MMP23A, a MMP23B, a MMP27, a MPND, a MT1-MMP, a MT2-MMP, a MT3-MMP, a MT4-MMP, a MT5-MMP, a MT6-MMP, a MYSM1, or a combination hereof.

In some embodiments, the protease comprises a NAALADASE II, a NAALADASE like 2, a NAALADASE like1, a napsin A, a napsin B, a nardilysin, a nasal embryonic LHRH factor, a NEDD4 binding protein 1, a neprilysin, a neprilysin-2, a neurolysin, a neurotrypsin, a neutrophil elastase (ELANE, ELA2), a NLRP1 self-cleaving protein, a nuclear recept. interacting protein 2, a nuclear recept. interacting protein 3, a nucleoporin 98, a NYN domain and retroviral integrase containing, a NY-REN-60, an OMA1, an O-sialoglycoprotein endopeptidase, an O-sialoglycoprotein endopeptidase like 1, an osteoblast serine protease, an OTU domain containing 6B, an OTU domain containing-1, an OTU domain containing-3, an OTU domain containing-5, an OTU domain containing-6A, an otubain-1, an otubain-2, an OTUD2/YOD1, an ovastacin, an oviductin-like/ovochymase-2, an ovochymase-like, or a combination hereof.

In some embodiments, the protease comprises a proteinase 3 (PRTN3), a papain, a PACE4 proprotein convertase, a pancreatic elastase, a pancreatic elastase II (IIA), a pancreatic elastase II form B, a pancreatic endopeptidase E (A), a pancreatic endopeptidase E (B), a pappalysin-1, a pappalysin-2, a paracaspase, a paraplegin, a pepsin A, a pepsin C, a PHEX endopeptidase, a PIDD auto-processing protein unit 1, a PIM1 endopeptidase, a PIM2 endopeptidase, a pitrilysin metalloproteinase 1, a plasma Glu-carboxypeptidase, a plasma kallikrein, a plasma-kallikrein-like 2, a plasma-kallikrein-like 3, a plasma-kallikrein-like 4, a plasmin (plasminogen), a PM20D2 peptidase, a POH1/PSMD14, a polyserase-2, a polyserase-3, a polyserase-I, a Ppnx, a presenilin 1, a presenilin 2, a presenilin homolog 1/SPPL3, a presenilin homolog 2, a presenilin homolog 3/SPP, a presenilin homolog 4/SPPL2B, a presenilin homolog 5, a presenilins-assoc. rhomboid like, a procollagen C-proteinase, a proliferation-association protein 1, a prolyl oligopeptidase, a prolyl oligopeptidase-like, a proprotein convertase 1, a proprotein convertase 2, a proprotein convertase 4, a proprotein convertase 5, a proprotein convertase 7, a proprotein convertase 9 (a proprotein convertase subtilisin/kexin type 9, PCSK9), a prostasin, (a protease, serine, 56), a proteasome alpha 1 subunit, a proteasome alpha 2 subunit, a proteasome alpha 3 subunit, a proteasome alpha 3-like subunit, a proteasome alpha 4 subunit, a proteasome alpha 5 subunit, a proteasome alpha 6 subunit, a proteasome alpha 7 subunit, a proteasome alpha 8 subunit, a proteasome b subunit LMP7-like, a proteasome beta 1 subunit, a proteasome beta 2 subunit, a proteasome beta 3 subunit, a proteasome beta 3-like subunit, a proteasome beta 4 subunit, a proteasome catalytic sub. 1-like, a proteasome catalytic subunit 1, a proteasome catalytic subunit 1i, a proteasome catalytic subunit 2, a proteasome catalytic subunit 2i, a proteasome catalytic subunit 3, a proteasome catalytic subunit 3i, a protein C, a protein C-like, a protein Z, a proteinase 3, a PRPF8, a PSMD7, a pyroglutamyl-peptidase I, a pyroglutamyl-peptidase II, or a combination hereof.

In some embodiments, the protease comprises a reelin, a renin, a retinol binding protein 3, a rhomboid 5 homolog 1, a rhomboid 5 homolog 2, a rhomboid domain containing 1, a rhomboid domain containing 2, a rhomboid, veinlet-like 2, a rhomboid, einlet-like 3, a rhomboid-like protein 1, or a combination hereof.

In some embodiments, the protease comprises a serine protease, a serine protease 3 (PRSS3), a S2P protease, a SAD1, a secernin-1, a secernin-2, a secernin-3, a sentrin (SUMO protease 1), a sentrin (SUMO protease 2), a sentrin (SUMO protease 3), a sentrin (SUMO protease 5), a sentrin (SUMO protease 5-like 1), a sentrin (SUMO protease 6), a sentrin (SUMO protease 7), a sentrin (SUMO protease 8), a sentrin (SUMO protease 9), a sentrin (SUMO protease 11), a sentrin (SUMO protease 12), a sentrin (SUMO protease 13), a sentrin (SUMO protease 14), a sentrin (SUMO protease 15), a sentrin (SUMO protease 16), a sentrin (SUMO protease 17), a sentrin (SUMO protease 18), a sentrin (SUMO protease 19), a separase, a seprase, a serine carboxypeptidase 1, a signalase 18 kDa component, a signalase 21 kDa component, a signalase-like 1, a similar to Arabidopsis Ser-prot., a similar to SPUVE, a site-1 protease, a sonic hedgehog protein, a spinesin, a SprT-like N-terminal domain, a stromelysin 1, a stromelysin 2, a stromelysin 3, a suppressor of Ty 16 homolog, or a combination hereof.

In some embodiments, the protease comprises a taspase, a TBP-associated factor 2, a TESP2, a TESP3, a testase 2, a testis serine protease 2, a testis serine protease 3, a testis serine protease 4, a testis serine protease 5, a testis serine protease 6, a testisin, a testis-specific protein tsp50, a thimet oligopeptidase, a thrombin, a thymus-specific serine peptidase, a TINAG related protein, a TMPRSS11A, a t-plasminogen activator, a TRAF-binding protein domain, a transferrin receptor 2 protein, a transferrin receptor protein, a transmembrane Ser-protease 3, a transmembrane Ser-protease 4, a transthyretin, a TRH-degrading ectoenzyme, a tripeptidyl-peptidase I, a tripeptidyl-peptidase II, a trypsin, a trypsin 10, a trypsin 15, a trypsin C, a trypsin X2, a tryptase, a tryptase alpha/beta 1, a tryptase beta 2, a tryptase delta 1, a tryptase gamma 1, a tryptase homolog 2/EOS, a tryptase homolog 3, a tubulointerstitial nephritis antigen, or a combination hereof.

In some embodiments, the protease comprises a ubiquitin C-term. hydrolase BAP1, a ubiquitin C-terminal hydrolase 1, a ubiquitin C-terminal hydrolase 3, a ubiquitin C-terminal hydrolase 4, a ubiquitin C-terminal hydrolase 5, a ubiquitin specific peptidase like 1, a UCR1, a UCR2, a UDP-N-acetylglucosaminyltransferase subunit, a Ufm-1 specific protease 1, a Ufm-1 specific protease 2, a urokinase (PLAU, uPA) a umbelical vein proteinase, a u-plasminogen activator, a USP1, a USP2, a USP3, a USP4, a USP5, a USP6, a USP7, a USP8, a USP9X, a USP9Y, a USP10, a USP11, a USP12, a USP13, a USP14, a USP15, a USP16, a USP17, a USP17-like, a USP18, a USP19, a USP20, a USP21, a USP22, a USP24, a USP25, a USP26, a USP27, a USP28, a USP29, a USP30, a USP31, a USP34, a USP35, a USP36, a USP37, a USP40, a USP41, a USP42, a USP43, a USP44, a USP45, a USP46, a USP47, a USP48, a USP49, a USP50, a USP51, a USP52, a USP53, a USP54, or a combination hereof.

In some embodiments, the protease comprises a VCP (p97)/p47-interacting protein, a VDU1, a vitellogenic carboxypeptidase-L, a X-Pro dipeptidase, a X-prolyl aminopeptidase 2, a YME1-like 1, a zinc finger CCCH-type containing 12A, a zinc finger CCCH-type containing 12B, a zinc finger CCCH-type containing 12C, a zinc finger CCCH-type containing 12D, a Zinc finger containing ubiquitin peptidase 1, or a combination hereof.

In some embodiments, the protease comprises an A20 (Tumor necrosis factor, alpha-induced protein 3, TNF a-induced protein 3). A20 is a zinc finger protein and a deubiquitinating enzyme. A20 has been shown to inhibit NF-kappa B activation as well as TNF-mediated apoptosis, limit inflammation.

In some embodiments, the protease comprises an Angiotensin-converting enzyme 2 (ACE2). ACE2 is an enzyme attached to the membrane cells located to the membrane of cells located in the intestines, kidney, testis, gallbladder, and heart. ACE2 counters the activity of the related angiotensin-converting enzyme, ACE, by reducing the amount of angiostatin II.

In some embodiments, the protease comprises a cathepsin. The cathepsin may be, but is not limited to, a cathepsin A (CTSA), a cathepsin B (CTSB), a cathepsin C (CTSC), a cathepsin D (CTSD), a cathepsin E (CTSE), a cathepsin H (CTSH), a cathepsin K (CTSK), a cathepsin L (CTSL), a cathepsin S (CTSS), a cathepsin V (CTSV), and a cathepsin Z (CTSZ). Cathepsins are a subset of proteases, many of which become activated in low pH. Cathepsisns comprise serine proteases, cysteine proteases, and aspartyl proteases, among others. Cathepsins have been implicated in cancer, Alzheimer's disease, arthritis, Ebola, pancreatitis, glaucoma, COPD, and other diseases.

In some embodiments, the protease comprises a caspase. The caspase may be, but is not limited to, a caspase 1, a caspase 2, a caspase 3, a caspase 4, a caspase 5, a caspase 6, a caspase 7, a caspase 8, a caspase 9, a caspase 10, a caspase 11, a caspase 12, a caspase 13, and a caspase 14.

In some embodiments, the protease comprises a calpain. The calpain may be, but is not limited to a calpain 1, a calpain 2, a calpain 3, a calpain 4, a calpain 5, a calpain 6, a calpain 7, a calpain 8, a calpain 9, a calpain 10, a calpain 11, a calpain 12, a calpain 13, a calpain 14, and a calpain 15. Caspases are a family of protease enzymes that play essential roles in programmed cell death and cell homeostasis.

In some embodiments, the protease comprises a cysteine protease. Cysteine proteases, also known as thiol proteases, are hydrolase enzymes that degrade proteins. These proteases share a common catalytic mechanism that involves a nucleophilic cysteine thiol in a catalytic triad or dyad. The cysteine protease family comprises Papain (Carica papaya), bromelain (Ananas comosus), cathepsin K (liverwort), calpain (Homo sapiens), aspase-1 (Rattus norvegicus), separase (Saccharomyces cerevisiae), Adenain (human adenovirus type 2), Pyroglutamyl-peptidase I (Bacillus amyloliquefaciens), Sortase A (Staphylococcus aureus), Hepatitis C virus peptidase 2 (hepatitis C virus), Sindbis virus-type nsP2 peptidase (sindbis virus), Dipeptidyl-peptidase VI (Lysinibacillus sphaericus), DeSI-1 peptidase (Mus musculus), TEV protease (tobacco etch virus), Amidophosphoribosyltransferase precursor (Homo sapiens), Gamma-glutamyl hydrolase (Rattus norvegicus), Hedgehog protein (Drosophila melanogaster) and DmpA aminopeptidase (Ochrobactrum anthropi), etc.

In some embodiments, the protease comprises a complement C1r serine protease (Complement component 1r). In some embodiments, the protease comprises a complement C1s serine protease (Complement component 1s). C1r along with C1q and C1s form the C1 complex. C1r has very narrow trypsin-like specificity that is responsible for activation of the C1 complex. C1 activation is a two-step process involving (1) C1r intramolecular autoactivation and (2) C1s cleavage by activated C1r. C1r contains a chymotrypsin-like serine protease domain at its C-terminal, and cleaves a single Arg-Ile bond in C1r and in C1s. Zvi Fishelson, in xPharm: The Comprehensive Pharmacology Reference, 2007.

In some embodiments, the protease comprises a chymotrypsin (chymotrypsins A and B, alpha-chymar ophth, avazyme, chymar, chymotest, enzeon, quimar, quimotrase, alpha-chymar, alpha-chymotrypsin A, alpha-chymotrypsin)). Chymotrypsin is a digestive enzyme component of pancreatic juice acting in the duodenum, where it performs proteolysis, the breakdown of proteins and polypeptides. Chymotrypsin preferentially cleaves peptide amide bonds where the side chain of the amino acid N-terminal to the scissile amide bond is a large hydrophobic amino acid (tyrosine, tryptophan, and phenylalanine).

In some embodiments, the protease comprises a chymase (mast cell protease 1, skeletal muscle protease, skin chymotryptic proteinase, mast cell serine proteinase, skeletal muscle protease). Chymases are a family of serine proteases found in mast cells, basophil granulocytes. Chymases show broad peptidolytic activity and are involved in inflammatory response, hypertension and atherosclerosis.

In some embodiments, the protease comprises a dipeptidyl peptidase (DPP). DPP comprises cathepsin C (DPP1), DPP2, DPP3, DPP4, DPP 6, DPP7, DPP8, DPP9, DPP10.

In some embodiments, the protease comprises a DPP4 (adenosine deaminase complexing protein 2, CD26). DPP4 is expressed on cell surface and is associated with immune regulation, signal transduction, and apoptosis. DPP4 is a serine exopeptidase that cleaves X-proline or X-alanine dipeptides from the N-terminus of polypeptides. DPP-4 is known to cleave a broad range of substrates including growth factors, chemokines, neuropeptides, and vasoactive peptides. DPP4 plays a major role in glucose metabolism, is responsible for the degradation of incretins such as GLP-1, and appears to work as a suppressor in the development of some tumors

In some embodiments, the protease comprises a DPP1 (Cathepsin C, CTSC). DPP1 is a lysosomal exo-cysteine protease belonging to the peptidase C1 family. Cathepsin C appears to be a central coordinator for activation of many serine proteases in immune/inflammatory cells. Cathepsin C catalyzes excision of dipeptides from the N-terminus of protein and peptide substrates,

In some embodiments, the protease comprises a disintegrin and metalloproteinase (ADAM). ADAMs are a family of single-pass transmembrane and secreted metalloendopeptidases. Not all human ADAMs have a functional protease domain. Those ADAMs which are active proteases are classified as sheddases because they cut off or shed extracellular portions of transmembrane proteins.

In some embodiments, the protease comprises an ADAM12 metalloprotease. ADAM12 binds insulin growth factor binding protein-3 (IGFBP-3), appears to be an early Down syndrome marker, and has been implicated in a variety of biological processes involving cell-cell and cell-matrix interactions, including fertilization, muscle development, and neurogenesis.

In some embodiments, the protease comprises a disintegrin and metalloproteinase with thrombospondin motifs (ADAMTS). ADAMTS is a family of multidomain extracellular protease enzymes, comprising ADAMTS1, ADAMTS2, ADAMTS3, ADAMTS4, ADAMTS5 (=ADAMTS11), ADAMTS6, ADAMTS7, ADAMTS8 (or METH-2), ADAMTS9, ADAMTS10, ADAMTS12, ADAMTS13, ADAMTS14, ADAMTS15, ADAMTS16, ADAMTS17, ADAMTS18, ADAMTS19, and ADAMTS20. Known functions of the ADAMTS proteases include processing of procollagens and von Willebrand factor as well as cleavage of aggrecan, versican, brevican and neurocan, making them key remodeling enzymes of the extracellular matrix. They have been demonstrated to have important roles in connective tissue organization, coagulation, inflammation, arthritis, angiogenesis and cell migration.

In some embodiments, the protease comprises an ADAMTS1. ADAMTS1 is a member of the ADAMTS protein family. The expression of ADAMTS1 may be associated with various inflammatory processes, development of cancer cachexia, normal growth, fertility, and organ morphology and function.

In some embodiments, the protease comprises a Factor VII activating protease (FSAP). FSAP is a circulating serine protease with high homology to fibrinolytic enzymes, and may be associated with the regulation of coagulation and fibrinolysis.

In some embodiments, the protease comprises a furin. Furin belongs to the subtilisin-like proprotein convertase family and is a calcium-dependent serine endoprotease. Furin's substrates includes: proparathyroid hormone, transforming growth factor beta 1 precursor, proalbumin, pro-beta-secretase, membrane type-1 matrix metalloproteinase, beta subunit of pro-nerve growth factor and von Willebrand factor.

In some embodiments, the protease comprises a histone deacetylase (HDAC). HDACs are a class of enzymes that remove acetyl groups (O—C—CH3) from an ε-N-acetyl lysine amino acid on a histone, allowing the histones to wrap the DNA more tightly.

In some embodiments, the protease comprises a HTRA1 serine protease. HTRA1 is a secreted enzyme that is proposed to regulate the availability of insulin-like growth factors (IGFs) by cleaving IGF-binding proteins. It has also been suggested to be a regulator of cell growth.

In some embodiments, the protease comprises a granzyme. Granzymes are serine proteases released by cytoplasmic granules within cytotoxic T cells and natural killer (NK) cells. Granzymes induce programmed cell death in the target cell. Granzymes also kill bacteria and inhibit viral replication.

In some embodiments, the protease comprises, a Kallikrein (KLK). Kallikreins are a subgroup of serine proteases. Kallikreins are responsible for the coordination of various physiological functions including blood pressure, semen liquefaction and skin desquamation.

In some embodiments, the protease comprises a matrix metalloproteinase (MMP, matrix metallopeptidases, matrixins). MPPs are calcium-dependent zinc-containing endopeptidases. MMPs have been implicated in cleavage of cell surface receptors, the release of apoptotic ligands, chemokine/cytokine inactivation, cell proliferation and cell migration.

In some embodiments, the protease comprises a membrane metallo-endopeptidase (MME). MME is a zinc-dependent metalloprotease that cleaves peptides at the amino side of hydrophobic residues and inactivates several peptide hormones including glucagon, enkephalins, substance P, neurotensin, oxytocin, and bradykinin. MME is expressed in a wide variety of tissues and is particularly abundant in kidney. MME is also a common acute lymphocytic leukemia antigen.

In some embodiments, the protease comprises a mannose-binding protein-associated serine protease 2 (MASP2, Mannan-binding lectin serine protease 2, MBL associated serine protease 2). MASP2 is involved in the complement system, cleaves complement components C4 and C2 into C4a, C4b, C2a, and C2b.

In some embodiments, the protease comprises a mannose-binding protein-associated serine protease 3 (MBL associated serine protease 3, MASP3). MASP3 originates from the MASP1 gene through differential splicing, it circulates in high serum concentrations predominantly in complex with Ficolin-3 and regulates Ficolin-3 mediated complement activation.

In some embodiments, the protease comprises a neutrophil elastase (ELANE, ELA2). ELANE is a serine proteinase secreted by neutrophils and microphages during inflammation and destroys bacteria and host tissue.

In some embodiments, the protease comprises a proteinase 3 (PRTN3). PRTN3 is a serine protease enzyme expressed mainly in neutrophil granulocytes and contributes to the proteolytic generation of antimicrobial peptides.

In some embodiments, the protease comprises a plasmin (a.k.a. plasminogen). Plasmin is a proteolytic enzyme derived from an inert plasma precursor known as plasminogen. It is present in blood that degrades many blood plasma proteins, including fibrin clots. In human, plasmin is encoded by PLG gene.

In some embodiments, the protease comprises a pepsin. Pepsin is an endopeptidase that cleaves proteins into smaller peptides. It is an aspartic protease, using a catalytic aspartate in its active site.

In some embodiments, the protease comprises a presenilin-1 (PS-1). PS-1 is a presenilin protein that is one of the four core proteins in the gamma secretase complex, which is considered to play an important role in generation of amyloid beta from amyloid precursor protein.

In some embodiments, the protease comprises a proprotein convertase subtilisin/kexin type 9 (PCSK9). PCSK9 is a member of the peptidase S8 family.

In some embodiments, the protease comprises a serine protease. Serine protease cleaves peptide bonds in proteins, in which serine serves as the nucleophilic amino acid at the enzyme's active site. Serine protease includes many subfamilies.

In some embodiments, the protease comprises a tryptase. Tryptase is a the most abundant secretory granule-derived serine proteinase contained in mast cells and has been used as aa marker for mast cell activation. It is released from mask cells when they are activated as part of a normal immune response as well as in allergic responses.

In some embodiments, the protease comprises, a trypsin. Trypsin is a serine protease from the PA clan superfamily, found in the digestive system. Trypsin cuts peptide chains mainly at the carboxyl side of the amino acids lysine or arginine.

In some embodiments, the protease comprises a urokinase (PLAU, uPA). Urokinase is a serine protease present in humans and other animals. It is present in human urine, blood and in the extracellular matrix of many tissues. It is involved in degradation of the extracellular matrix and possibly tumor cell migration and proliferation. Urokinase is a 411-residue protein, consisting of three domains: the serine protease domain, the kringle domain, and the EGF-like domain. Urokinase is synthesized as a zymogen form (prourokinase or single-chain urokinase), and is activated by proteolytic cleavage between Lys158 and Ile159. The two resulting chains are kept together by a disulfide bond.

Described herein are agents to be detected including but are not limited to a oxidoreductase, a transferase, a hydrolase, a lyase, a isomerase, a ligase, a protease, a hydrolase, an esterase, a β-glycosidase, a phospholipase and a phosphodiesterase, peroxidase, lipase, amylase a nucleophilic reagent, a reducing reagent, a electrophilic/acidic reagent, an organometallic/metal catalyst, an oxidizing reagent, a hydroxyl ion, a thiols nucleophile, a nitrogen nucleophile, a sodium dithionite and a sodium periodate.

As described herein, the activity detection of some agents does not rely on cleavage. For example, some oxidoreductases, transferases, hydrolases, lyases, isomerases, and ligases lead to the substrate linker modification and release or formation of a reporter molecule that can be detected. As a way of illustration, a certain oxidation processes can modify an inactive fluorophore and render it fluorescent/detectable without the need of a substrate linker or binding events (for non-covalent processes) can change magnetic/fluorescent physical-chemical properties of certain reporters and render them detectable.

### Disease and Condition

The method described herein comprise determining a disease or condition of the subject. In some aspects, the disease or condition comprises a liver disease, a cancer, a metabolic disease, a fibrotic disease, an organ transplant rejection, an infectious disease, an allergic disease, an autoimmunity, Alzheimer's or a chronic inflammation. In some embodiments, the liver disease may be a non-alcoholic steatohepatitis (NASH), a non-alcoholic fatty liver disease (NAFLD), a toxin mediated liver injury (drug/medication, alcohol, environmental), a viral hepatitis (HAV, HBV, HCV, HDV, HEV, other virus infecting the liver), an autoimmune hepatitis, a primary biliary cholangitis, a primary sclerosing cholangitis, a fulminant hepatitis, a cirrhosis of the liver, a hepatocellular carcinoma (HCC), a cholangiocarcinoma, an acute or chronic rejection of a transplanted liver, an inherited liver disease (e.g. Wilson disease, hemochromatosis, or alpha-1 antitrypsin) or a combination thereof.

In some embodiments, the cancer comprises adenoid cystic carcinoma, adrenal gland tumors, amyloidosis, anal cancer, appendix cancer, astrocytoma, ataxia-telangiectasia, Beckwith-Wiedemann syndrome, bile duct cancer (cholangiocarcinoma), Birt-Hogg-Dubé Syndrome, bladder cancer, bone cancer (sarcoma of the bone), brain stem glioma, brain tumors, breast cancer, Carney complex, central nervous system tumors, cervical cancer, colorectal cancer, Cowden Syndrome, craniopharyngioma, Desmoid tumors, desmoplastic infantile ganglioglioma, ependymoma, esophageal cancer, Ewing sarcoma, eye cancer, eyelid cancer, familial adenomatous polyposis, familial GIST, familial malignant melanoma, familial pancreatic cancer, gallbladder cancer, gastrointestinal stromal tumors (GIST), germ cell tumors, gestational trophoblastic disease, head and neck cancer, breast and ovarian cancer, diffuse gastric cancer, leiomyosarcoma and renal cell cancer, mixed polyposis syndrome, papillary renal carcinoma, juvenile polyposis syndrome, kidney cancer, lacrimal gland tumors, laryngeal and hypopharyngeal cancer, leukemia, myeloid leukemia, lymphoblastic leukemia, eosinophilic leukemia, Li-Fraumeni syndrome, liver cancer, lung cancer, Hodgkin lung cancer, non-Hodgkin lung cancer, Lynch syndrome, mastocytosis, medulloblastoma, melanoma, meningioma, mesothelioma, multiple endocrine neoplasia, multiple myeloma, myelodysplastic syndrome, nasal cavity and paranasal sinus cancer, nasopharyngeal cancer, neuroblastoma, neuroendocrine tumors, neurofibromatosis, nevoid basal cell carcinoma syndrome, oral and oropharyngeal cancer, osteosarcoma, ovarian cancer, fallopian tube cancer, peritoneal cancer, pancreatic cancer, parathyroid cancer, penile cancer, Peutz-Jeghers syndrome, phenochromocytoma, paraganglioma, pituitary gland tumors, pleuropulmonary blastoma, prostate cancer, retinoblastoma, rhabdomyosarcoma, salivary gland cancer, Kaposi sarcoma, soft tissue sarcoma, sarcoma, non-melanoma skin cancer, small bowel cancer, stomach cancer, testicular cancer, thymoma and thymic carcinoma, thyroid cancer, tuberous sclerosis complex, uterine cancer, vaginal cancer, von Hippel-Lindau syndrome, vulvar cancer, Waldenstrom macroglobulinemia, Werner syndrome, Wilms tumors, or xeroderma pigmentosum.

In some embodiments, the disease may be NASH. Non-alcoholic steatohepatitis, also called NASH, is a more active inflammatory form of non-alcoholic fatty liver disease (NAFLD). NAFLD is caused by buildup of fat in the liver. When this buildup causes inflammation and damage, it is known as NASH, which can lead to scarring of the liver. There are often no outward signs or symptoms associated with NASH, although the most common symptoms are fatigue or mild pain in the upper right abdomen. NASH may lead to cirrhosis of the liver, causing one or more of the following symptoms as the condition progresses: bleeding easily, bruising easily, itchy skin, jaundice, abdominal fluid accumulation, loss of appetite, nausea, leg swelling, confusion, drowsiness, slurred speech, or spider-like blood vessels.

NASH is most common in patients who are overweight or obese; other risk factors include diabetes, high cholesterol, high triglycerides, poor diet, metabolic syndrome, polycystic ovary syndrome, sleep apnea, and hyperthyroidism.

NAFLD encompasses the entire spectrum of fatty liver disease in individuals without significant alcohol consumption, ranging from fatty liver to steatohepatitis to cirrhosis. Non-alcoholic fatty liver is the presence of >5% hepatic steatosis without evidence of hepatocellular injury in the form of ballooning of the hepatocytes or evidence of fibrosis. The risk of progression to cirrhosis and liver failure is considered minimal. NASH is the presence of >5% hepatic steatosis with inflammation and hepatocyte injury (ballooning) with or without fibrosis. This can progress to cirrhosis, liver failure, and rarely liver cancer. NASH cirrhosis is presence of cirrhosis with current or previous histological evidence of steatosis or steatohepatitis.

NAFLD activity score (NAS) is an unweighted composite of steatosis, lobular inflammation, and ballooning scores. NAS is a useful tool to measure changes in liver histology in patients with NAFLD in clinical trials. Fibrosis is scored separately and can be classified as F1 through F4; specifically, stage 1 is zone 3 (perivenular), perisinusoidal, or periportal fibrosis; stage 2 is both zone 3 and periportal fibrosis; stage 3 is bridging fibrosis with nodularity; and stage 4 is cirrhosis.

TABLE 3
The histological scoring system for nonalcoholic fatty liver disease:
components of NAFLD activity score (NAS) and fibrosis staging.

| | | | |
|---|---|---|---|
| Item | Score | Extent | Definition and Comment |

NAS Components (see scoring interpretation)

| Steatosis | 0 | <5% | | Refers to amount of surface |
|---|---|---|---|---|
| | 1 | 5-33% | | area involved by steatosis |
| | 2 | >33-66% | | as evaluated on low to |
| | 3 | >66% | | medium power examination. |
| Lobular | 0 | No foci | | Acidophil bodies are not |
| Inflammation | 1 | <2 | foci/200x | included in this assessment, |
| | 2 | 2-4 | foci/200x | nor is portal inflammation |
| | 3 | >4 | foci/200x | |

| | | | |
|---|---|---|---|
| Hepatocyte | 0 | None | |
| Ballooning | 1 | Few ballooned | “Few” means rare but definite |
| | | cells | ballooned hepatocytes as |
| | | | well as cases that are |
| | | | diagnostically borderline |
| | 2 | Many | Most cases with prominent |
| | | cells/prominent | ballooning also had Mallory”s |
| | | ballooning | hyalin, but Mallory”s hyaline |
| | | | is not scoredseparately for |
| | | | the NAS |

Fibrosis Stage (Evalutaed separately from NAS)

| | | | |
|---|---|---|---|
| Fibrosis | 0 | None | |
| | 1 | Perisinusoidal | |
| | | or periportal | |
| | 1A | Mild, zone 3, | “delicate”fibrosis |
| | | perisinusoidal | |
| | 1B | Moderate, | “dense”fibrosis |
| | | zone 3, | |
| | | perisinusoidal | |
| | 1C | Portal/periportal | This category is included to |
| | | | accommodate cases with |
| | | | portal and/or peri portal |
| | | | fibrosis without |
| | | | accompanying pericellular/ |
| | | | perisinusoidal fibrosis |
| | 2 | Perisinusoidal and | |
| | | portal/periportal | |
| | 3 | Bridging fibrosis | |
| | 4 | Cirrhosis | |

Scoring interpretation: Total NAS score represents the sum of scores for steatosis, lobular inflammation, and ballooning, and ranges from 0-8. Diagnosis of NASH (or, alternatively, fatty liver not diagnostic of NASH) should be made first, then NAS is used to grade activity. In the reference study, NAS scores of 0-2 occurred in cases largely considered not diagnostic of NASH, scores of 3-4 were evenly divided among those considered not diagnostic, borderline, or positive for NASH. Scores of 5-8 occurred in cases that were largely considered diagnostic of NASH

In some embodiments, the disease comprises NAFLD. Nonalcoholic fatty liver disease (NAFLD) is an umbrella term for a range of liver conditions affecting people who drink little to no alcohol. As the name implies, the main characteristic of NAFLD is too much fat stored in liver cells. There are often no outward signs or symptoms associated with NAFLD, although the most common symptoms are fatigue or mild pain in the upper right abdomen.

In some embodiments, the disease comprises fulminant hepatitis. Fulminant hepatitis, or fulminant hepatic failure, is defined as a clinical syndrome of severe liver function impairment, which causes hepatic coma and the decrease in synthesizing capacity of liver. Then they rapidly develop severe, often life-threatening liver failure. This can happen within hours, days, or sometimes weeks. Symptoms of severe liver failure include confusion, extreme irritability, altered consciousness, blood clotting defects, and buildup of fluid in the abdominal cavity and multiorgan system failure.

In some embodiments, the disease comprises a hepatocellular carcinoma (HCC). HCC is the most common type of primary liver cancer. HCC occurs most often in people with chronic liver diseases leading to advanced fibrosis or cirrhosis. The most common liver diseases associated with HCC are viral hepatitis B or C, alcohol related liver disease and NASH.

In some embodiments, the disease comprises a primary biliary cholangitis (PBC). Primary biliary cholangitis, also referred to as primary biliary cirrhosis, is a chronic disease in which the bile ducts in the liver are slowly destroyed. Bile is a fluid made in the liver. Chronic inflammation in the liver can lead to bile duct damage, irreversible scarring of liver tissue (cirrhosis) and eventually, liver failure. PBC is considered an autoimmune disease, which means the body's immune system is mistakenly attacking healthy cells and tissue. Researchers think a combination of genetic and environmental factors triggers the disease. It usually develops slowly. At this time, there is no cure for primary biliary cholangitis, but medication can slow liver damage, especially if treatment begins early.

In some embodiments, the liver disease comprises a toxin mediated liver injury (e.g., from drug/medication, alcohol, environmental). Toxin mediated liver injury is an inflammation of liver in reaction to certain substances, such as alcohol, chemicals, drugs/medication, environmental factors or nutritional supplements. The liver normally removes and breaks down most drugs and chemicals from the bloodstream, which creates byproducts that can damage the liver. Although the liver has a great capacity for regeneration, constant exposure to toxic substances can cause serious, sometimes irreversible harm.

In some embodiments, the liver disease comprises a viral hepatitis (HAV, HBV, HCV, HDV, HEV, other virus infecting the liver). Viral hepatitis is a liver inflammation due to a viral infection. It may present in acute form as a recent infection with relatively rapid onset, or in chronic form. The most common causes of viral hepatitis are the five unrelated hepatotropic viruses hepatitis A, B, C, D, and E. Other viruses can also cause liver inflammation, including cytomegalovirus, Epstein-Barr virus, and yellow fever. There also have been scores of recorded cases of viral hepatitis caused by herpes simplex virus. Viral hepatitis is either transmitted through contaminated food or water (A, E) or via blood and body fluids (B, C). Hepatitis A and hepatitis B can be prevented by vaccination. Effective treatments for hepatitis C are available but costly.

In some embodiments, the liver disease comprises an autoimmune hepatitis. Autoimmune hepatitis is liver inflammation that occurs when the immune system attacks liver cells. The exact cause of autoimmune hepatitis is unclear, but genetic and environmental factors appear to interact over time in triggering the disease. Untreated autoimmune hepatitis can lead to scarring of the liver (cirrhosis) and eventually to liver failure. When diagnosed and treated early, autoimmune hepatitis often can be controlled with drugs that suppress the immune system. A liver transplant may be an option when autoimmune hepatitis does not respond to drug treatments or in cases of advanced liver disease. There are two main forms of autoimmune hepatitis: (1) Type 1 autoimmune hepatitis. Type I autoimmune hepatitis is the most common type and can occur at any age. About half the people with type 1 autoimmune hepatitis have other autoimmune disorders, such as celiac disease, rheumatoid arthritis or ulcerative colitis; (2) Type 2 autoimmune hepatitis. Although adults can develop type 2 autoimmune hepatitis, it is most common in children and young people. Other autoimmune diseases may accompany type 2 autoimmune hepatitis.

In some embodiments, the liver disease comprises a primary sclerosing cholangitis. Primary sclerosing cholangitis is a disease of the bile ducts. In primary sclerosing cholangitis, inflammation causes scars within the bile ducts. These scars make the ducts hard and narrow and gradually cause serious liver damage. A majority of people with primary sclerosing cholangitis also have inflammatory bowel disease, such as ulcerative colitis or Crohn's disease. In most cases of primary sclerosing cholangitis, the disease progresses slowly. It can eventually lead to liver failure, repeated infections, and tumors of the bile duct or liver.

In some embodiments, the liver disease comprises a cirrhosis of the liver. Cirrhosis is a late stage of scarring (fibrosis) of the liver caused by many forms of liver diseases and conditions, such as hepatitis and chronic alcoholism. In the process of liver self-repair, scar tissue forms. As cirrhosis progresses, more and more scar tissue forms, making it difficult for the liver to function (decompensated cirrhosis).

In some embodiments, the liver disease comprises a cholangiocarcinoma. Cholangiocarcinoma (bile duct cancer) is a type of cancer that forms in the bile ducts. Risk factors for cholangiocarcinoma include primary sclerosing cholangitis (an inflammatory disease of the bile ducts), ulcerative colitis, cirrhosis, hepatitis C, hepatitis B, infection with certain liver flukes, and some congenital liver malformations. Cholangiocarcinoma can be categorized based on the location of the cancer occurs in the bile ducts: intrahepatic cholangiocarcinoma, hilar cholangiocarcinoma, distal cholangiocarcinoma. Cholangiocarcinoma is often diagnosed when it is advanced, making successful treatment difficult to achieve.

In some embodiments, the liver disease comprises an inherited liver disease (e.g., Wilson's disease, hemochromatosis, or alpha-1 antitrypsin, etc.) Inherited liver diseases are genetic disorders that can cause severe liver disease and other health problems. Wilson's disease is a rare inherited disorder that causes copper to accumulate in your liver, brain and other vital organs. Hemochromatosis is a disease in which deposits of iron collect in the liver and other organs. The primary form of hemochromatosis is one of the most common inherited diseases in the U.S. The alpha-1 antitrypsin protein is synthesized mainly in the liver by hepatocytes, secreted into the blood stream, and acts as an inhibitor of neutrophil elastase released primarily in the lung during inflammation. Alpha-1 antitrypsin deficiency is caused when alpha-1 antitrypsin protein is either lacking or exists in lower than normal levels in the blood.

In some embodiments, the disease may be an organ transplant rejection. Transplant rejection occurs when transplanted tissue is rejected by the recipient's immune system, which destroys the transplanted tissue. Transplant rejection can be lessened by determining the molecular similitude between donor and recipient and by use of immunosuppressant drugs after transplant.

In some embodiments, the disease comprises an infectious disease, Infectious diseases are disorders caused by organisms-such as bacteria, viruses, fungi or parasites. Bacteria are one-cell organisms responsible for illnesses such as streptococcal upper respiratory infection, urinary tract infections and tuberculosis. Viruses cause a multitude of diseases ranging from the common cold to AIDS. Many skin diseases, such as ringworm and athlete's foot, are caused by fungi. Other types of fungi can infect the lungs or nervous system. Malaria is caused by a tiny parasite that is transmitted by a mosquito bite. Other parasites may be transmitted to humans from animal feces. In some embodiments, the infectious disease is COVID-19.

In some embodiments, the disease comprises an allergic disease. Allergic diseases are caused by allergen-induced unfavorable immune responses initiating various symptoms in different organs, which often cannot be completely controlled by modern medicine. The immunologic basis of allergic diseases is observed in two phases: sensitization and development of memory T and B cell responses, and IgE production and effector functions, which are related to eosinophils, innate lymphoid cells, dendritic cell subsets, epithelial cells and tissue inflammation/injury, epithelial barrier, tissue remodeling and chronicity in asthma, atopic dermatitis (AD) and allergic rhinitis (AR). Different disease phenotypes and endotypes may become apparent with different dominant molecular mechanisms, related biomarkers and responses to biologic therapy. Multiple mechanistic factors are complexly involved in the pathogenesis of allergic inflammations.

In some embodiments, the disease comprises an autoimmune disease/autoimmunity. An autoimmune disease is a condition in which the immune system mistakenly attacks one's own body. Normally, the immune system can tell the difference between foreign cells and one's own cells. In an autoimmune disease, the immune system mistakes part of the body, like the joints or skin, as foreign. It releases proteins called autoantibodies that attack healthy cells. Some autoimmune diseases target only one organ. Type 1 diabetes damages the pancreas. Other diseases, like systemic lupus erythematosus (SLE), affect many different organ systems. In some embodiments, the autoimmune disease may be Rheumatoid arthritis, Crohn's disease, Multiple sclerosis (MS) or psoriatic arthritis (PsA).

In some embodiments, the disease comprises a chronic inflammation. Chronic inflammation is also referred to as slow, long-term inflammation lasting for prolonged periods of several months to years. Generally, the extent and effects of chronic inflammation vary with the cause of the injury and the ability of the body to repair and overcome the damage. Most of the features of acute inflammation continue as the inflammation becomes chronic, including the expansion of blood vessels (vasodilation), increase in blood flow, capillary permeability and migration of neutrophils into the infected tissue through the capillary wall (diapedesis). However, the composition of the white blood cells changes soon and the macrophages and lymphocytes begin to replace short-lived neutrophils. Thus the hallmarks of chronic inflammation are the infiltration of the primary inflammatory cells such as macrophages, lymphocytes, and plasma cells in the tissue site, producing inflammatory cytokines, growth factors, enzymes and hence contributing to the progression of tissue damage and secondary repair including fibrosis and granuloma formation, etc.

In some embodiments, the disease comprises a fibrotic disease. Fibrotic disease is defined by the overgrowth, hardening, and/or scarring of various tissues and is attributed to excess deposition of extracellular matrix components including collagen. Fibrosis is the end result of chronic inflammatory reactions induced by a variety of stimuli including persistent infections, autoimmune reactions, allergic responses, chemical insults, radiation, and tissue injury. The fibrotic disorders include but are not limited to systemic fibrotic diseases such as systemic sclerosis (SSc), sclerodermatous graft vs. host disease, idiopathic pulmonary fibrosis (IPF), nephrogenic systemic fibrosis, and organ-specific disorders including radiation-induced fibrosis and cardiac, pulmonary, liver, and kidney fibrosis.

In some embodiments, the disease comprises a metabolic disease. A metabolic disorder/disease occurs when abnormal chemical reactions in the body disrupt metabolism. When this happens, one might have too much of some substances or too little of other ones that an individual needs to stay healthy. There are different groups of disorders. Some affect the breakdown of amino acids, carbohydrates, or lipids. Another group, mitochondrial diseases, affects the parts of the cells that produce the energy. one can develop a metabolic disorder when some organs, such as the liver or pancreas, become diseased or do not function normally. Diabetes is an example.

In some embodiments, the disease comprises Alzheimer's. Alzheimer's is a type of dementia that affects memory, thinking and behavior. Symptoms eventually grow severe enough to interfere with daily tasks. Alzheimer's changes typically begin in the part of the brain that affects learning. As Alzheimer's advances through the brain, it leads to increasingly severe symptoms, including disorientation, mood and behavior changes; deepening confusion about events, time and place; unfounded suspicions about family, friends and professional caregivers; more serious memory loss and behavior changes; and difficulty speaking, swallowing and walking.

### EXAMPLES

These prophetic examples are provided for illustrative purposes only and not to limit the scope of the claims provided herein. It will be appreciated that variations in proportions and alternatives in elements of the components shown will be apparent to those skilled in the art and are within the scope of the embodiments presented herein.

### Example 1. Conjugation Chemistry and Preparation of Synthetic Molecule

Example embodiments demonstrate chemistries for conjugating a linker to a synthetic polymer. In some example embodiments, the synthetic polymer is a dendrimer. In some example embodiments, the dendrimer is a polyamidoamine dendrimer with an ethyldiamine core. A linker is conjugated, via a first end of the linker, to a dendrimer by preparing a reaction mixture including a molar excess of [200 moles]. Reaction is performed with 200 molar excess of linker to dendrimer in 0.1M NaHCO₃ for 2 hrs at room temperature. To keep DMF concentration low, volume is adjusted to maintain a final DMF concentration of 0.15%. Reaction is performed at 10 mg/mL of dendrimer with 200 molar excess of functionalized linker.

Once the linker is conjugated to the dendrimer, a compound comprising a peptide sequence conjugates to a second end of the linker upon preparing a reaction mixture comprising 0.08 mg of the dendrimer conjugated to the linker, at a concentration of 10 mg/mL, and the compound comprising the peptide sequence, at a concentration of 1 mM. The addition is performed in conditions tailored specifically to the peptide. In this example, we used neutral pH PBS for the reaction with 0.08 mg of dendrimer functionalized with linker and 10 molar equivalents of peptide per addition into a final volume of 1500 uL. Each 10 equivalent addition step is performed for 1 hr under rotation at room temperature protected from light. A total of up to 9 additions, with 10 molar equivalents per addition is sufficient to achieve complete conjugation. Following full conjugation after 90 equivalents, the final polymer functionalized with peptides is filtered using a 10,000 Da MWCO filter, using 4×4000 rpm spins for 10 minutes each, diluting 10× each time with a final concentration step. Pre and post analysis is done using HPLC for size exclusion analysis.

### Example 2. Quantifying Molar Amount of IEPD Conjugated to Synthetic Polymer

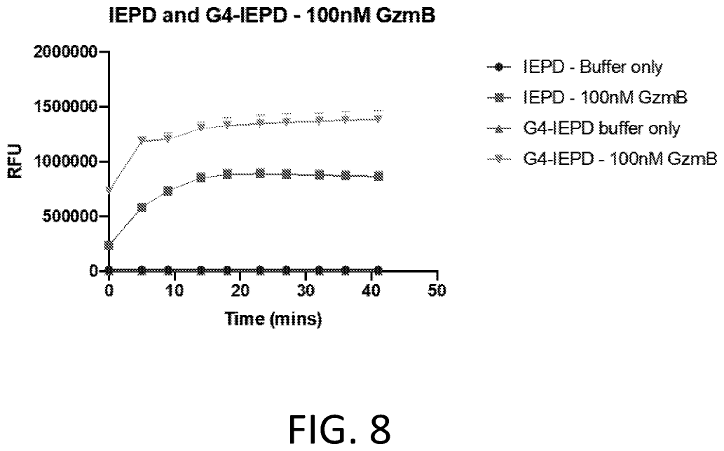

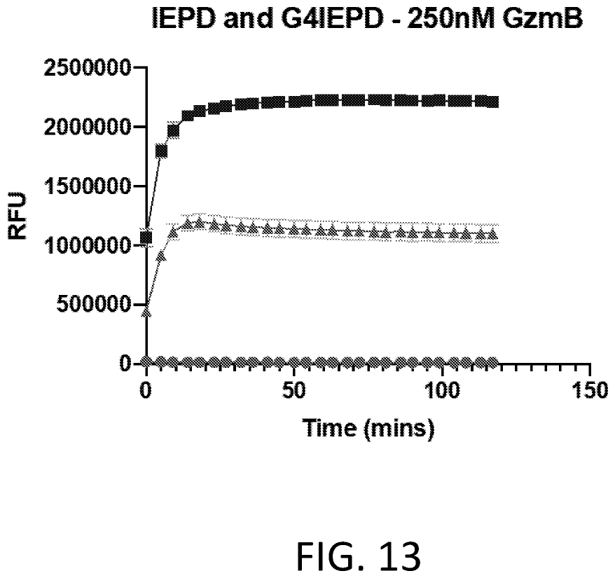

To quantify the amount of molar peptide conjugated to the dendrimer, we run a calibration curve on HPSEC to determine a typical peak area for a known concentration of linear peptide using the fluorescence detector on the HPLC. We then quantify peak area of the conjugate against known concentration peaks of linear peptide only and determine a rough concentration (FIG. 7). This concentration is then verified using an enzymatic cleavage assay which determines concentration as a function of total RFU (Raw Fluorescence Units) when compared to a linear peptide (as shown in FIGS. 8 and 13).

### Example 3. Michaelis-Menten Kinetics—G4 IEPD

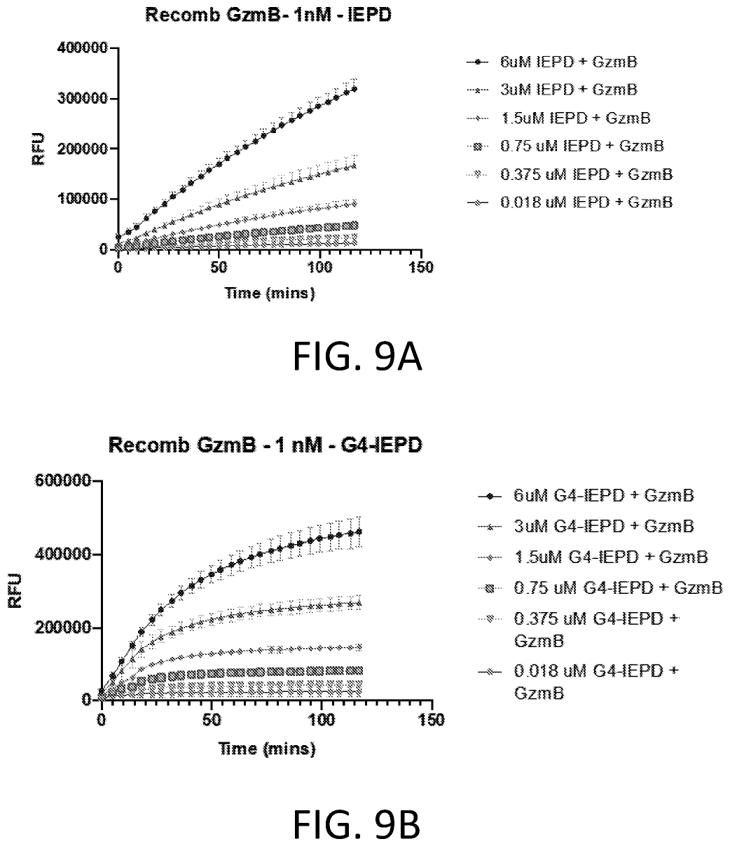

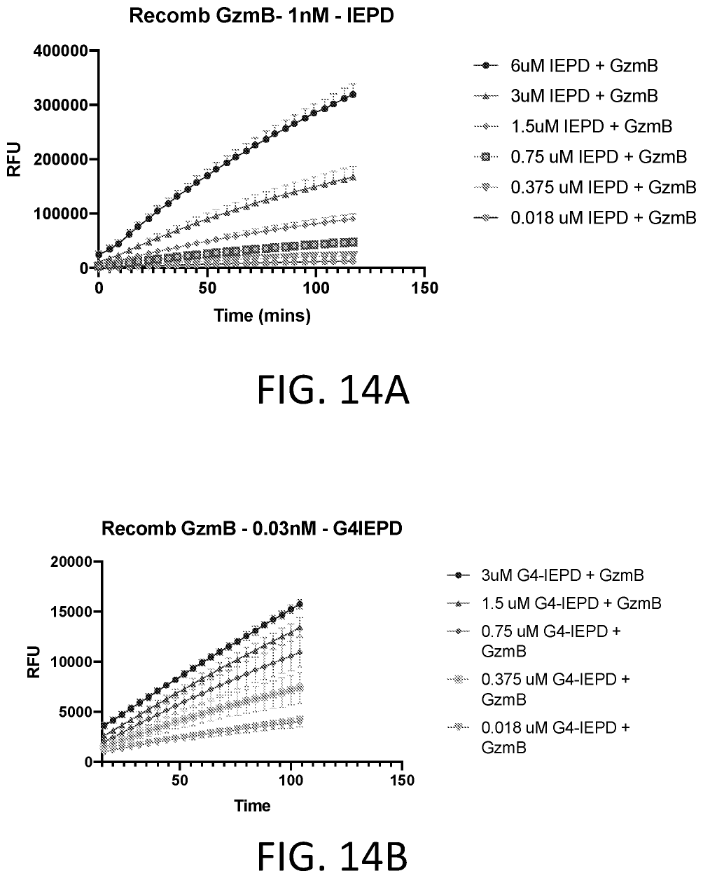

To determine Michaelis Menten kinetics, as shown in FIGS. 9 and 14, both the linear peptide and polymer conjugated peptide are run in a titration series from high to low micromolar concentration of molecule using an enzyme with known cleavage of the peptide sequence at a predetermined concentration, typically in the nanomolar range. We perform these reactions on a 384-well plate using a SpectraMax plate reader to monitor fluorescence changes from the sensors over time. With this cleavage data, we are able to extract Michaelis Menten kinetics using a Lineweaver Burke plot using the known substrate concentration reciprocal and known velocity of the reaction. From this Lineweaver Burke plot we can extract the information necessary to calculate kcat, km, and Vmax. These constants allow us to compare the kinetics of both the linear peptide and the peptide conjugated dendrimer construct.

### Example 4. Limit of Detection—Linear IEPD Vs. G4-IEPD in a Buffer-Based System

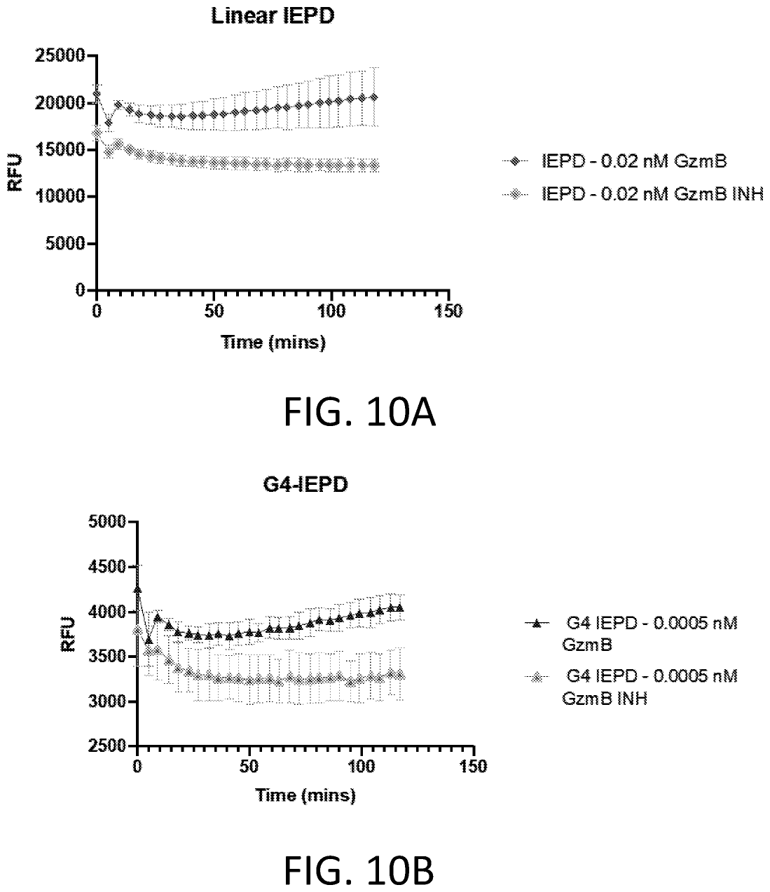

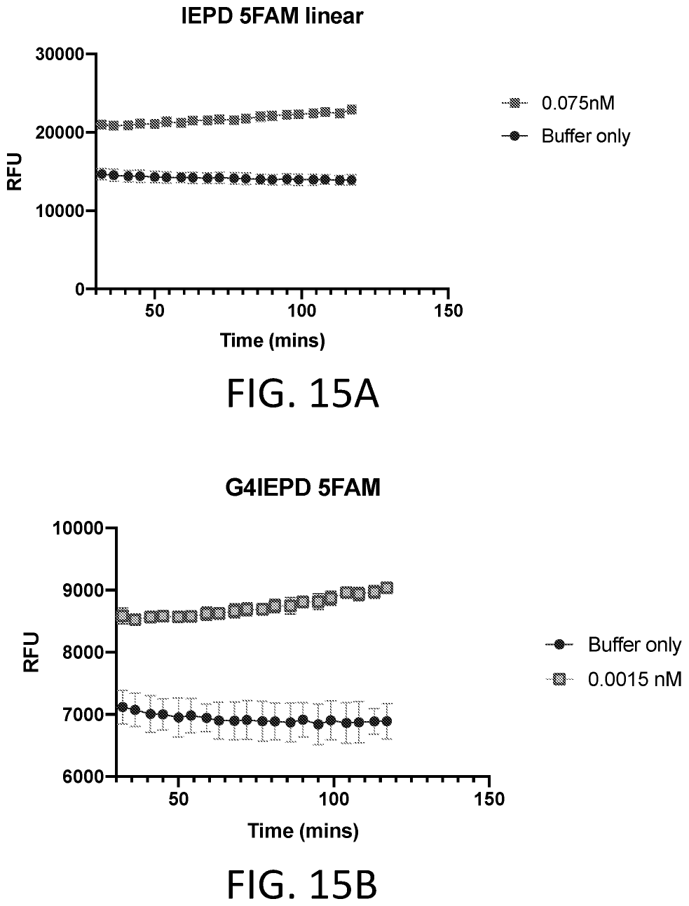

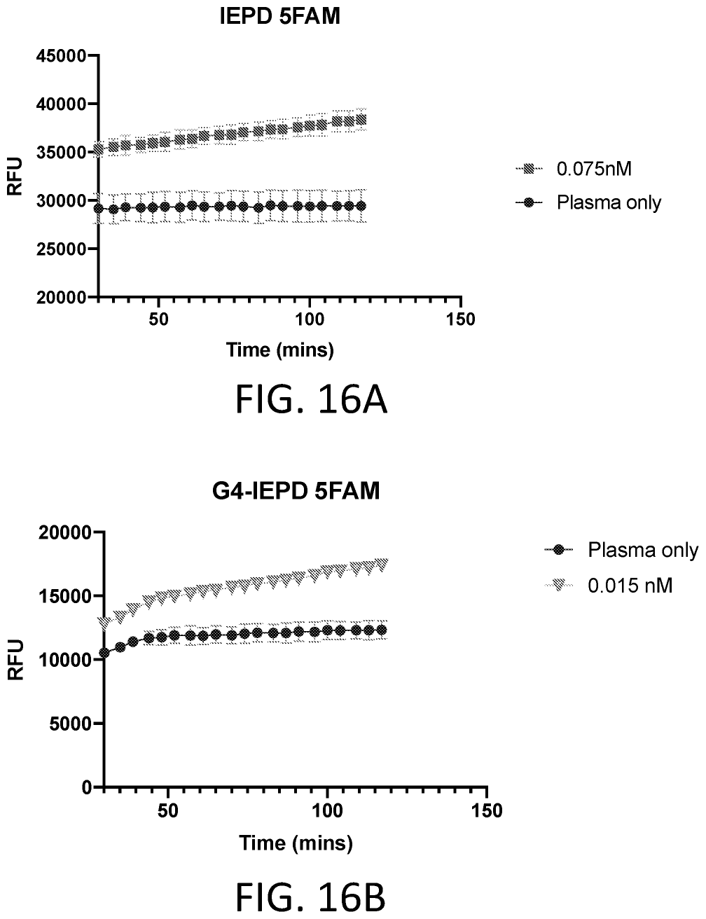

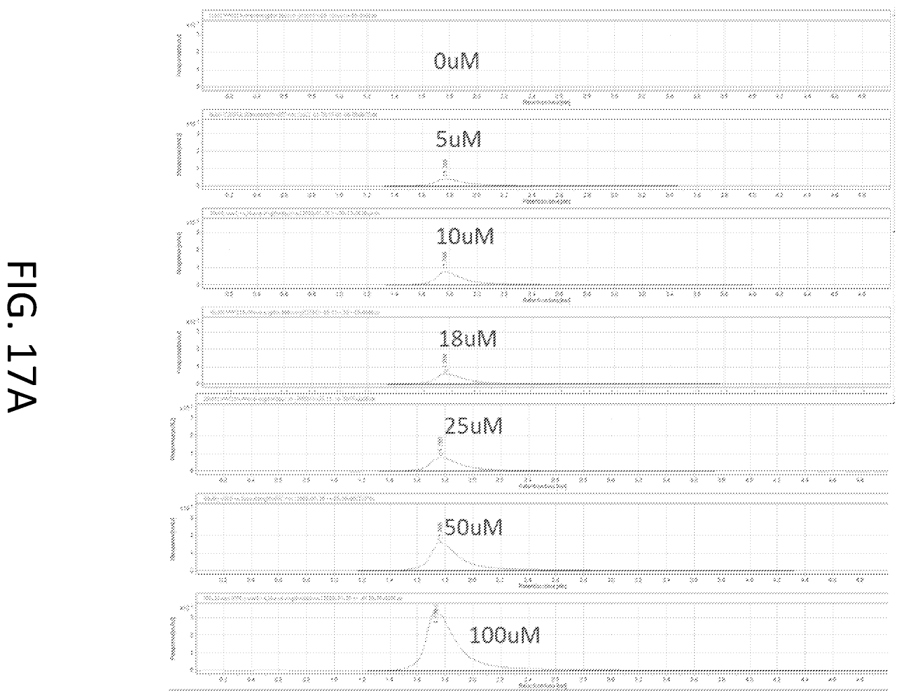

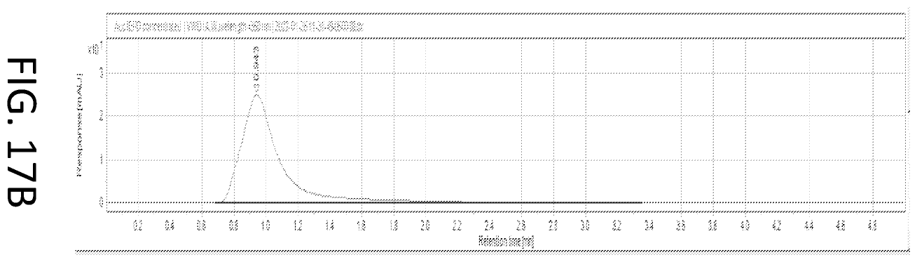

To determine the lower limit of detection using the linear and dendrimer conjugates in a buffer based system, we take a known recombinant human enzyme and spike it into a specific buffer at a predetermined dilution series and determine the lowest amount of spike-in enzyme that is detectable, using each peptide construct at 6 uM and sub nanomolar to picomolar concentrations of enzyme, as shown in FIGS. 10 and 15. There is an observed increase in sensitivity using the dendrimer peptide construct when compared to the detection limits of the linear peptide.

### Example 5. Alternative Biofluids Outside of Plasma-Improved Sensitivity and Lower

### Background

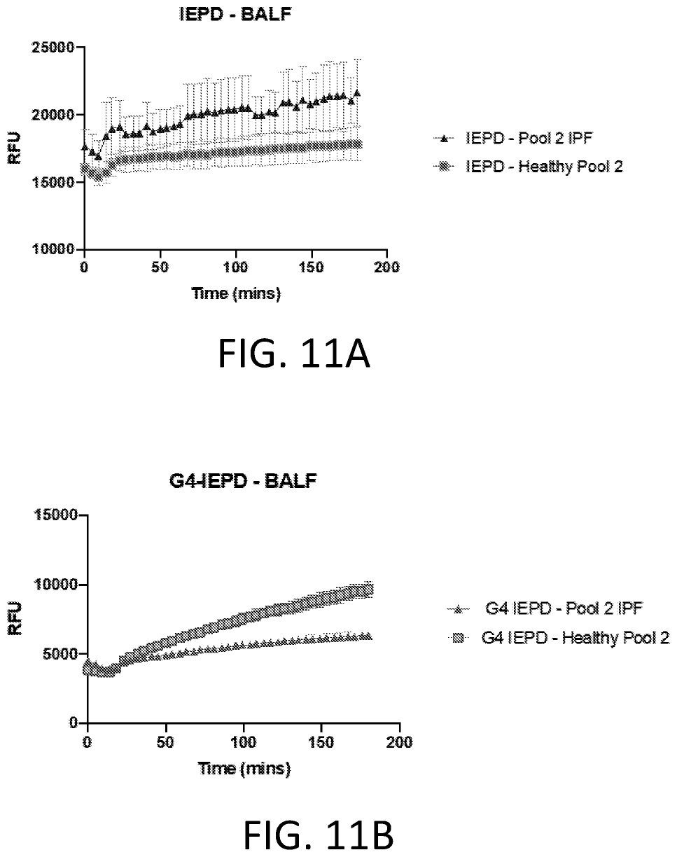

As shown in FIG. 11, we are able to detect differential activity using a biofluid of interest that has implications for lung applications. Pools of each biofluid from selected patients are used, both disease and healthy controls, and diluted in the desired buffer to compare the activity of the linear peptide and dendrimer conjugated peptide at 6 uM concentration and monitor the kinetics of the reaction over time using a fluorescence-based output measured by the SpectraMax plate reader. From this data, we are able to differentiate disease and healthy samples only when using the dendrimer peptide construct, with the linear peptide demonstrating little to no differential activity.

### Example 6. COVID Detection Swabs

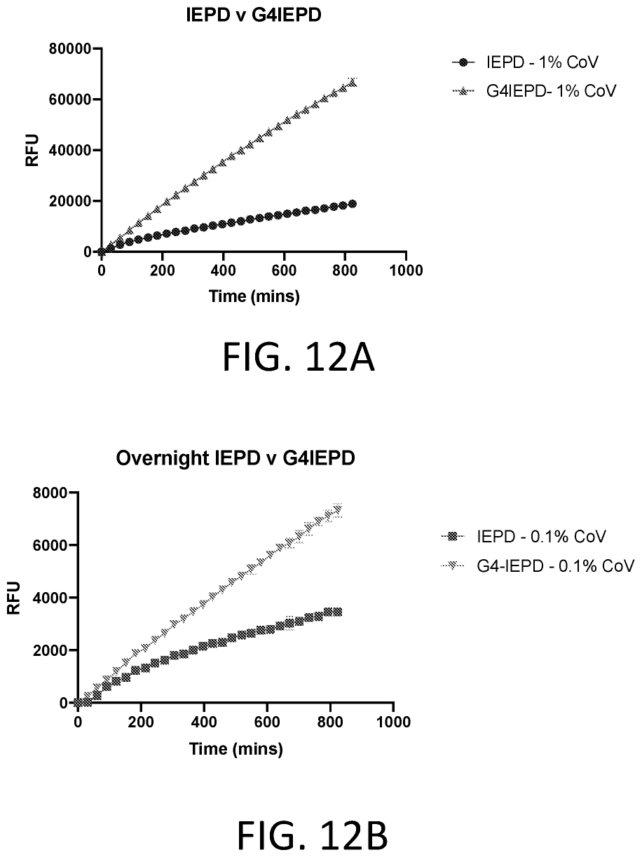

To improve detection of immune activity in COVID swabs in saline media, we applied the dendrimer construct in comparison to the linear peptide to determine COVID associated enzyme activity from the swabs. The swab saline media was pooled from COVID patient samples and tested with the linear peptide and dendrimer construct peptide at 6 uM and we were able to dilute samples down to 0.1% and still detect differential activity between the linear construct and the dendrimer construct using a fluorescence based read output on the SpectraMax plate reader. This demonstrates a higher sensitivity detection method using a swab saline matrix diluted to 0.1%, as shown in FIG. 12.

What is claimed is:

1. A synthetic molecule comprising:
(a) a synthetic polymer comprising a core, a plurality of branch points, and a plurality of endpoints;
(b) a plurality of linkers, wherein a linker of the plurality of linkers comprises (i) a linker sequence, (ii) a first end, and (iii) a second end, wherein the first end is coupled to an endpoint of the plurality of endpoints; and
(c) a plurality of peptide sequences, wherein a peptide sequence of the plurality of peptide sequences is coupled to the second end of the linker,
wherein the synthetic molecule is configured to react with an enzyme present in a sample obtained from a subject.

2. The synthetic molecule of claim 1, wherein the second end comprises a reactive handle.

3. The synthetic molecule of claim 1, wherein each of the plurality of peptide sequences comprise a sequence that is at least 60% homologous to other sequences in the plurality of peptide sequences.

4. The synthetic molecule of claim 1, wherein the plurality of peptide sequences comprises different peptide sequences.

5. The synthetic molecule of claim 1, wherein the plurality of peptide sequences comprises a combination of a first set of peptide sequences and a second set of peptide sequences, wherein each peptide sequence of the first set of peptide sequences is similar, and the second set of peptide sequences comprises different peptide sequences.

6. The synthetic molecule of claim 1, wherein each of the plurality of linkers comprises a spacer coupled to an organic molecule.

7. The synthetic molecule of claim 6, wherein the spacer comprises a PEG sequence.

8. The synthetic molecule of claim 6, wherein the organic molecule comprises an imide, a tetrazine, a cyclooctyne, an azide, an alkyne, a phosphine, a norbornene, a thiol, an alkene, an aldehyde, a hydroxylamine, a diene, a dienophile, a hydroxysuccinimide, or an amine.

9. The synthetic molecule of claim 8, wherein the imide comprises formula (I):

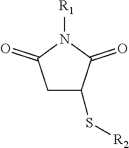

10. The synthetic molecule of claim 1, wherein the core comprises an amine core.

11. The synthetic molecule of claim 10, wherein the amine core comprises an ethylenediamine core or a polyamidoamine core.

12. The synthetic molecule of claim 1, wherein each of the plurality of branch points, the plurality of endpoints, or a combination thereof, are configured to exhibit different chemical properties from one another.

13. The synthetic molecule of claim 1, wherein the reaction with the enzyme indicates an enzyme activity.

14. The synthetic molecule of claim 13, wherein the enzyme activity comprises a disease-related enzyme activity, a baseline enzyme activity, or a combination thereof.

15. The method of claim 13, wherein the enzyme comprises a protease, and wherein the enzyme activity comprises a protease activity.

16. The synthetic molecule of claim 15, wherein the protease activity indicates a presence of a pathogen, and wherein the presence of the pathogen is associated with a disease.

17. The synthetic molecule of claim 1, further comprising a probe.

18. The synthetic molecule of claim 17, wherein the probe is selected from Table 1.

19. The synthetic molecule of claim 17, wherein the synthetic molecule comprises an IEPD dendrimer, and wherein the probe comprises a probe 9 molecule, a probe 102 molecule, a probe 379 molecule, or a combination thereof.

20. The synthetic molecule of claim 1, further comprising a plurality of probes.

21. The synthetic molecule of claim 20, wherein the plurality of probes are selected from Table 1.

22. The synthetic molecule of claim 20, wherein the IEPD dendrimer comprises a G4-IEPD dendrimer, a G5-IEPD dendrimer, a G6-IEPD dendrimer, a G7-IEPD dendrimer, a G8-IEPD dendrimer, a G9-IPED dendrimer, or a G10-IEPD dendrimer.

23. The synthetic molecule of claim 15, wherein the protease is selected from the group consisting of an A20 (TNFa-induced protein 3), an abhydrolase domain containing 4, an abhydrolase domain containing 12, an abhydrolase domain containing 12B, an abhydrolase domain containing 13, an acrosin, an acylaminoacyl-peptidase, a disintegrin and metalloproteinase (ADAM), an ADAM1a, an ADAM2 (Fertilin-b), an ADAM3B, an ADAM4, an ADAM4B, an ADAM5, an ADAM6, an ADAM7, an ADAM8, an ADAM9, an ADAM10, an ADAM11, an ADAM12 metalloprotease, an ADAM15, an ADAM17, an ADAM18, an ADAM19, an ADAM20, an ADAM21, an ADAM22, an ADAM23, an ADAM28, an ADAM29, an ADAM30, an ADAM32, an ADAM33, a disintegrin and metalloproteinase with thrombospondin motifs (ADAMTS), an ADAMTS1, an ADAMTS2, an ADAMTS3, an ADAMTS4, an ADAMTS5/11, an ADAMTS6, an ADAMTS7, an ADAMTS8, an ADAMTS9, an ADAMTS10, an ADAMTS12, an ADAMTS13, an ADAMTS14, an ADAMTS15, an ADAMTS16, an ADAMTS17, an ADAMTS18, an ADAMTS19, an ADAMTS20, an adipocyte-enhancer binding protein 1, an Afg3-like protein 1, an Afg3-like protein 2, an airway-trypsin-like protease, an aminoacylase, an aminopeptidase A, an aminopeptidase B, an aminopeptidase B-like 1, an aminopeptidase MAMS/L-RAP, an aminopeptidase N, an aminopeptidase O, an aminopeptidase P homologue, an aminopeptidase P1, an aminopeptidase PILS, an aminopeptidase Q, an aminopeptidase-like 1, an AMSH/STAMBP, an AMSH-LP/STAMBPL1, an angiotensin-converting enzyme 1 (ACE1), an angiotensin-converting enzyme 2 (ACE2), an angiotensin-converting enzyme 3 (ACE3), an anionic trypsin (II), an apolipoprotein (a), an archaemetzincin-1, an archaemetzincin-2, an aspartoacylase, an aspartoacylase-3, an aspartyl aminopeptidase, an ataxin-3, an ataxin-3 like, an ATP/GTP binding protein 1, an ATP/GTP binding protein-like 2, an ATP/GTP binding protein-like 3, an ATP/GTP binding protein-like 4, an ATP/GTP binding protein-like 5, an ATP23 peptidase, an autophagin-1, an autophagin-2, an autophagin-3, an autophagin-4, an azurocidin, a beta lactamase, a beta-secretase 1, a beta-secretase 2, a bleomycin hydrolase, a brain serine proteinase 2, a BRCC36 (BRCA2-containing complex, sub 3), a calpain, a calpain 1, a calpain 2, a calpain 3, a calpain 4, a calpain 5, a calpain 6, a calpain 7, a calpain 7-like, a calpain 8, a calpain 9, a calpain 10, a calpain 11, a calpain 12, a calpain 13, a calpain 14, a calpain 15 (Solh protein), a cysteine protease, a carboxypeptidase A1, a carboxypeptidase A2, a carboxypeptidase A3, a carboxypeptidase A4, a carboxypeptidase A5, a carboxypeptidase A6, a carboxypeptidase B, a carboxypeptidase D, a carboxypeptidase E, a carboxypeptidase M, a carboxypeptidase N, a carboxypeptidase O, a carboxypeptidase U, a carboxypeptidase X1, a carboxypeptidase X2, a carboxypeptidase Z, a carnosine dipeptidase 1, a carnosine dipeptidase 2, a caspase recruitment domain family, member 8, a caspase, a caspase-1, a caspase-2, a caspase-3, a caspase-4/11, a caspase-5, a caspase-6, a caspase-7, a caspase-8, a caspase-9, a caspase-10, a caspase-12, a caspase-14, a caspase-14-like, a casper/FLIP, a cathepsin, a cathepsin A (CTSA), a cathepsin B (CTSB), a cathepsin C (CTSC), a cathepsin D (CTSD), a cathepsin E (CTSE), a cathepsin F, a cathepsin G, a cathepsin H (CTSH), a cathepsin K (CTSK), a cathepsin L (CTSL), a cathepsin L2, a cathepsin O, a cathepsin S (CTSS), a cathepsin V (CTSV), a cathepsin W, a cathepsin Z (CTSZ), a cationic trypsin, a cezanne/OTU domain containing 7B, a cezanne-2, a CGI-58, a chymase, a chymopasin, a chymosin, a chymotrypsin B, a chymotrypsin C, a coagulation factor IXa, a coagulation factor VIIa, a coagulation factor Xa, a coagulation factor XIa, a coagulation factor XIIa, a collagenase 1, a collagenase 2, a collagenase 3, a complement protease C1r serine protease, a complement protease C1s serine protease, a complement C1r-homolog, a complement component 2, a complement component C1ra, a complement component C1sa, a complement factor B, a complement factor D, a complement factor D-like, a complement factor I, a COPS6, a corin, a CSN5 (JAB1), a cylindromatosis protein, a cytosol alanyl aminopep.-like 1, a cytosol alanyl aminopeptidase, a DDI-related protease, a DECYSIN, a Der1-like domain family, member 1, a Der1-like domain family, member 2, a Der1-like domain family, member 3, a DESC1 protease, a desert hedgehog protein, a desumoylating isopeptidase 1, a desumoylating isopeptidase 2, a dihydroorotase, a dihydropyrimidinase, a dihydropyrimidinase-related protein 1, a dihydropyrimidinase-related protein 2, a dihydropyrimidinase-related protein 3, a dihydropyrimidinase-related protein 4, a dihydropyrimidinase-related protein 5, a DINE peptidase, a dipeptidyl peptidase (DPP), a dipeptidyl peptidase (DPP1), a dipeptidyl-peptidase 4 (DPP4), a dipeptidyl-peptidase 6 (DPP6), a dipeptidyl-peptidase 8 (DPP8), a dipeptidyl-peptidase 9 (DPP9), a dipeptidyl-peptidase II, a dipeptidyl-peptidase III, a dipeptidyl-peptidase 10 (DPP10), a DJ-1, a DNA-damage inducible protein, a DNA-damage inducible protein 2, a DUB-1, a DUB-2, a DUB2a, a DUB2a-like, a DUB2a-like2, a DUB6, or a combination thereof.

24. The synthetic molecule of claim 15, wherein the protease is selected from the group consisting of a T cell protease, a complement protease, a fibrosis protease, and an inflammation-related protease.

25. The synthetic molecule of claim 1, wherein the synthetic molecule further comprises a carrier.

26. The synthetic molecule of claim 25, wherein the carrier comprises a native, labeled or synthetic protein, a synthetic chemical polymer of precisely known chemical composition or with a distribution around a mean molecular weight, an oligonucleotide, a phosphorodiamidate morpholino oligomer (PMO), a foldamer, a lipid, a lipid micelle, a nanoparticle, a solid support made of polystyrene, polypropylene or any other type of plastic, or any combination thereof.

27. The synthetic molecule of claim 1, wherein the linker comprises a peptide, a carbohydrate, a nucleic acid, a lipid, an ester, a glycoside, a phospholipid, a phosphodiester, a nucleophile/base sensitive linker, a reduction sensitive linker, an electrophile/acid sensitive linker, a metal cleavable linker, an oxidation sensitive linker or a combination thereof.

28. The synthetic molecule of claim 1, wherein the enzyme present in the sample is configured to bind to a binding site on the synthetic molecule, and wherein the synthetic molecule is present in the sample at a concentration of 0.01 nM-0.1M.

29. The synthetic molecule of claim 1, wherein the enzyme is present in the sample at a concentration of between approximately 0.01 nM-1.0 nM.

30. The synthetic molecule of claim 1, wherein the plurality of peptide sequences is configured to have an increased affinity to the enzyme in comparison to a linear peptide sequence not linked to the synthetic molecule.

31. The synthetic molecule of claim 1, wherein the plurality of peptide sequences comprises approximately 1-50 peptides, 50-100 peptides, or 100-150 peptides.

32. The synthetic molecule of claim 31, wherein the plurality of peptide sequences are different, similar, or a combination thereof.

33. The synthetic molecule of claim 1, wherein the second end comprises a tunable sequence.

34. The synthetic molecule of claim 1, wherein the synthetic molecule is configured react with a paper strip application.

35. The synthetic molecule of claim 1, wherein the synthetic polymer comprises a dendrimer, a multivalent synthetic macromolecule, a nanoparticle scaffold, a polymeric scaffold, a foldamer, a branched peptide, or a synthetic composite nanoparticle.

36. The synthetic molecule of claim 1, wherein the peptide sequence comprises a reporter, a peptide sequence spacer, a reactive handle, and a binding site for the enzyme.

37. The synthetic molecule of claim 36, wherein the reporter comprises a fluorescent molecule.

38. The synthetic molecule of claim 37, wherein the fluorescent molecule comprises a FRET peptide.

39. A method comprising:
(a) contacting a body fluid sample obtained from a subject with a synthetic molecule, wherein the synthetic molecule comprises:
(i) a dendrimer
(ii) a plurality of linkers coupled to the dendrimer, and
(iii) at least one peptide sequence coupled to the plurality of linkers, wherein the at least one peptide sequence comprises a reporter and a binding site for an enzyme present in the body fluid sample, wherein the synthetic molecule reacts with the enzyme from the body fluid, causing the reporter to generate a detectable signal, and
(b) detecting the detectable signal.

40. The method of claim 39, wherein a linker of the plurality of linkers comprises a spacer coupled to an organic molecule.

41. The method of claim 40, wherein the spacer comprises a PEG sequence.

42. The method of claim 41, wherein the PEG sequence comprises a PEG2 sequence.

43. The synthetic molecule of claim 40, wherein the organic molecule comprises an imide, a tetrazine, a cyclooctyne, an azide, an alkyne, or a phosphine.

44. The method of claim 43, wherein the imide comprises formula (I):

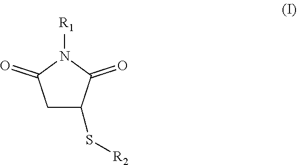

45. The method of claim 39, wherein the synthetic molecule further comprises an amine core.

46. The method of claim 45, wherein the amine core comprises an ethylenediamine core or a polyamidoamine core.

47. The method of claim 39, wherein the synthetic molecule is configured to detect activity of the enzyme.

48. The method of claim 39, wherein the detectable signal is generated by an activity of the enzyme.

49. The method of claim 47 or claim 48, wherein the enzyme activity comprises a disease-related enzyme activity, a baseline enzyme activity, or a combination thereof.

50. The method of claim 47, wherein the synthetic molecule is configured to detect the enzyme activity in a proximal biofluid.

51. The method of claim 48, wherein the detectable signal is generated by an enzyme activity in a proximal biofluid.

52. The method of claim 50 or 51, wherein the enzyme activity indicates a presence of a pathogen, wherein the pathogen is associated with a disease.

53. The method of claim 39, wherein the reporter comprises a fluorescent molecule.

54. The method of claim 53, wherein the fluorescent molecule comprises a FRET peptide.

55. The method of claim 39, further comprising a probe.

56. The method of claim 55, wherein the probe is selected from Table 1.

57. The method of claim 55, wherein the synthetic molecule further comprises an IEPD dendrimer, and the probe comprises a probe 9 molecule, a probe 102 molecule, a probe 379 molecule, or a combination thereof.

58. The method of claim 39, further comprising a plurality of probes.

59. The method of claim 58, wherein the plurality of probes are selected from Table 1.

60. The method of claim 39, wherein the dendrimer comprises an IPED dendrimer, and wherein the probe comprises a probe 102 molecule or a probe 379 molecule.

61. The method of claim 60, wherein the IEPD dendrimer comprises a G4-IEPD dendrimer, a G5-IEPD dendrimer, a G6-IEPD dendrimer, a G7-IEPD dendrimer, a G8-IEPD dendrimer, a G9-IPED dendrimer, or a G10-IEPD dendrimer.

62. The method of claim 39, wherein the enzyme comprises a protease.

63. The method of claim 62, wherein the protease is selected from the group consisting of an A20 (TNFa-induced protein 3), an abhydrolase domain containing 4, an abhydrolase domain containing 12, an abhydrolase domain containing 12B, an abhydrolase domain containing 13, an acrosin, an acylaminoacyl-peptidase, a disintegrin and metalloproteinase (ADAM), an ADAMla, an ADAM2 (Fertilin-b), an ADAM3B, an ADAM4, an ADAM4B, an ADAM5, an ADAM6, an ADAM7, an ADAM8, an ADAM9, an ADAM10, an ADAM11, an ADAM12 metalloprotease, an ADAM15, an ADAM17, an ADAM18, an ADAM19, an ADAM20, an ADAM21, an ADAM22, an ADAM23, an ADAM28, an ADAM29, an ADAM30, an ADAM32, an ADAM33, a disintegrin and metalloproteinase with thrombospondin motifs (ADAMTS), an ADAMTS1, an ADAMTS2, an ADAMTS3, an ADAMTS4, an ADAMTS5/11, an ADAMTS6, an ADAMTS7, an ADAMTS8, an ADAMTS9, an ADAMTS10, an ADAMTS12, an ADAMTS13, an ADAMTS14, an ADAMTS15, an ADAMTS16, an ADAMTS17, an ADAMTS18, an ADAMTS19, an ADAMTS20, an adipocyte-enhancer binding protein 1, an Afg3-like protein 1, an Afg3-like protein 2, an airway-trypsin-like protease, an aminoacylase, an aminopeptidase A, an aminopeptidase B, an aminopeptidase B-like 1, an aminopeptidase MAMS/L-RAP, an aminopeptidase N, an aminopeptidase O, an aminopeptidase P homologue, an aminopeptidase P1, an aminopeptidase PILS, an aminopeptidase Q, an aminopeptidase-like 1, an AMSH/STAMBP, an AMSH-LP/STAMBPL1, an angiotensin-converting enzyme 1 (ACE1), an angiotensin-converting enzyme 2 (ACE2), an angiotensin-converting enzyme 3 (ACE3), an anionic trypsin (II), an apolipoprotein (a), an archaemetzincin-1, an archaemetzincin-2, an aspartoacylase, an aspartoacylase-3, an aspartyl aminopeptidase, an ataxin-3, an ataxin-3 like, an ATP/GTP binding protein 1, an ATP/GTP binding protein-like 2, an ATP/GTP binding protein-like 3, an ATP/GTP binding protein-like 4, an ATP/GTP binding protein-like 5, an ATP23 peptidase, an autophagin-1, an autophagin-2, an autophagin-3, an autophagin-4, an azurocidin, a beta lactamase, a beta-secretase 1, a beta-secretase 2, a bleomycin hydrolase, a brain serine proteinase 2, a BRCC36 (BRCA2-containing complex, sub 3), a calpain, a calpain 1, a calpain 2, a calpain 3, a calpain 4, a calpain 5, a calpain 6, a calpain 7, a calpain 7-like, a calpain 8, a calpain 9, a calpain 10, a calpain 11, a calpain 12, a calpain 13, a calpain 14, a calpain 15 (Solh protein), a cysteine protease, a carboxypeptidase A1, a carboxypeptidase A2, a carboxypeptidase A3, a carboxypeptidase A4, a carboxypeptidase A5, a carboxypeptidase A6, a carboxypeptidase B, a carboxypeptidase D, a carboxypeptidase E, a carboxypeptidase M, a carboxypeptidase N, a carboxypeptidase O, a carboxypeptidase U, a carboxypeptidase X1, a carboxypeptidase X2, a carboxypeptidase Z, a carnosine dipeptidase 1, a carnosine dipeptidase 2, a caspase recruitment domain family, member 8, a caspase, a caspase-1, a caspase-2, a caspase-3, a caspase-4/11, a caspase-5, a caspase-6, a caspase-7, a caspase-8, a caspase-9, a caspase-10, a caspase-12, a caspase-14, a caspase-14-like, a casper/FLIP, a cathepsin, a cathepsin A (CTSA), a cathepsin B (CTSB), a cathepsin C (CTSC), a cathepsin D (CTSD), a cathepsin E (CTSE), a cathepsin F, a cathepsin G, a cathepsin H (CTSH), a cathepsin K (CTSK), a cathepsin L (CTSL), a cathepsin L2, a cathepsin O, a cathepsin S (CTSS), a cathepsin V (CTSV), a cathepsin W, a cathepsin Z (CTSZ), a cationic trypsin, a cezanne/OTU domain containing 7B, a cezanne-2, a CGI-58, a chymase, a chymopasin, a chymosin, a chymotrypsin B, a chymotrypsin C, a coagulation factor IXa, a coagulation factor VIIa, a coagulation factor Xa, a coagulation factor XIa, a coagulation factor XIIa, a collagenase 1, a collagenase 2, a collagenase 3, a complement protease C1r serine protease, a complement protease C1s serine protease, a complement Cr-homolog, a complement component 2, a complement component C1ra, a complement component C1sa, a complement factor B, a complement factor D, a complement factor D-like, a complement factor I, a COPS6, a corin, a CSN5 (JAB1), a cylindromatosis protein, a cytosol alanyl aminopep.-like 1, a cytosol alanyl aminopeptidase, a DDI-related protease, a DECYSIN, a Der1-like domain family, member 1, a Der1-like domain family, member 2, a Der1-like domain family, member 3, a DESC1 protease, a desert hedgehog protein, a desumoylating isopeptidase 1, a desumoylating isopeptidase 2, a dihydroorotase, a dihydropyrimidinase, a dihydropyrimidinase-related protein 1, a dihydropyrimidinase-related protein 2, a dihydropyrimidinase-related protein 3, a dihydropyrimidinase-related protein 4, a dihydropyrimidinase-related protein 5, a DINE peptidase, a dipeptidyl peptidase (DPP), a dipeptidyl peptidase (DPP1), a dipeptidyl-peptidase 4 (DPP4), a dipeptidyl-peptidase 6 (DPP6), a dipeptidyl-peptidase 8 (DPP8), a dipeptidyl-peptidase 9 (DPP9), a dipeptidyl-peptidase II, a dipeptidyl-peptidase III, a dipeptidyl-peptidase 10 (DPP10), a DJ-1, a DNA-damage inducible protein, a DNA-damage inducible protein 2, a DUB-1, a DUB-2, a DUB2a, a DUB2a-like, a DUB2a-like2, a DUB6, or a combination thereof.

64. The method of claim 62, wherein the protease is selected from the group consisting of a T-cell protease, a complement protease, a fibrosis protease, and an inflammation-related protease.

65. The method of claim 39, wherein the synthetic molecule further comprises a carrier.

66. The method of claim 65, wherein the carrier comprises a native, labeled or synthetic protein, a synthetic chemical polymer of precisely known chemical composition or with a distribution around a mean molecular weight, an oligonucleotide, a phosphorodiamidate morpholino oligomer (PMO), a foldamer, a lipid, a lipid micelle, a nanoparticle, a solid support comprising polystyrene, polypropylene or any other type of plastic compound, or any combination thereof.

67. The method of claim 39, wherein the plurality of linkers comprises a peptide, a carbohydrate, a nucleic acid, a lipid, an ester, a glycoside, a phospholipid, a phosphodiester, a nucleophile/base sensitive linker, a reduction sensitive linker, an electrophile/acid sensitive linker, a metal cleavable linker, an oxidation sensitive linker, or a combination thereof.

68. The method of claim 39, wherein the synthetic molecule is present in the body fluid sample at a concentration of approximately 0.01 nM-0.1M.

69. The method of claim 39, wherein the enzyme is present in the body fluid sample at a concentration of approximately 0.01 nM-1.0 nM.

70. The method of claim 39, wherein the detectable signal is generated when the enzyme is present in the body fluid sample at a concentration of approximately 0.01 nM to approximately 1.0 nM.

71. The method of claim 39, wherein the peptide sequence is configured to have an increased affinity to an enzyme compared to a linear peptide sequence that is not coupled to the dendrimer.

72. The method of any one of claims 39-71, wherein the synthetic molecule further comprising a plurality of peptide sequences.

73. The method of claim 72, wherein the plurality of peptide sequences comprises approximately 1-50 peptides, 50-100 peptides, or 100-150 peptides.

74. The method of claim 72, wherein the plurality of peptide sequences are different than one another, similar to each other, or a combination thereof.

75. The method of claim 39, wherein the at least one peptide sequence comprises a tunable sequence.

76. The method of claim 39, wherein the synthetic molecule is configured to react with a paper strip application.

77. The method of claim 39, further comprising detecting a rate of generation or an amount of the detectable signal.

78. A method of synthesizing a molecule, comprising
(a) providing linker components comprising an organic molecule and an inert spacer, thereby producing a linker
(b) providing a synthetic polymer comprising a core, a plurality of branch points, a plurality of end points, and a free amino group,
(c) providing a peptide with a free thiol group,
wherein the linker couples to the synthetic polymer via the plurality of endpoints, and
wherein the organic molecule reacts with the free thiol group, thereby covalently binding the peptide to the linker.

79. The method of claim 78, wherein the organic molecule comprises an imide, a maleimide, a tetrazine, a cyclooctyne, an azide, an alkyne, or a phosphine.

80. The method of claim 79, wherein the imide comprises formula (I):

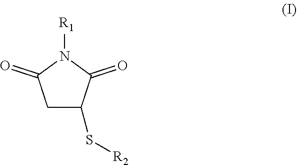

81. The method of claim 78, wherein the organic molecule comprises an N-hydroxysuccinimide (NHS), a maleimide, or a combination thereof.

82. The method of claim 78, wherein the inert spacer comprises a PEG sequence.

83. The method of claim 78, wherein the synthetic molecule comprises an IPED dendrimer and a probe 102 molecule or a probe 379 molecule.

84. The method of claim 83, wherein the IEPD dendrimer comprises a G4-IEPD dendrimer, a G5-IEPD dendrimer, a G6-IEPD dendrimer, a G7-IEPD dendrimer, a G8-IEPD dendrimer, a G9-IPED dendrimer, or a G10-IEPD dendrimer.

85. The method of claim 78, wherein the core comprises an ethylenediamine core or a polyamidoamine core.

86. The method of claim 78, wherein the peptide comprises a sequence having a binding site for an enzyme.

87. The method of claim 86, wherein the enzyme comprises a protease.

88. The method of claim 87, wherein the protease is selected from the group consisting of an A20 (TNFa-induced protein 3), an abhydrolase domain containing 4, an abhydrolase domain containing 12, an abhydrolase domain containing 12B, an abhydrolase domain containing 13, an acrosin, an acylaminoacyl-peptidase, a disintegrin and metalloproteinase (ADAM), an ADAMla, an ADAM2 (Fertilin-b), an ADAM3B, an ADAM4, an ADAM4B, an ADAM5, an ADAM6, an ADAM7, an ADAM8, an ADAM9, an ADAM10, an ADAM11, an ADAM12 metalloprotease, an ADAM15, an ADAM17, an ADAM18, an ADAM19, an ADAM20, an ADAM21, an ADAM22, an ADAM23, an ADAM28, an ADAM29, an ADAM30, an ADAM32, an ADAM33, a disintegrin and metalloproteinase with thrombospondin motifs (ADAMTS), an ADAMTS1, an ADAMTS2, an ADAMTS3, an ADAMTS4, an ADAMTS5/11, an ADAMTS6, an ADAMTS7, an ADAMTS8, an ADAMTS9, an ADAMTS10, an ADAMTS12, an ADAMTS13, an ADAMTS14, an ADAMTS15, an ADAMTS16, an ADAMTS17, an ADAMTS18, an ADAMTS19, an ADAMTS20, an adipocyte-enhaner binding protein 1, an Afg3-like protein 1, an Afg3-like protein 2, an airway-trypsin-like protease, an aminoacylase, an aminopeptidase A, an aminopeptidase B, an aminopeptidase B-like 1, an aminopeptidase MAMS/L-RAP, an aminopeptidase N, an aminopeptidase O, an aminopeptidase P homologue, an aminopeptidase P1, an aminopeptidase PILS, an aminopeptidase Q, an aminopeptidase-like 1, an AMSH/STAMBP, an AMSH-LP/STAMBPL1, an angiotensin-converting enzyme 1 (ACE1), an angiotensin-converting enzyme 2 (ACE2), an angiotensin-converting enzyme 3 (ACE3), an anionic trypsin (II), an apolipoprotein (a), an archaemetzincin-1, an archaemetzincin-2, an aspartoacylase, an aspartoacylase-3, an aspartyl aminopeptidase, an ataxin-3, an ataxin-3 like, an ATP/GTP binding protein 1, an ATP/GTP binding protein-like 2, an ATP/GTP binding protein-like 3, an ATP/GTP binding protein-like 4, an ATP/GTP binding protein-like 5, an ATP23 peptidase, an autophagin-1, an autophagin-2, an autophagin-3, an autophagin-4, an azurocidin, a beta lactamase, a beta-secretase 1, a beta-secretase 2, a bleomycin hydrolase, a brain serine proteinase 2, a BRCC36 (BRCA2-containing complex, sub 3), a calpain, a calpain 1, a calpain 2, a calpain 3, a calpain 4, a calpain 5, a calpain 6, a calpain 7, a calpain 7-like, a calpain 8, a calpain 9, a calpain 10, a calpain 11, a calpain 12, a calpain 13, a calpain 14, a calpain 15 (Solh protein), a cysteine protease, a carboxypeptidase A1, a carboxypeptidase A2, a carboxypeptidase A3, a carboxypeptidase A4, a carboxypeptidase A5, a carboxypeptidase A6, a carboxypeptidase B, a carboxypeptidase D, a carboxypeptidase E, a carboxypeptidase M, a carboxypeptidase N, a carboxypeptidase O, a carboxypeptidase U, a carboxypeptidase X1, a carboxypeptidase X2, a carboxypeptidase Z, a carnosine dipeptidase 1, a carnosine dipeptidase 2, a caspase recruitment domain family, member 8, a caspase, a caspase-1, a caspase-2, a caspase-3, a caspase-4/11, a caspase-5, a caspase-6, a caspase-7, a caspase-8, a caspase-9, a caspase-10, a caspase-12, a caspase-14, a caspase-14-like, a casper/FLIP, a cathepsin, a cathepsin A (CTSA), a cathepsin B (CTSB), a cathepsin C (CTSC), a cathepsin D (CTSD), a cathepsin E (CTSE), a cathepsin F, a cathepsin G, a cathepsin H (CTSH), a cathepsin K (CTSK), a cathepsin L (CTSL), a cathepsin L2, a cathepsin O, a cathepsin S (CTSS), a cathepsin V (CTSV), a cathepsin W, a cathepsin Z (CTSZ), a cationic trypsin, a cezanne/OTU domain containing 7B, a cezanne-2, a CGI-58, a chymase, a chymopasin, a chymosin, a chymotrypsin B, a chymotrypsin C, a coagulation factor IXa, a coagulation factor VIIa, a coagulation factor Xa, a coagulation factor XIa, a coagulation factor XIIa, a collagenase 1, a collagenase 2, a collagenase 3, a complement protease C1r serine protease, a complement protease C1s serine protease, a complement C1r-homolog, a complement component 2, a complement component C1ra, a complement component C1sa, a complement factor B, a complement factor D, a complement factor D-like, a complement factor I, a COPS6, a corin, a CSN5 (JAB1), a cylindromatosis protein, a cytosol alanyl aminopep.-like 1, a cytosol alanyl aminopeptidase, a DDI-related protease, a DECYSIN, a Der1-like domain family, member 1, a Der1-like domain family, member 2, a Der1-like domain family, member 3, a DESC1 protease, a desert hedgehog protein, a desumoylating isopeptidase 1, a desumoylating isopeptidase 2, a dihydroorotase, a dihydropyrimidinase, a dihydropyrimidinase-related protein 1, a dihydropyrimidinase-related protein 2, a dihydropyrimidinase-related protein 3, a dihydropyrimidinase-related protein 4, a dihydropyrimidinase-related protein 5, a DINE peptidase, a dipeptidyl peptidase (DPP), a dipeptidyl peptidase (DPP1), a dipeptidyl-peptidase 4 (DPP4), a dipeptidyl-peptidase 6 (DPP6), a dipeptidyl-peptidase 8 (DPP8), a dipeptidyl-peptidase 9 (DPP9), a dipeptidyl-peptidase II, a dipeptidyl-peptidase III, a dipeptidyl-peptidase 10 (DPP10), a DJ-1, a DNA-damage inducible protein, a DNA-damage inducible protein 2, a DUB-1, a DUB-2, a DUB2a, a DUB2a-like, a DUB2a-like2, a DUB6, or a combination thereof.

89. The method of claim 87, wherein the protease is selected from the group consisting of a T cell protease, a complement protease, a fibrosis protease, and an inflammation-related protease.

90. The method of claim 78, wherein an NHS group reacts with the free amino group, thereby covalently binding the linker to the synthetic polymer.

91. The method of claim 78, wherein the synthetic polymer comprises a dendrimer, a multivalent synthetic macromolecule, a nanoparticle scaffold, a polymeric scaffold, a foldamer, a branched peptide, or a synthetic composite nanoparticle.

92. The method of claim 78, wherein the synthetic polymer further comprises a probe.

93. The method of claim 92, wherein the probe is selected from Table 1.

94. The method of claim 92, wherein the synthetic polymer comprises an IEPD dendrimer, and wherein the probe comprises a probe 9 molecule, a probe 102 molecule, or a probe 379 molecule, or a combination thereof.

95. The synthetic molecule of claim 78, wherein the synthetic polymer further comprises a plurality of probes.

96. The synthetic molecule of claim 95, wherein the plurality of probes are selected from Table 1.
